(19) United States
(12) Patent Application Publication
(10) Pub. No.: US 2002/0103185 A1
(43) Pub. Date: Aug. 1, 2002

(54) Pyrazole derivatives

(76) Inventors: Mark A. Sanner, Old Saybrook, CT (US); Chris J. Helal, Mystic, CT (US); Christopher B. Cooper, Lawrenceville, NJ (US); Travis T. Wager, New London, CT (US)

Correspondence Address: PFIZER INC, 150 EAST 42ND STREET, 5TH FLOOR - STOP 49, NEW YORK, NY, 10017-5612, US

(21) Appl. No.: 09/941,001

(22) Filed: Aug. 28, 2001

Related U.S. Application Data
(60) Provisional application No. 60/229,415, filed on Aug. 31, 2000.
(60) Provisional application No. 60/232,032, filed on Sep. 12, 2000.

(51) Int. Cl. A61K 31/55; A61K 31/454; C 07D 4 3/02; A61K 31/415

(52) U.S. Cl. 514/217.09; 514/407; 514/326; 540/603; 548/364.1; 548/367.4; 546/211

(57) ABSTRACT

The invention provides compounds of formula 1

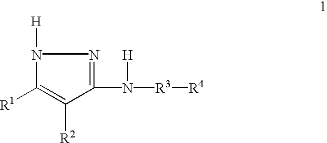

wherein R¹, R², R³, and R⁴ are as defined, and their pharmaceutically acceptable salts. Compounds of formula 1 are indicated to have activity inhibiting cdk5, cdk2, and GSK-3. Pharmaceutical compositions and methods comprising compounds of formula 1 for treating and preventing diseases and conditions comprising abnormal cell growth, such as cancer, and neurodegenerative diseases and conditions and those affected by dopamine neurotransmission. Also described are pharmaceutical compositions and methods comprising compounds of formula 1 for treating male fertility and sperm motility; diabetes mellitus; impaired glucose tolerance; metabolic syndrome or syndrome X; polycystic ovary syndrome; adipogenesis and obesity; myogenesis and frailty, for example age-related decline in physical performance; acute sarcopenia, for example muscle atrophy and/or cachexia associated with burns, bed rest, limb immobilization, or major thoracic, abdominal, and/or orthopedic surgery; sepsis; hair loss, hair thinning, and balding; and immunodeficiency.

This application claims priority under 35 U.S.C. 119(e) of U.S. Provisional Application No. 60/229,415, filed Aug. 31, 2000, and of U.S. Provisional Application No.60/232,032, filed Sep. 12, 2000.

### FIELD OF THE INVENTION

The subject invention relates to pyrazole derivatives, pharmaceutical compositions comprising such derivatives and methods of using such derivatives to treat abnormal cell growth and certain diseases and conditions of the central nervous system. The compounds of the present invention act as inhibitors of cyclin-dependent protein kinase enzymes cdk5 (cyclin-dependent protein kinase 5) and cdk2 (cyclin-dependent protein kinase 2). The compounds of the present invention also are inhibitors of the enzyme GSK-3 (glygocen synthase kinase-3) enzyme.

### BACKGROUND OF THE INVENTION

The serine/threonine kinase cdk5 along with its cofactor p25 (or the longer cofactor, p35) has been linked to neurodegenerative disorders, and inhibitors of cdk5/p25 (or cdk5/p35) are therefore useful for the treatment of neurodegenerative disorders such as Alzheimer's disease, Parkinson's disease, stroke, or Huntington's disease. Treatment of such neurodegenerative disorders using cdk5 inhibitors is supported by the finding that cdk5 is involved in the phosphorylation of tau protein (J. Biochem, 117, 741-749 (1995)). cdk5 also phosphorylates Dopamine and Cyclic AMP-Regulated Phosphorprotein (DARPP-32) at threonine 75 and is thus indicated in having a role in dopaminergic neurotransmission (Nature, 402, 669-671 (1999)).

The serine/threonine kinase cdk2 is essential for normal cell cycling and plays a critical role in disorders arising from abnormal cell cycling, a common characteristic of many oncological disorders. Inhibitors of cdk2 are therefore useful for the treatment of various types of cancer and other diseases or conditions related to abnormal cell growth (Meijer, et al., Properties and Potential-applications of Chemical Inhibitors of Cyclin-dependent Kinsases, Pharmacology & therapeutics, 82 (2-3), 279-284 (1999); Sausville, et al., Cyclin-dependent Kinases: Initial Approaches to Exploit a Novel Therapeutic Target, Pharmacology & therapeutics 82 (2-3) 285-292 (1999)).

GSK-3 is a serine/threonine protein kinase. It is one of several protein kinases which phosphorylate glycogen synthase (Embi, et al., Eur. J. Biochem. 107:519-527 (1980); Hemmings, et al., Eur. J. Biochem. 119:443-451 (1982)). GSK-3 exists in two isoforms, a and β, in vertebrates, reported as having a monomeric structure of 49 kD and 47 kD respectively. Both isoforms phosphorylate muscle glycogen synthase (Cross, et al., Biochemical Journal 303: 21-26 (1994)). The amino acid identity among GSK-3 species homologs has been indicated to be in excess of 98% within the catalytic domain (Plyte, et al., Biochim. Biophys. Acta 1114:147-162) (1992)). Due to a remarkably high degree of conservation across the phylogenetic spectrum, a fundamental role of GSK-3 in cellular processes is suggested.

GSK-3 has been implicated in numerous different disease states and conditions. For example, Chen, et al, Diabetes 43:1234-1241 (1994) have suggested that an increase in GSK-3 activity can be important in Type 2 diabetes. Increased GSK-3 expression in diabetic muscle is also though to contribute to the impaired glycogen synthase activity and skeletal muscle insulin resistance present in Type 2 diabetes (Nikoulina, et al., Diabetes 49: 263-271 (2000)). Also, a higher activity of a type 1 protein phosphatase measured in immotile sperm was attributed to higher GSK-3 activity and was indicated as responsible for holding the sperm motility in check (Vijayaraghavan, et al. Biology of Reproduction 54: 709-718 (1996)). Vijayaraghavan et al. indicate that such results suggest a biochemical basis for the development and regulation of sperm motility and a possible physiological role for a protein phosphatase 1/inhibitor 2/GSK-3 system. GSK-3 activity has also been associated with Alzheimer's disease and mood disorders such as bipolar disorder (WO 97/41854). Among other conditions, GSK-3 has furthermore been implicated in hair loss, schizophrenia, and neurodegeneration, including both chronic neurodegenerative diseases (such as Alzheimer's, supra) and neurotrauma, for example stroke, traumatic brain injury, and spinal cord trauma.

### SUMMARY OF THE INVENTION

This invention provides compounds of the formula

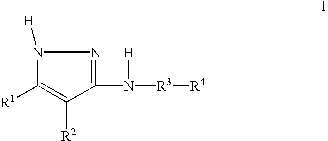

wherein R¹ is a straight chain or branched (C₁-C₈)alkyl, a straight chain or branched (C₂-C₈)alkenyl, a straight chain or branched (C₂-C₈)alkynyl, (C₃-C₈)cycloalkyl, (C₄-C₈)cycloalkenyl, 3-8 membered) heterocycloalkyl, (C₅-C₁₁)bicycloalkyl, (C₇-C₁₁)bicycloalkenyl, or (5-11 membered) heterobicycloalkyl; and wherein R¹ is optionally substituted with from one to six substituents R⁵ independently selected from F, Cl, Br, I, nitro, cyano, —CF₃, —NR⁷R⁸, —NR⁷C(═O)R⁸, —NR⁷C(═O)OR⁸, —NR⁷C(═O)NR⁸R⁹, —NR⁷S(═O)₂R⁸, —NR⁷S(═O)₂NR⁸R⁹, —OR⁷, —OC(═)R⁷, OC(═O)OR⁷, —C(═O)OR⁷, —C(═O)R⁷, —C(═O)NR⁷R⁸, —OC(═O)NR⁷R⁸, —OC(═O)SR⁷, —SR⁷, —S(═O)R⁷, —S(═O)₂R⁷, —S(═O)₂NR⁷R⁸, and R⁷;

R² is H, F, —CH₃, —CN, or —C(═O)OR⁷;

R³ is —C(═O)NR⁹—, —C(═O)O—, —C(═O)(CR¹⁰R¹¹)ₙ—, or —(CR¹⁰R¹¹)ₙ—;

R⁴ is a straight chain or a branched (C₁-C₈)alkyl, a straight chain or a branched (C₂-C₈)alkenyl, a straight chain or branched (C₂-C₈ alkynyl), (C₃-C₈)cycloalkyl, (C₄-C₈)cycloalkenyl, (3-8 membered) heterocycloalkyl, (C₅-C₁₁)bicycloalkyl, (C₇-C₁₁)bicycloalkenyl, (5-11 membered) heterobicycloalkyl, (C₆-C₁₄)aryl, or (5-14 membered) heteroaryl; and wherein R⁴ is optionally substituted with from one to three substitutents R⁶ independently selected from F, Cl, Br, I, nitro, cyano, —CF₃, —NR⁷R⁸, —NR⁷C(═O)R⁸, —NR⁷C(═O)OR⁸, —NR⁷C(═O)NR⁸R⁹, —NR⁷S(═O)₂R⁸, —NR⁷S(═O)₂NR⁸R⁹, —OR⁷, —OC(═O)R⁷, —OC(═O)OR⁷, —C(═O)OR⁷, —C(═O)R⁷, —C(═O)NR⁷R⁸, —OC(═O)NR⁷R⁸, —OC(═O)SR⁷, —SR⁷, —S(═O)R⁷, —S(═O)₂R⁷, —S(═O)₂NR⁷R⁸, or R⁷;

each R⁷, R⁸, and R⁹ is independently selected from H, straight chain or branched (C₁-C₈)alkyl, straight chain or branched (C₂-C₈)alkenyl, straight chain or branched (C₂-C₈ alkynyl), (C₃-C₈)cycloalkyl, (C₄-C₈)cycloalkenyl, (3-8 membered) heterocycloalkyl, (C₅-C₁₁)bicycloalkyl, (C₇-C₁₁)bicycloalkenyl, (5-11 membered) heterobicycloalkyl, (C₆-C₁₄)aryl, and (5-14 membered) heteroaryl, wherein R⁷, R⁸, and R⁹ are each independently optionally substituted with from one to six substituents independently selected from F, Cl, Br, I, NO₂, —CN, —CF₃, —NR¹⁰R¹¹, —NR¹⁰C(═O)R¹¹, —NR¹⁰C(═O)OR¹¹, —NR¹⁰C(═O)NR¹¹R¹², —NR¹⁰S(═O)₂R¹¹, —NR¹⁰S(═O)₂NR¹¹R¹², —OR¹⁰, —OC(═O)R¹⁰, —OC(═O)OR¹⁰, —OC(═O)NR¹⁰R¹¹, —OC(═O)SR¹⁰, —SR¹⁰, —S(═O)R¹⁰, —S(═O)₂R¹⁰, —S(═O)₂NR¹⁰R¹¹, —C(═O)R¹⁰, —C(═O)OR¹⁰, —C(═O)NR¹⁰R¹¹, and R¹⁰;

or, when R⁷ and R⁸ are as in NR⁷R⁸, they may instead optionally be connected to form with the nitrogen of NR⁷R⁸ to which they are attached a heterocycloalkyl moiety of from three to seven ring members, said heterocycloalkyl moiety optionally comprising one or two further heteroatoms independently selected from N, O, and S;

each R¹⁰, R¹¹, and R¹² is independently selected from H, straight chain or branched (C₁-C₈)alkyl, straight chain or branched (C₂-C₈)alkenyl, straight chain or branched (C₂-C₈ alkynyl), (C₃-C₈)cycloalkyl, (C₄-C₈)cycloalkenyl, (3-8 membered) heterocycloalkyl, (C₅-C₁₁)bicycloalkyl, (C₇-C₁₁)bicycloalkenyl, (5-11 membered) heterobicycloalkyl, (C₆-C₁₄)aryl, and (5-14 membered) heteroaryl, wherein R¹⁰, R¹¹, and R¹² are each independently optionally substituted with from one to six substituents independently selected from F, Cl, Br, I, NO₂, —CN, —CF₃, —NR¹³R¹⁴, —NR¹³C(═O)R¹⁴, —NR¹³C(═O)OR¹⁴, —NR¹³C(═O)NR¹⁴R¹⁵, —NR¹³S(═O)₂R¹⁴, —NR¹³S(═O)₂NR¹⁴R¹⁵, —OR¹³, —OC(═O)R¹³, —OC(═O)OR¹³, —OC(═O)NR¹³R¹⁴, —OC(═O)SR¹³, —SR¹³, —S(═O)R¹³, —S(═O)₂R¹³, —S(═O)₂NR¹³R¹⁴, —C(═O)R¹³, —C(═O)OR¹³, —C(═O)NR¹³R¹⁴, and R¹³;

each R¹³, R¹⁴, and R¹⁵ is independently selected from H, straight chain or branched (C₁-C₈)alkyl, straight chain or branched (C₂-C₈)alkenyl, straight chain or branched (C₂-C₈ alkynyl), (C₃-C₈)cycloalkyl, (C₄-C₈)cycloalkenyl, (3-8 membered) heterocycloalkyl, (C₅-C₁₁)bicycloalkyl, (C₇-C₁₁)bicycloalkenyl, (5-11 membered) heterobicycloalkyl, (C₆-C₁₄)aryl, and (5-14 membered) heteroaryl, wherein R¹³, R¹⁴, and R¹⁵ are each independently optionally substituted with from one to six substituents independently selected from F, Cl, Br, I, NO₂, —CN, —CF₃, —NR¹⁶R¹⁷, —NR¹⁶C(═O)R¹⁷, —NR¹⁶C(═O)OR¹⁷, —NR¹⁶C(═O)NR¹⁷R¹⁸, —NR¹⁶S(═O)₂R¹⁷, —NR¹⁶S(═O)₂NR¹⁷R¹⁸ —OR¹⁶, —OC(═O)R¹⁶, —OC(═O)OR¹⁶, —OC(═O)NR¹⁶R¹⁷, —OC(═O)SR¹⁶, —SR¹⁶, —S(═O)R¹⁶, —S(═O)₂R¹⁶, —S(═O)₂NR¹⁶R¹⁷, —C(═O)R¹⁶, —C(═O)OR¹⁶, —C(═O)NR¹⁶R¹⁷, and R¹⁶

each R¹⁶, R¹⁷, and R¹⁸ is independently selected from H, straight chain or branched (C₁-C₈)alkyl, straight chain or branched (C₂-C₈)alkenyl, straight chain or branched (C₂-C₈ alkynyl), (C₃-C₈)cycloalkyl, (C₄-C₈)cycloalkenyl, (3-8 membered) heterocycloalkyl, (C₅-C₁₁)bicycloalkyl, (C₇-C₁₁)bicycloalkenyl, (5-11 membered) heterobicycloalkyl, (C6-C₄)aryl, and (5-14 membered) heteroaryl;

n is 0, 1, 2, or 3;

wherein R¹⁰ and R¹¹ in —C(═O)(CR¹⁰R¹¹)ₙ— and —(CR¹⁰R¹¹)ₙ— are for each iteration of n defined independently as recited above;

and pharmaceutically acceptable salts thereof.

Amino-substituted pyrazoles can exist as mixtures of tautomeric isomers in equilibrium with one another. The present invention includes all such tautomers of compounds of formula 1, and references herein to compounds of formula 1, unless otherwise indicated, encompass also the tautomers of compounds of formula 1.

Compounds of formula 1 of the invention are inhibitors of serine/threonine kinases, especially cyclin-dependent kinases such as cdk5 and cdk2, and are useful for the treatment of neurodegenerative disorders and other CNS disorders, and of abnormal cell growth, including cancer. The compounds of formula 1 are particularly useful in inhibiting cdk5. The compounds of formula 1 are also useful as inhibitors of GSK-3.

The term “alkyl”, as used herein, unless otherwise indicated, includes saturated monovalent hydrocarbon radicals having straight or branched moieties. Examples of alkyl groups include, but are not limited to, methyl, ethyl, propyl, isopropyl, and t-butyl.

The term “alkenyl”, as used herein, unless otherwise indicated, includes alkyl moieties having at least one carbon-carbon double bond wherein alkyl is as defined above. Examples of alkenyl include, but are not limited to, ethenyl and propenyl.

The term “alkynyl”, as used herein, unless otherwise indicated, includes alkyl moieties having at least one carbon-carbon triple bond wherein alkyl is as defined above. Examples of alkynyl groups include, but are not limited to, ethynyl and 2-propynyl.

The term “cycloalkyl”, as used herein, unless otherwise indicated, includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl. “Bicycloalkyl” groups are non-aromatic saturated carbocyclic groups consisting of two rings, wherein said rings share one or two carbon atoms. For purposes of the present invention, and unless otherwise indicated, bicycloalkyl groups include spiro groups and fused ring groups. Examples of bicycloalkyl groups include, but are not limited to, bicyclo-[3.1.0]-hexyl, norbornyl, spiro[4.5]decyl, spiro[4.4]nonyl, spiro[4.3]octyl, and spiro[4.2]heptyl. “Cycloalkenyl” and “bicycloalkenyl” refer to non-aromatic carbocyclic cycloalkyl and bicycloalkyl moieties as defined above, except comprising one or more carbon-carbon double bonds connecting carbon ring members (an “endocyclic” double bond) and/or one or more carbon-carbon double bonds connecting a carbon ring member and an adjacent non-ring carbon (an “exocyclic” double bond). Examples of cycloalkenyl groups include, but are not limited to, cyclopentenyl and cyclobutenyl, and a non-limiting example of a bicycloalkenyl group is norbornenyl. Cycloalkyl, cycloalkenyl, bicycloalkyl, and bicycloalkenyl groups also include groups that are substituted with one or more oxo moieties. Examples of such groups with oxo moieties are oxocyclopentyl, oxocyclobutyl, oxocyclopentenyl, and norcamphoryl.

The term “aryl”, as used herein, unless otherwise indicated, includes an organic radical derived from an aromatic hydrocarbon by removal of one hydrogen, such as phenyl, naphthyl, indenyl, and fluorenyl.

The terms “heterocyclic”, “heterocycloalkyl”, and like terms, as used herein, refer to non-aromatic cyclic groups containing one or more heteroatoms, prefereably from one to four heteroatoms, each selected from O, S and N. “Heterobicycloalkyl” groups are non-aromatic two-ringed cyclic groups, wherein said rings share one or two atoms, and wherein at least one of the rings contains a heteroatom (O, S, or N). Heterobicycloalkyl groups for purposes of the present invention, and unless otherwise indicated, include spiro groups and fused ring groups. In one embodiment, each ring in the heterobicycloalkyl contains up to four heteroatoms (i.e. from zero to four heteroatoms, provided that at least one ring contains at least one heteroatom). The heterocyclic groups of this invention can also include ring systems substituted with one or more oxo moieties. Examples of non-aromatic heterocyclic groups are aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl.

“Heteroaryl”, as used herein, refers to aromatic groups containing one or more heteroatoms (O, S, or N), preferably from one to four heteroatoms. A multicyclic group containing one or more heteroatoms wherein at least one ring of the group is aromatic is a “heteroaryl” group. The heteroaryl groups of this invention can also include ring systems substituted with one or more oxo moieties. Examples of heteroaryl groups are pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

The foregoing groups, as derived from the compounds listed above, may be C-attached or N-attached where such is possible. For instance, a group derived from pyrrole may be pyrrol-1-yl (N-attached) or pyrrol-3-yl (C-attached). The terms referring to the groups also encompass all possible tautomers.

In one embodiment, this invention provides compounds of formula 1, wherein R³ is —C(═O)NR⁹— or —C(═O)(CR¹⁰R¹¹)ₙ—. In another embodiment, R¹⁰ and R¹¹ of —C(═O)(CR¹⁰OR¹¹)ₙ— are at each iteration of n both hydrogen. In another embodiment, R⁹ of —C(═O)NR⁹— is hydrogen. In another embodiment, R³ is —C(═O)NR⁹— or —C(═O)(CR¹⁰R¹¹)ₙ— and R² is hydrogen. In another embodiment, R³ is —(CR¹⁰R¹¹)ₙ—, and n is zero. In a preferred embodiment R³ is —(CR¹⁰R¹¹)ₙ—, n is zero, and R⁴ is (C₆-C₁₄)aryl or (5-14 membered) heteroaryl, each optionally substituted as recited above.

In another embodiment of the invention, a compound of formula 1 is provided wherein R¹ is optionally substituted (C₃-C₈)cycloalkyl or optionally substituted (C₅-C₁₁) bicycloalkyl. Preferred embodiments are wherein R¹ is cyclopropyl, cyclobutyl, cyclopentyl, cyclohexyl, or norbornyl, each optionally substituted as recited above (i.e. optionally with from one to six substituents R⁵ independently selected from F, Cl, Br, I, nitro, cyano, —CF₃, —NR⁷R⁸, —NR⁷C(═O)R⁸, —NR⁷C(═O)OR⁸, —NR⁷C(═O)NR⁸R⁹, —NR⁷S(═O)₂R⁸, —NR⁷S(═O)₂NR⁸R⁹, —OR⁷, —OC(═O)R⁷, —OC(═O)OR⁷, —C(═O)OR⁷, —C(═O)R⁷, —C(═O)N R⁷R⁸, —OC(═O)NR⁷R⁸, —OC(═O)SR⁷, —SR⁷, —S(═O)R⁷, —S(═O)₂R⁷, —S(═O)₂NR⁷R⁸, and R⁷). In a more preferred embodiment R¹ is (C₃-C₈)cycloalkyl or optionally substituted (C₅-C₁₁) bicycloalkyl, for example cyclopropyl, cyclobutyl, cyclopentyl, cyclohexyl, or norbornyl, and is optionally substituted with from one to three substituents independently selected from F, Cl, Br, I, nitro, cyano, —CF₃, —NR⁷R⁸, —NR⁷C(═O)R⁸, —OR⁷, —C(═O)OR⁷, —C(═O)R⁷, and R⁷. More preferably, R¹ is (C₃-C₈)cycloalkyl or optionally substituted (C₅-C₁₁) bicycloalkyl, for example cyclopropyl, cyclobutyl, cyclopentyl, cyclohexyl, or norbornyl, and R¹ is substituted with —NR⁷C(═O)R⁸, (C₆-C₁₄)aryl, (3-8 membered) heterocycloalkyl, or (5-14 membered) heteroaryl, and wherein said aryl, heterocycloalkyl, and heteroaryl are each optionally substituted with from one to six substituents independently selected from F, Cl, Br, I, NO₂, —CN, —CF₃, —NR¹⁰R¹¹, —NR¹⁰C(═O)R¹¹, —NR¹⁰C(═O)OR¹¹, —NR¹⁰C(═O)NR¹¹R¹², —NR¹⁰S(═O)₂R¹¹, —NR¹⁰S(═O)₂NR¹¹R¹², —OR¹⁰, —OC(═O)R¹⁰, —OC(═O)OR¹⁰, —OC(═O)NR¹⁰R¹¹, —OC(═O)SR¹⁰, —SR¹⁰, —S(═O)R¹⁰, —S(═O)₂R¹⁰, —S(═O)₂NR¹⁰R¹¹, —C(═O)R¹⁰, —C(═O)OR¹⁰, —C(═O)NR¹⁰OR¹¹, and R¹⁰. In another embodiment of the invention, R¹ is bicyclo-[3.1.0]-hexyl and is optionally substituted as recited above (i.e. optionally substituted with from one to six substituents R⁵ independently selected from F, Cl, Br, I, nitro, cyano, —CF₃, —NR⁷R⁸, —NR⁷C(═O)R⁸, —NR⁷C(═O)OR⁸, —NR⁷C(═O)NR⁸R⁹, —NR⁷S(═O)₂R⁸, —NR⁷S(═O)₂NR⁸R⁹, —OR⁷, —OC(═O)R⁷, —OC(═O)OR⁷, —C(═O)OR⁷, —C(═O)R⁷, —C(═O)NR⁷R⁸, —OC(═O)NR⁷R⁸, —OC(═O)SR⁷, —SR⁷, —S(═O)R⁷, —S(═O)₂R⁷, —S(═O)₂NR⁷R⁸, and R⁷.

In another embodiment of the invention, a compound of formula 1 is provided wherein R¹ is optionally substituted straight chain or branched (C₁-C₈)alkyl or optionally substituted straight chain or branched (C₂-C₈)alkenyl.

In another embodiment of the invention, compounds of formula 1 are provided, but wherein R² is hydrogen. In a further embodiment, R² is hydrogen, and R¹ is as subdefined in the preceding paragraphs.

In another embodiment, this invention provides a compound of formula 1 wherein R⁴ is (C₆-C₁₄)aryl or (5-14 membered) heteroaryl, each optionally substituted. In a preferred embodiment, R⁴ is optionally substituted phenyl or optionally substituted pyridyl. In another preferred embodiment, R⁴ is naphthyl, quinolyl, or isoquinolyl, each optionally substituted. In another embodiment, R⁴ is napthyl, quinolyl, or isoquinolyl, and is unsubstituted. In another embodiment, R⁴ is pyrimidinyl, pyrazinyl, or pyridazyl, and in each case R⁴ is optionally substituted. In a further embodiment, R⁴ is pyrimidinyl, pyrazinyl, or pyridazyl, and R⁴ is unsubstituted.

In another embodiment, compounds of formula 1 are provided, wherein R² is specifically hydrogen, and R⁴ is as subdefined in the preceding paragraph.

Examples of preferred compounds of formula 1 are:

(5-cyclobutyl-2H-pyrazol-3-yl)-(3-trifluoromethoxy-phenyl)-amine;

N-(5-cyclobutyl-2H-pyrazol-3-yl)-N′,N′-dimethyl-pyridine-2,6-diamine;

(5-ethyl-2H-pyrazol-3-yl)-(6-methoxy-pyridin-2-yl)-amine;

(5-cyclobutyl-2H-pyrazol-3-yl)-(6-methoxy-pyridin-2-yl)-amine

(5-cyclobutyl-2H-pyrazol-3-yl)-naphthalen-2-yl-amine;

(5-cyclobutyl-2H-pyrazol-3-yl)-naphthalen-1-yl-amine;

N-(5-cyclobutyl-2H-pyrazol-3-yl)-N′,N′-dimethyl-naphthalene-1,4-diamine;

N-(5-cyclobutyl-2H-pyrazol-3-yl)-N′, N′-dimethyl-pyridine-2,6-diamine;

(5-cyclobutyl-2H-pyrazol-3-yl)-(6-trifluoromethyl-pyridin-2-yl)-amine;

(3-benzyloxy-phenyl)-(5-cyclobutyl-2H-pyrazol-3-yl)-amine;

(5-cyclobutyl-2H-pyrazol-3-yl)-(3-trifluoromethyl-phenyl)-amine;

N-(5-cyclobutyl-2H-pyrazol-3-yl)-N′,N′-dimethyl-benzene-1,3-diamine;

(5-cyclobutyl-2H-pyrazol-3-yl)-(3-methoxy-phenyl)-amine;

(5-cyclobutyl-2H-pyrazol-3-yl)-(4-nitro-phenyl)-amine;

(4-chloro-benzyl)-(5-cyclobutyl-2H-pyrazol-3-yl)-amine;

(3-bromo-phenyl)-(5-cyclobutyl-2H-pyrazol-3-yl)-amine;

(5-cyclobutyl-2H-pyrazol-3-yl)-quinolin-2-yl-amine;

[5-(1,4-dioxa-spiro[4.4]non-7-yl)-1H-pyrazol-3-yl]-(3-trifluoromethyl-phenyl)-amine;

(6-chloro-pyridin-2-yl)-(5-cyclobutyl-2H-pyrazol-3-yl)-amine;

3-[5-(3-trifluoromethyl-phenylamino)-2H-pyrazol-3-yl]-cyclopentanone;

(5-cyclobutyl-2H-pyrazol-3-yl)-(6-methoxy-4-methyl-quinolin-2-yl)-amine;

(5-cyclobutyl-2H-pyrazol-3-yl)-(3-trifluoromethoxy-phenyl)-amine;

(2-chloro-4-nitro-phenyl)-(5-cyclobutyl-2H-pyrazol-3-yl)-amine;

3-trans-[5-(3-trifluoromethyl-phenylamino)-2H-pyrazol-3-yl]-cyclopentanol;

(3,5-bis-trifluoromethyl-phenyl)-(5-cyclobutyl-2H-pyrazol-3-yl)-amine;

[5-(3-cis-benzylamino-cyclopentyl)-1H-pyrazol-3-yl]-(3-trifluoromethyl-phenyl)-amine;

{5-[3-cis-(4-methoxy-benzylamino)-cyclopentyl]-1H-pyrazol-3-yl}-(3-trifluoromethyl-phenyl)-amine;

4-(5-cyclobutyl-2H-pyrazol-3-ylamino)-benzonitrile;

(5-cyclobutyl-2H-pyrazol-3-yl)-(3-fluoro-phenyl)-amine;

(5-cyclobutyl-2H-pyrazol-3-yl)-(3,5-dichloro-phenyl)-amine;

(2-bromo-phenyl)-(5-cyclobutyl-2H-pyrazol-3-yl)-amine;

N-{cis-3-[5-(3-trifluoromethyl-phenylamino)-2H-pyrazol-3-yl]-cyclopentyl}-acetamide;

pyridin-2-yl-{3-trans-[5-(3-trifluoromethyl-phenylamino)-2H-pyrazol-3-yl]-cyclopentyl}-amine;

(5-cyclobutyl-1H-pyrazol-3-yl)-(4-methoxy-phenyl)-amine;

pyridine-2-carboxylic acid {3-[5-(3-trifluoromethyl-phenylamino)-2H-pyrazol-3-yl ]-cyclopentyl}-amide;

3-trifluoromethyl-N-{3-[5-(3-trifluoromethyl-phenylamino)-2H-pyrazol-3-yl ]-cyclopentyl}-benzamide;

cyclobutanecarboxylic acid {3-[5-(3-trifluoromethyl-phenylamino)-2H-pyrazol-3-yl ]-cyclopentyl}-amide;

2,2-dimethyl-N-{3-[5-(3-trifluoromethyl-phenylamino)-2H-pyrazol-3-yl]-cyclopentyl }-propionamide;

4-fluoro-N-{3-[5-(3-trifluoromethyl-phenylamino)-2H-pyrazol-3-yl]-cyclopentyl}-benzamide;

2,2,2-trifluoro-N-{3-[5-(3-trifluoromethyl-phenylamino)-2H-pyrazol-3-yl]-cyclopentyl }-acetamide;

cyclopropanecarboxylic acid {3-[5-(3-trifluoromethyl-phenylamino)-2H-pyrazol-3-yl ]-cyclopentyl}-amide;

N-{3-[5-(3-trifluoromethyl-phenylamino)-2H-pyrazol-3-yl]-cyclopentyl}-propionamide;

cyclohexanecarboxylic acid {3-[5-(3-trifluoromethyl-phenylamino)-2H-pyrazol-3-yl ]-cyclopentyl}-amide;

N-[5-(3-acetylamino-cyclopentyl)-2H-pyrazol-3-yl]-2-naphthalen-1-yl-acetamide;

cyclopropanecarboxylic acid {3-[5-(2-naphthalen-1-yl-acetylamino)-1H-pyrazol-3-yl ]-cyclopentyl}-amide;

2-naphthalen-1-yl-N-{5-[3-(2,2,2-trifluoro-acetylamino)-cyclopentyl]-2H-pyrazol-3-yl }-acetamide;

N-{3-[5-(2-naphthalen-1-yl-acetylamino)-1H-pyrazol-3-yl]-cyclopentyl}-benzamide;

N-(5-hydroxymethyl-1H-pyrazol-3-yl)-2-naphthalen-1-yl-acetamide;

2-naphthalen-1-yl-N-[5-(thiazol-2-ylaminomethyl)-1H-pyrazol-3-yl]-acetamide;

N-[5-((1S)-hydroxy-ethyl)-2H-pyrazol-3-yl]-2-naphthalen-1-yl-acetamide;

N-{5-[(1S)-(benzooxazol-2-yloxy)-ethyl]-1H-pyrazol-3-yl}-2-naphthalen-1-yl-acetamide;

N-{5-[(1S)-(benzothiazol-2-yloxy)-ethyl]-1H-pyrazol-3-yl}-2-naphthalen-1-yl-acetamide;

N-[5-(3-hydroxy-1-methyl-propyl)-1H-pyrazol-3-yl]-2-naphthalen-1-yl-acetamide;

N-[5-(benzothiazol-2-yloxymethyl)-1H-pyrazol-3-yl]-2-naphthalen-1-yl-acetamide;

N-{5-[3-(benzothiazol-2-yloxy)-1-methyl-propyl]-1H-pyrazol-3-yl}-2-naphthalen-1-yl-acetamide;

N-[5-(2-hydroxy-(1S)-methyl-ethyl)-2H-pyrazol-3-yl]-2-naphthalen-1-yl-acetamide;

N-{5-[(1R)-(benzothiazol-2-yloxy)-ethyl]-1H-pyrazol-3-yl}-2-naphthalen-1-yl-acetamide;

N-[5-(3-acetylamino-1-methyl-propyl)-1H-pyrazol-3-yl]-2-naphthalen-1-yl-acetamide;

3-methoxy-N-{cis-3-[5-(2-naphthalen-1-yl-acetylamino)-2H-pyrazol-3-yl]-cyclobutyl}-benzamide;

N-[5-(cis-3-acetylamino-cyclobutyl)-1H-pyrazol-3-yl]-2-naphthalen-1-yl-acetamide;

N-{cis-3-[5-(2-naphthalen-1-yl-acetylamino)-2H-pyrazol-3-yl]-cyclobutyl}-benzamide;

2-cyclopropyl-N-{cis-3-[5-(2-naphthalen-1-yl-acetylamino)-2H-pyrazol-3-yl]-cyclobutyl}-acetamide;

6-chloro-pyridine-2-carboxylic acid {cis-3-[5-(2-naphthalen-1-yl-acetylamino)-2H-pyrazol-3-yl]-cyclobutyl}-amide;

quinoline-2-carboxylic acid {cis-3-[5-(2-naphthalen-1-yl-acetylamino)-2H-pyrazol-3-yl]-cyclobutyl}-amide;

pyrazine-2-carboxylic acid {cis-3-[5-(2-naphthalen-1-yl-acetylamino)-2H-pyrazol-3-yl]-cyclobutyl}-amide;

4-methoxy-N-{cis-3-[5-(2-naphthalen-1-yl-acetylamino)-2H-pyrazol-3-yl]-cyclobutyl}-benzamide;

N-{cis-3-[5-(2-naphthalen-1-yl-acetylamino)-2H-pyrazol-3-yl]-cyclobutyl}-3-nitro-benzamide;

N-{cis-3-[5-(2-naphthalen-1-yl-acetylamino)-2H-pyrazol-3-yl]-cyclobutyl}-3-trifluoromethyl-benzamide;

N-{cis-3-[5-(2-naphthalen-1-yl-acetylamino)-2H-pyrazol-3-yl]-cyclobutyl}-isobutyramide;

2-phenyl-cyclopropanecarboxylic acid {cis-3-[5-(2-naphthalen-1-yl-acetylamino)-2H-pyrazol-3-yl]-cyclobutyl}-amide;

N-{5-[cis-3-(benzooxazol-2-yloxy)-cyclobutyl]-1H-pyrazol-3-yl}-2-naphthalen-1-yl-acetamide;

4-dimethylamino-N-{cis-3-[5-(2-naphthalen-1-yl-acetylamino)-2H-pyrazol-3-yl]-cyclobutyl}-benzamide;

3,5-dimethoxy-N-{cis-3-[5-(2-naphthalen-1-yl-acetylamino)-2H-pyrazol-3-yl]-cyclobutyl}-benzamide;

2-naphthalen-1-yl-N-[5-(cis-3-phenyl-cyclobutyl)-2H-pyrazol-3-yl]-acetamide;

N-{5-[cis-3-(3-methoxy-phenyl)-cyclobutyl]-2H-pyrazol-3-yl}-2-naphthalen-1-yl-acetamide;

N-{5-[cis-3-(2-methoxy-phenyl)-cyclobutyl]-2H-pyrazol-3-yl}-2-naphthalen-1-yl-acetamide;

N-{5-[cis-3-(4-methoxy-phenyl)-cyclobutyl]-2H-pyrazol-3-yl}-2-naphthalen-1-yl-acetamide;

2-naphthalen-1-yl-N-[5-(cis-3-p-tolyl-cyclobutyl)-2H-pyrazol-3-yl]-acetamide;

N-{5-[cis-3-(4-chloro-phenyl)-cyclobutyl]-2H-pyrazol-3-yl-}2-naphthalen-1-yl-acetamide;

2-(4-methoxy-phenyl)-N-{5-[cis-3-(2-methoxy-phenyl)-cyclobutyl]-2H-pyrazol-3-yl}-acetamide;

N-{5-[cis-3-(2-methoxy-phenyl)-cyclobutyl]-2H-pyrazol-3-yl}-2-quinolin-6-yl-acetamide;

N-{5-[cis-3-(2-methoxy-phenyl)-cyclobutyl]-2H-pyrazol-3-yl}-2-phenyl-acetamide;

N-{5-[cis-3-(2-methoxy-phenyl)-cyclobutyl]-2H-pyrazol-3-yl}-2-pyridin-3-yl-acetamide;

N-{5-[cis-3-(4-methoxy-phenyl)-cyclobutyl]-1H-pyrazol-3-yl}-2-quinolin-6-yl-acetamide;

2-quinolin-6-yl-N-[5-(cis-3-p-tolyl-cyclobutyl)-1H-pyrazol-3-yl]-acetamide;

N-{5-[cis-3-(4-fluoro-phenyl)-cyclobutyl]-1H-pyrazol-3-yl}-2-quinolin-6-yl-acetamide;

N-{5-[cis-3-(4-chloro-phenyl)-cyclobutyl]-1H-pyrazol-3-yl}-2-quinolin-6-yl-acetamide;

2-quinolin-6-yl-N-[5-(cis-3-m-tolyl-cyclobutyl)-1H-pyrazol-3-yl]-acetamide;

4-dimethylamino-N-{cis-3-[5-(2-naphthalen-1-yl-acetylamino)-2H-pyrazol-3-yl]-cyclobutyl}-benzamide;

2-naphthalen-1-yl-N-{5-[cis-3-(pyridin-2-yloxy)-cyclobutyl]-1H-pyrazol-3-yl}-acetamide;

6-methyl-pyridine-2-carboxylic acid {cis-3-[5-(2-naphthalen-1-yl-acetylamino)-2H-pyrazol-3-yl]-cyclobutyl}-amide;

2-phenyl-cyclopropanecarboxylic acid methyl-{cis-3-[5-(2-naphthalen-1-yl-acetylamino)-2H-pyrazol-3-yl]-cyclobutyl}-amide;

N-{5-[cis-3-(3-methyl-pyrazin-2-yloxy)-cyclobutyl]-1H-pyrazol-3-yl}-2-naphthalen-1-yl-acetamide;

{5-[cis-3-(2-methoxy-phenyl)-cyclobutyl]-1H-pyrazol-3-yl}-(6-methoxy-pyridin-2-yl)-amine;

N-{5-[cis-3-(3,6-dimethyl-pyrazin-2-yloxy)-cyclobutyl]-1H-pyrazol-3-yl}-2-naphthalen-1-yl-acetamide;

N-{5-[cis-3-(3-methoxy-pyridin-2-yloxy)-cyclobutyl]-1H-pyrazol-3-yl}-2-naphthalen-1-yl-acetamide;

2-methyl-cyclopropanecarboxylic acid {cis-3-[5-(2-naphthalen-1-yl-acetylamino)-2H-pyrazol-3-yl]-cyclobutyl}-amide;

2-naphthalen-1-yl-N-{5-[cis-3-(3-trifluoromethyl-pyridin-2-yloxy)-cyclobutyl]-1H-pyrazol-3-yl}-acetamide;

2-naphthalen-1-yl-N-{5-[cis-3-(3-nitro-pyridin-2-yloxy)-cyclobutyl]-1H-pyrazol-3-yl}-acetamide;

N-{5-[cis-3-(benzothiazol-2-yloxy)-cyclobutyl]-1H-pyrazol-3-yl}-2-naphthalen-1-yl-acetamide;

2-naphthalen-1-yl-N-{5-[cis-3-(4-trifluoromethyl-pyrimidin-2-yloxy)-cyclobutyl]-1H-pyrazol-3-yl}-acetamide;

2-naphthalen-1-yl-N-{5-[3-(5-nitro-pyridin-2-yloxy)-cyclobutyl]-1H-pyrazol-3-yl}-acetamide;

2-naphthalen-1-yl-N-{5-[3-(pyrimidin-2-yloxy)-cyclobutyl]-1H-pyrazol-3-yl}-acetamide;

2-naphthalen-1-yl-N-{5-[3-(5-trifluoromethyl-pyridin-2-yloxy)-cyclobutyl]-1H-pyrazol-3-yl}-acetamide;

N-{5-[3-(6-methoxy-pyridazin-3-yloxy)-cyclobutyl]-1H-pyrazol-3-yl}-2-naphthalen-1-yl-acetamide;

2-naphthalen-1-yl-N-{5-[3-(pyrazin-2-yloxy)-cyclobutyl]-1H-pyrazol-3-yl}-acetamide;

N-{5-[3-(6-methyl-pyridin-2-yloxy)-cyclobutyl]-1H-pyrazol-3-yl}-2-naphthalen-1-yl-acetamide;

N-{5-[3-(6-chloro-benzothiazol-2-yloxy)-cyclobutyl]-1H-pyrazol-3-yl}-2-naphthalen-1-yl-acetamide;

N-{5-[3-(6-methoxy-benzothiazol-2-yloxy)-cyclobutyl]-1H-pyrazol-3-yl}-2-naphthalen-1-yl-acetamide; and

pharmaceutically acceptable salts of the foregoing compounds.

Other examples of preferred compounds of formula 1 are:

N-{5-[cis-3-(4-Hydroxy-phenyl)-cyclobutyl]-1H-pyrazol-3-yl}-2-quinolin-6-yl-acetamide;

N-{5-[cis-3-(3-Hydroxy-phenyl)-cyclobutyl]-1H-pyrazol-3-yl}-2-quinolin-6-yl-acetamide;

2-Naphthalen-1-yl-N-[5-(cis-3-pyridin-3-yl-cyclobutyl)-2H-pyrazol-3-yl]-acetamide;

N-[5-(cis-3-Naphthalen-2-yl-cyclobutyl)-2H-pyrazol-3-yl]-2-pyridin-3-yl-acetamide;

N-(5-Indan-2-yl-1H-pyrazol-3-yl)-2-quinolin-6-yl-acetamide;

N-[5-(cis-3-Pyridin-2-yl-cyclobutyl)-2H-pyrazol-3-yl]-2-quinolin-6-yl-acetamide;

N-[5-(cis-3-Pyridin-2-yl-cyclobutyl)-2H-pyrazol-3-yl]-2-quinolin-6-yl-acetamide;

2-(4-Methoxy-phenyl)-N-[5-(cis-3-pyridin-4-yl-cyclobutyl)-2H-pyrazol-3-yl]-acetamide;

N-{5-[3-(cis-2-Dimethylaminomethyl-phenyl)-cyclobutyl]-2H-pyrazol-3-yl}-2-(4-methoxy-phenyl)-acetamide;

N-(5-{cis-3-[3-(2-Dimethylamino-ethoxy)-phenyl]-cyclobutyl}-2H-pyrazol-3-yl)-2-(4-methoxy-phenyl)-acetamide;

N-{5-[cis-3-(2-Hydroxy-phenyl)-cyclobutyl]-2H-pyrazol-3-yl}-2-(4-methoxy-phenyl)-acetamide;

N-(5-{cis-3-[2-(2-Dimethylamino-ethoxy)-phenyl]-cyclobutyl}-2H-pyrazol-3-yl)-2-(4-methoxy-phenyl)-acetamide;

2-(4-Methoxy-phenyl)-N-[5-(cis-3-phenyl-cyclobutyl)-2H-pyrazol-3-yl]-acetamide;

N-{5-[cis-3-(2-Fluoro-phenyl)-cyclobutyl]-2H-pyrazol-3-yl}-2-(4-methoxy-phenyl)-acetamide;

N-(5-{cis-3-[4-(Azetidin-3-yloxy)-phenyl]-cyclobutyl}-2H-pyrazol-3-yl)-2-(4-methoxy-phenyl)-acetamide;

N-(5-{cis-3-[2-(Azetidin-3-yloxy)-phenyl]-cyclobutyl}-2H-pyrazol-3-yl)-2-(4-methoxy-phenyl)-acetamide;

2-(4-Methoxy-phenyl)-N-{5-[cis-3-(2-methylsulfanyl-phenyl)-cyclobutyl-2H-pyrazol-3-yl}-acetamide;

N-{5-[cis-3-(2-Amino-phenyl)-cyclobutyl]-2H-pyrazol-3-yl}-2-(4-methoxy-phenyl)-acetamide;

N-{5-[cis-3-(4-Cyano-phenyl)-cyclobutyl]-2H-pyrazol-3-yl}-2-(4-methoxy-phenyl)-acetamide;

N-{5-{cis-3-(2-Cyano-phenyl)-3-hydroxy-cyclobutyl]-2H-pyrazol-3-yl}-2-(4-methoxy-phenyl)-acetamide;

N-{5-[cis-3-(2-Hydroxy-ethyl)-cyclobutyl]-1H-pyrazol-3-yl}-2-naphthalen-1-yl-acetamide;

N-{5-[cis-3-(3-Cyano-phenyl)-cyclobutyl]-2H-pyrazol-3-yl}-2-(4-methoxy-phenyl)-acetamide;

N-{5-[cis-3-(2-Cyano-phenyl)-cyclobutyl]-2H-pyrazol-3-yl}-2-(4-methoxy-phenyl)-acetamide;

N-{5-[cis-3-(3-Amino-phenyl)-cyclobutyl]-2H-pyrazol-3-yl}-2-(4-methoxy-phenyl)-acetamide;

4-(cis-3-{5-[2-(4-Methoxy-phenyl)-acetylamino]-1H-pyrazol-3-yl}-cyclobutyl)-benzoic acid methyl ester;

N-{5-[cis-3-(4-Hydroxymethyl-phenyl)-cyclobutyl]-2H-pyrazol-3-yl}-2-(4-methoxy-phenyl)-acetamide;

N-{5-[cis-3-(2-Hydroxy-phenyl)-cyclobutyl]-1H-pyrazol-3-yl}-2-phenyl-acetamide;

N-{5-[cis-3-(2-Hydroxy-phenyl)-cyclobutyl]-1H-pyrazol-3-yl}-2-quinolin-6-yl-acetamide;

N-{5-[cis-3-(2-Hydroxy-phenyl)-cyclobutyl]-1H-pyrazol-3-yl}-acetamide;

Cyclopropanecarboxylic acid {5-[cis-3-(2-hydroxy-phenyl)-cyclobutyl]-1H-pyrazol-3-yl}-amide;

N-{5-[cis-3-(2-Hydroxy-phenyl)-cyclobutyl]-1H-pyrazol-3-yl}-isobutyramide;

N-{5-[cis-3-(3-Aminomethyl-phenyl)-cyclobutyl]-2H-pyrazol-3-yl}-2-(4-methoxy-phenyl)-acetamide;

N-{5-[cis-3-(3-Dimethylaminomethyl-phenyl)-cyclobutyl]-2H-pyrazol-3-yl}-2-(4-methoxy-phenyl)-acetamide;

3-(cis-3-{5-[2-(4-Methoxy-phenyl)-acetylamino]-1H-pyrazol-3-yl}-cyclobutyl)-benzoic acid methyl ester;

N-{5-[cis-3-(3-Hydroxymethyl-phenyl)-cyclobutyl]-2H-pyrazol-3-yl}-2-(4-methoxy-phenyl)-acetamide;

N-{5-{cis-3-[3-(1-Hydroxy-1-methyl-ethyl)-phenyl]-cyclobutyl}-2H-pyrazol-3-yl)-2-(4-methoxy-phenyl)-acetamide;

N-{5-[cis-3-(3-Ethylaminomethyl-phenyl)-cyclobutyl]-2H-pyrazol-3-yl}-2-(4-methoxy-phenyl)-acetamide;

N-{5-[cis-3-(3-Cyclobutylaminomethyl-phenyl)-cyclobutyl]-2H-pyrazol-3-yl}-2-(4-methoxy-phenyl)-acetamide;

2-(4-Methoxy-phenyl)-N-{5-[cis-3-(3-propylaminomethyl-phenyl)-cyclobutyl]-2H-pyrazol-3-yl}-acetamide;

N-{5-[cis-3-(3-Cyclopentylaminomethyl-phenyl)-cyclobutyl]-2H-pyrazol-3-yl}-2-(4-methoxy-phenyl)-acetamide;

N-(5-{cis-3-[3-(Benzylamino-methyl)-phenyl]-cyclobutyl}-2H-pyrazol-3-yl)-2-(4-methoxy-phenyl)-acetamide;

2-(4-Methoxy-phenyl)-N-{5-[3-(3-methylaminomethyl-phenyl)-cyclobutyl]-2H-pyrazol-3-yl}-acetamide;

N-{5-[cis-3-(3-Cyclopropylaminomethyl-phenyl)-cyclobutyl]-2H-pyrazol-3-yl}-2-(4-methoxy-phenyl)-acetamide;

2-(4-Methoxy-phenyl)-N-{5-[cis-3-(3-pyrrolidin-1-ylmethyl-phenyl)-cyclobutyl]-2H-pyrazol-3-yl}-acetamide;

N-{5-[cis-3-(3-Diethylaminomethyl-phenyl)-cyclobutyl]-2H-pyrazol-3-yl}-2-(4-methoxy-phenyl)-acetamide;

N-{5-[cis-3-(3-Azetidin-1-ylmethyl-phenyl)-cyclobutyl]-2H-pyrazol-3-yl}-2-(4-methoxy-phenyl)-acetamide;

and pharmaceutically acceptable salts of the foregoing compounds.

Other specific examples of compounds of the invention of formula 1 are:

N-[5-(cis-3-pyridin-2-yl-cyclobutyl)-1H-pyrazol-3-yl]-2-quinolin-6-yl-acetamide;

N-[5-(cis-3-pyridin-3-yl-cyclobutyl)-1H-pyrazol-3-yl]-2-quinolin-6-yl-acetamide;

N-[5-(cis-3-pyridin-4-yl-cyclobutyl)-1H-pyrazol-3-yl]-2-quinolin-6-yl-acetamide;

N-[5-(cis-3-pyrazin-2-yl-cyclobutyl)-1H-pyrazol-3-yl]-2-quinolin-6-yl-acetamide;

N-[5-(cis-3-pyrimidin-4-yl-cyclobutyl)-1H-pyrazol-3-yl]-2-quinolin-6-yl-acetamide;

N-{5-[cis-3-(3-methoxy-pyridin-2-yl)-cyclobutyl]-1H-pyrazol-3-yl}-2-quinolin-6-yl-acetamide;

N-{5-[cis-3-(4-methoxy-pyridin-3-yl)-cyclobutyl]-1H-pyrazol-3-yl}-2-quinolin-6-yl-acetamide;

N-{5-[cis-3-(3-methoxy-pyridin-4-yl)-cyclobutyl]-1H-pyrazol-3-yl}-2-quinolin-6-yl-acetamide;

N-{5-[cis-3-(2-methoxy-pyridin-3-yl)-cyclobutyl]-1H-pyrazol-3-yl}-2-quinolin-6-yl-acetamide;

N-{5-[cis-3-(5-methoxy-pyrimidin-4-yl)-cyclobutyl]-1H-pyrazol-3-yl}-2-quinolin-6-yl-acetamide;

N-{5-[cis-3-(5-ethyl-1H-imidazol-2-yl)-cyclobutyl]-1H-pyrazol-3-yl}-2-quinolin-6-yl-acetamide;

N-}5-[cis-3-(1-ethyl-1H-pyrrol-2-yl)-cyclobutyl]-1H-pyrazol-3-yl}-2-quinolin-6-acetamide;

N-{5-[cis-3-(2-aminomethyl-phenyl)-cyclobutyl]-1H-pyrazol-3-yl}-2-quinolin-6-yl-acetamide;

N-{5-[cis-3-(2-pyrrolidin-1-ylmethyl-phenyl)-cyclobutyl]-1H-pyrazol-3-yl}-2-quinolin-6-yl-acetamide;

and pharmaceutically acceptable salts of the foregoing compounds.

Other specific examples of compounds of formula 1 are:

{4-[(5-cyclobutyl-1H-pyrazol-3-ylcarbamoyl)-methyl]-phenyl}-acetic acid;

N-(5-cyclobutyl-1H-pyrazol-3-yl)-2-(1H-indol-3-yl)-acetamide;

2-(3-chloro-phenyl)-N-(5-cyclobutyl-1H-pyrazol-3-yl)-acetamide;

2-(3-bromo-phenyl)-N-(5-cyclobutyl-1H-pyrazol-3-yl)-acetamide;

2-biphenyl-4-yl-N-(5-cyclobutyl-1H-pyrazol-3-yl)-acetamide;

2-biphenyl-4-yl-N-(5-cyclobutyl-1H-pyrazol-3-yl)-acetamide;

N-(5-cyclobutyl-1H-pyrazol-3-yl)-2-(3,4,5-trimethoxy-phenyl)-acetamide;

{2-[(5-cyclobutyl-1H-pyrazol-3-ylcarbamoyl)-methyl]-phenyl}-acetic acid;

N-(5-cyclobutyl-1H-pyrazol-3-yl)-2-(3,4-dichloro-phenyl)-acetamide;

2-(2-chloro-phenyl)-N-(5-cyclobutyl-1H-pyrazol-3-yl)-acetamide;

N-(5-cyclobutyl-1H-pyrazol-3-yl)-2-(2-fluoro-phenyl)-acetamide;

2-(4-butoxy-phenyl)-N-(5-cyclobutyl-1H-pyrazol-3-yl)-acetamide;

N-(5-cyclobutyl-1H-pyrazol-3-yl)-2-(2,4-difluoro-phenyl)-acetamide;

N-(5-cyclobutyl-1H-pyrazol-3-yl)-2-(2-iodo-phenyl)-acetamide;

N-(5-cyclobutyl-1H-pyrazol-3-yl)-2-(2,3-dimethoxy-phenyl)-acetamide;

N-(5-cyclobutyl-1H-pyrazol-3-yl)-2-(2,5-dihydroxy-phenyl)-acetamide;

N-(5-cyclobutyl-1H-pyrazol-3-yl)-2-(3-hydroxy-4-methoxy-phenyl)-acetamide;

2-(4-acetylamino-phenyl)-N-(5-cyclobutyl-1H-pyrazol-3-yl)-acetamide;

N-(5-cyclobutyl-1H-pyrazol-3-yl)-2-(4-trifluoromethyl-phenyl)-acetamide;

2-(4-chloro-3-nitro-phenyl)-N-(5-cyclobutyl-1H-pyrazol -3-yl)-acetamide;

N-(5-cyclobutyl-1H-pyrazol-3-yl)-2-(4-hydroxy-3,5-dinitro-phenyl)-acetamide;

N-(5-cyclobutyl-1H-pyrazol-3-yl)-2-(3,4-difluoro-phenyl)-acetamide;

2-(2,4-bis-trifluoromethyl-phenyl)-N-(5-cyclobutyl-1H-pyrazol-3-yl)-acetamide;

N-(5-cyclobutyl-1H-pyrazol-3-yl)-2-(3,5-difluoro-phenyl)-acetamide;

N-(5-cyclobutyl-1H-pyrazol-3-yl)-2-(2-fluoro-3-trifluoromethyl-phenyl)-acetamide;

N-(5-cyclobutyl-1H-pyrazol-3-yl)-2-(4-fluoro-3-trifluoromethyl-phenyl)-acetamide;

N-(5-cyclobutyl-1H-pyrazol-3-yl)-2-(2,4,6-trifluoro-phenyl)-acetamide;

N-(5-cyclobutyl-1H-pyrazol-3-yl)-2-(4-methylsulfanyl-phenyl)-acetamide;

N-(5-cyclobutyl-1H-pyrazol-3-yl)-2-(3-hydroxy-phenyl)-acetamide;

N-(5-cyclopentyl-1H-pyrazol-3-yl)-2-phenyl-acetamide;

2-(4-chloro-phenyl)-N-(5-cyclopentyl-2H-pyrazol-3-yl)-acetamide;

N-(5-cyclopentyl-1H-pyrazol-3-yl)-2-naphthalen-2-yl-acetamide;

N-(5-cyclobutyl-2H-pyrazol-3-yl)-2-(2,4-dichloro-phenyl)-acetamide;

N-(5-cyclobutyl-2H-pyrazol-3-yl)-2-quinolin-6-yl-acetamide;

2-(3-amino-phenyl)-N-(5-cyclobutyl-2H-pyrazol-3-yl)-acetamide;

1-(5-cyclobutyl-1H-pyrazol-3-yl)-3-naphthalen-1-yl-urea;

N-(5-cyclohexyl-1H-pyrazol-3-yl)-2-naphthalen-2-yl-acetamide;

N-(5-cyclohexyl-1H-pyrazol-3-yl)-2-phenyl-acetamide;

2-(4-chloro-phenyl)-N-(5-cyclohexyl-1H-pyrazol-3-yl)-acetamide;

N-(5-cyclobutyl-1H-pyrazol-3-yl)-2-(4-phenoxy-phenyl)-acetamide;

N-(5-cyclobutyl-1H-pyrazol-3-yl)-2-(4-dimethylamino-phenyl)-acetamide;

N-(5-cyclopentyl-2H-pyrazol-3-yl)-2-(2,3,4-trimethoxy-phenyl)-acetamide;

N-(5-cyclopentyl-2H-pyrazol-3-yl)-2-(4-isopropyl-phenyl)-acetamide;

N-(5-cyclopentyl-2H-pyrazol-3-yl)-2-pyrrolo[2,3-b]pyridin-1-yl-acetamide;

N-(5-cyclobutyl-1H-pyrazol-3-yl)-2-m-tolyl-acetamide;

N-(5-cyclopentyl-2H-pyrazol-3-yl)-2-p-tolyl-acetamide;

N-(5-cyclobutyl-1H-pyrazol-3-yl)-2-(3-trifluoromethoxy-phenyl)-acetamide;

N-[5-(3-benzyloxy-propyl)-1H-pyrazol-3-yl]-2-naphthalen-2-yl-acetamide;

4-[5-(2-naphthalen-2-yl-acetylamino)-1H-pyrazol-3-yl]-piperidine-1-carboxylic acid benzyl ester;

2-naphthalen-2-yl-N-(5-piperidin-4-yl-2H-pyrazol-3-yl)-acetamide;

N-[5-(1-acetyl-piperidin-4-yl)-2H-pyrazol-3-yl]-2-naphthalen-2-yl-acetamide;

N-[5-(1-benzoyl-piperidin-4-yl)-2H-pyrazol-3-yl]-2-naphthalen-2-yl-acetamide;

4-[5-(2-naphthalen-1-yl-acetylamino)-1H-pyrazol-3-yl]-piperidine-1-carboxylic acid benzyl ester;

N-[5-(1-cyclobutanecarbonyl-piperidin-4-yl)-2H-pyrazol-3-yl]-2-naphthalen-2-yl-acetamide;

N-{3-[5-(2-naphthalen-2-yl-acetylamino)-4H-pyrazol-3-yl]-propyl}-benzamide;

N-{3-[5-(2-naphthalen-1-yl-acetylamino)-2H-pyrazol-3-yl]-propyl}-benzamide;

N-{5-[1-(3-methyl-butyl)-piperidin-4-yl]-1H-pyrazol-3-yl}-2-naphthalen-2-yl-acetamide

N-[3-(5-phenylacetylamino-2H-pyrazol-3-yl)-propyl]-benzamide;

N-{3-[5-(2-m-tolyl-acetylamino)-2H-pyrazol-3-yl]-propyl}-benzamide;

N-(3-{5-[2-(3-chloro-phenyl)-acetylamino]-2H-pyrazol-3-yl}-propyl)-benzamide;

6-methyl-pyridine-2-carboxylic acid {3-[5-(2-naphthalen-2-yl-acetylamino)-1H-pyrazol-3-yl]-cyclobutyl}-amide;

6-methyl-pyridine-2-carboxylic acid {3-[5-(2-naphthalen-2-yl-acetylamino)-1H-pyrazol-3-yl]-cyclobutyl}-amide;

6-methyl-pyridine-2-carboxylic acid {3-[5-(2-naphthalen-2-yl-acetylamino)-1H-pyrazol-3-yl]-cyclobutyl}-amide;

N-{5-[3-(1,3-dioxo-1,3-dihydro-isoindol-2-yl)-cyclobutyl]-2H-pyrazol-3-yl}-2-naphthalen-2-yl-acetamide;

6-chloro-pyridine-2-carboxylic acid {3-[5-(2-naphthalen-2-yl-acetylamino)-1H-pyrazol-3-yl]-cyclobutyl}-amide;

N-{3-[5-(2-naphthalen-2-yl-acetylamino)-1H-pyrazol-3-yl]-cyclobutyl}-benzamide;

2-naphthalen-2-yl-N-[5-(2-pyridin-2-yl-ethyl)-2H-pyrazol-3-yl]-acetamide;

2-naphthalen-2-yl-N-[5-(2-pyridin-3-yl-ethyl)-2H-pyrazol-3-yl]-acetamide;

2-naphthalen-2-yl-N-[5-(2-pyridin-4-yl-ethyl)-2H-pyrazol-3-yl]-acetamide;

2-naphthalen-1-yl-N-[5-(2-pyridin-2-yl-ethyl)-2H-pyrazol-3-yl]-acetamide;

2-naphthalen-1-yl-N-[5-(2-pyridin-3-yl-ethyl)-2H-pyrazol-3-yl]-acetamide;

2-naphthalen-1-yl-N-[5-(2-pyridin-4-yl-ethyl)-2H-pyrazol-3-yl]-acetamide;

2-(3-methoxy-phenyl)-N-[5-(2-pyridin-2-yl-ethyl)-2H-pyrazol-3-yl]-acetamide;

2-(3-methoxy-phenyl)-N-[5-(2-pyridin-3-yl-ethyl)-2H-pyrazol-3-yl]-acetamide;

2-(3-methoxy-phenyl)-N-[5-(2-pyridin-4-yl-ethyl)-2H-pyrazol-3-yl]-acetamide;

2-(3-methoxy-phenyl)-N-[5-(2-thiazol-2-yl-ethyl)-2H-pyrazol-3-yl]-acetamide;

2-naphthalen-1-yl-N-[5-(2-thiazol-2-yl-ethyl)-2H-pyrazol-3-yl]-acetamide;

2-naphthalen-2-yl-N-[5-(2-thiazol-2-yl-ethyl)-2H-pyrazol-3-yl]-acetamide;

N-[5-(1-benzyl-piperidin-4-yl)-1H-pyrazol-3-yl]-2-naphthalen-2-yl-acetamide;

2-naphthalen-1-yl-N-(5-piperidin-4-yl-1H-pyrazol-3-yl)-acetamide;

2-(4-chloro-phenyl)-N-{3-[5-(2-naphthalen-2-yl-acetylamino)-1H-pyrazol-3-yl]-cyclobutyl}-acetamide;

pyrazine-2-carboxylic acid {3-[5-(2-naphthalen-2-yl-acetylamino)-1H-pyrazol-3-yl]-cyclobutyl}-amide;

2-(3-methoxy-phenyl)-N-{5-[2-(2-trifluoromethyl-phenyl)-ethyl]-2H-pyrazol-3-yl}-acetamide;

2-(3-methoxy-phenyl)-N-{5-[2-(3-trifluoromethyl-phenyl)-ethyl]-2H-pyrazol-3-yl}-acetamide;

2-(3-methoxy-phenyl)-N-{5-[2-(4-trifluoromethyl-phenyl)-ethyl]-2H-pyrazol-3-yl}-acetamide;

6-methyl-pyridine-2-carboxylic acid (3-{5-[2-(3-methoxy-phenyl)-acetylamino]-2H-pyrazol-3-yl}-cyclobutyl)-amide;

6-methyl-pyridine-2-carboxylic acid (3-{5-[2-(4-methoxy-phenyl)-acetylamino]-2H-pyrazol-3-yl}-cyclobutyl)-amide;

6-methyl-pyridine-2-carboxylic acid (3-{5-[2-(4-chloro-phenyl)-acetylamino]-2H-pyrazol-3-yl}-cyclobutyl)-amide; and

pharmaceutically acceptable salts of said compounds.

Salts of compounds of formula 1 can be obtained by forming salts with any acidic or basic group present on a compound of formula 1. Examples of pharmaceutically acceptable salts of the compounds of formula 1 are the salts of hydrochloric acid, p-toluenesulfonic acid, fumaric acid, citric acid, succinic acid, salicylic acid, oxalic acid, hydrobromic acid, phosphoric acid, methanesulfonic acid, tartaric acid, maleic acid, di-p-toluoyl tartaric acid, acetic acid, sulfuric acid, hydroiodic acid, mandelic acid, sodium, potassium, magnesium, calcium, and lithium.

The compounds of formula 1 may have optical centers and therefore may occur in different enantiomeric and other stereoisomeric configurations. The invention includes all enantiomers, diastereomers, and other stereoisomers of such compounds of formula 1, as well as racemic and other mixtures thereof.

The subject invention also includes isotopically-labeled compounds, which are identical to those recited in formula 1, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ³H, ¹¹C, ¹⁴C, ¹⁵F, ¹²³I and ¹²⁵I. Compounds of the present invention and pharmaceutically acceptable salts of said compounds that contain the aforementioned isotopes and/or other isotopes of other atoms are within the scope of this invention. Isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography), and 125I isotopes are particularly useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula 1 of this invention can generally be prepared by carrying out the procedures disclosed in the Schemes and/or in the Examples below, by substituting a readily available isotopically labeled reagent for a non-isotopically labeled reagent.

This invention also includes compounds of the formula

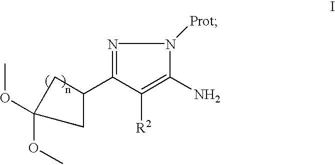

wherein Prot is a protecting group;

R² is H, F, —CH₃, —CN, or —C(═O)OR⁷;

and n is an integer selected from 1, 2, 3, and 4.

Compounds of formula I are useful as intermediates for synthesizing certain compounds of formula 1 that are described herein.

Preferably, n is 1.

Examples of specific protecting groups include, but are not limited to t-butyl and —CH₂— Ar, wherein “Ar” is an aryl or heteroaryl group. An example of the latter type of protecting group is para-methoxybenzyl.

This invention also provides a pharmaceutical composition for treating a disease or condition comprising abnormal cell growth in a mammal, including a human, comprising a compound of formula 1 in an amount effective in inhibiting abnormal cell growth, and a pharmaceutically acceptable carrier.

This invention also provides a pharmaceutical composition for treating a disease or condition comprising abnormal cell growth in a mammal, including a human, comprising a compound of formula 1 in an amount effective to inhibit cdk2 activity, and a pharmaceutically acceptable carrier.

This invention also provides a method for treating a disease or condition comprising abnormal cell growth in a mammal, including a human, comprising administering to the mammal a compound of formula 1 in an amount effective in inhibiting abnormal cell growth.

This invention also provides a method for treating a diseases or condition comprising abnormal cell growth in a mammal, including a human, comprising administering to the mammal a compound of formula 1 in an amount effective to inhibit cdk2 activity.

In a pharmaceutical composition or method of this invention for treating a disease or condition comprising abnormal cell growth, the disease or condition comprising abnormal cell growth is in one embodiment cancer. The cancer may be a carcinoma, for example carcinoma of the bladder, breast, colon, kidney, liver, lung, for example small cell lung cancer, esophagus, gall bladder, ovary, pancreas, stomach, cervix, thyroid, prostate, or skin, for example squamous cell carcinoma; a hematopoietic tumor of lymphoid lineage, for example leukemia, acute lymphocytic leukemia, B-cell lymphoma, T-cell lymphoma, Hodgkins lymphoma, non-Hodgkins lymphoma, hairy cell lymphoma, or Burkett's lymphoma; a hematopoietic tumor of myeloid lineage, for example acute and chronic myelogenous leukemias, myelodysplastic syndrome, or promyelocytic leukemia; a tumor of mesenchymal origin, for example fibrosarcoma or rhabdomyosarcoma; a tumor of the central or peripheral nervous system, for example astrocytoma, neuroblastoma, glioma or schwannoma; melanoma; seminoma; teratocarcinoma; osteosarcoma; xenoderoma pigmentoum; keratoctanthoma; thyroid follicular cancer; or Kaposi's sarcoma.

In another embodiment, the disease or condition comprising abnormal cell growth is benign. Such diseases and conditions include benign prostate hyperplasia, familial adenomatosis polyposis, neuro-fibromatosis, atherosclerosis, pulmonary fibrosis, arthritis, psoriasis, glomerulonephritis, restenosis, hypertrophic scar formation, inflammatory bowel disease, transplantation rejection, fungal infection, and endotoxic shock.

This invention also provides a pharmaceutical composition for treating a neurodegenerative disease or condition in a mammal, including a human, comprising a compound of formula 1 in an amount effective in treating said disease or condition, and a pharmaceutically acceptable carrier.

This invention also provides a pharmaceutical composition for treating a neurodegenerative disease or condition in a mammal, including a human, comprising a compound of formula 1 in an amount effective in inhibiting cdk5 activity, and a pharmaceutically acceptable carrier.

This invention also provides a method for treating a neurodegnerative disease or condition in a mammal, including a human, comprising administering to the mammal a compound of formula 1 in an amount effective in inhibiting cdk5 activity.

This invention also provides a method for treating a neurodegenerative disease or condition in a mammal, including a human, comprising administering to the mammal a compound of formula 1 in an amount effective in treating said disease or condition.

In one embodiment of the invention, the neurodegenerative disease or condition which is treated is selected from Huntington's disease, stroke, spinal cord trauma, traumatic brain injury, multiinfarct dementia, epilepsy, amyotrophic lateral sclerosis, pain, viral induced dementia for example AIDS induced dementia, neurodegeneration associated with bacterial infection, migraine, hypoglycemia, urinary incontinece, brain ischemia, multiple sclerosis, Alzheimer's disease, senile dementia of the Alzheimer's type, mild cognitive impairment, age-related cognitive decline, emesis, corticobasal degeneration, dementia pugilistica, Down's syndrome, myotonic dystrophy, Niemann-Pick disease, Pick's disease, prion disease with tangles, progessive supranuclear palsy, lower lateral sclerosis, and subacute sclerosing panencephalistis.

This invention also provides a pharmaceutical composition for treating a disease or condition the treatment of which can be effected or facilitated by altering dopamine mediated neurotransmission in a mammal, including a human, comprising a compound of formula 1 in an amount effective in treating said disease or condition and a pharmaceutically acceptable carrier.

This invention also provides a pharmaceutical composition for treating a disease or condition the treatment of which can be effected or facilitated by altering dopamine mediated neurotransmission in a mammal, including a human, comprising a compound of formula 1 in an amount effective to inhibit cdk5 and a pharmaceutically acceptable carrier.

This invention also provides a method for treating a disease or condition the treatment of which can be effected or facilitated by altering dopamine mediated neurotransmission in a mammal, including a human, comprising administering to the mammal a compound of formula 1 in an amount effective in inhibiting cdk5 activity.

This invention also provides a method for treating a disease or condition the treatment of which can be effected or facilitated by altering dopamine mediated neurotransmission in a mammal, including a human, comprising administering to the mammal a compound of formula 1 in an amount effective in treating said disease or condition.

In one embodiment of the invention, the disease or condition the treatment of which can be effected or facilitated by altering dopamine mediated neurotransmission is selected from Parkinson's disease; schizophrenia; schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; personality disorder of the schizold type; drug addiction, including narcotic (e.g. heroin, opium, and morphine), cocaine and alcohol addiction; drug withdrawal, including narcotic, cocaine and alcohol withdrawal; obsessive compulsive disorder; Tourette's syndrome; depression; a major depressive episode, a manic or mixed mood episode, a hypomanic mood episode, a depressive episode with atypical features or with melancholic features or catatonic features, a mood episode with postpartum onset; post-stroke depression, major depressive disorder, dysthymic disorder, minor depressive disorder, premenstrual dysphoric disorder, post-psychotic depressive disorder of schizophrenia, a major depressive disorder superimposed on a psychotic disorder such as delusional disorder or schizophrenia, a bipolar disorder, for example bipolar I disorder, bipolar II disorder, cyclothymic disorder; anxiety; attention deficit and hyperactivity disorder; and attention deficit disorder.

This invention also provides a pharmaceutical composition for treating a disease or condition the treatment of which can be effected or facilitated by decreasing cdk5 activity in a mammal, including a human, which composition comprises a compound of formula 1 in an amount effective in inhibiting cdk5 activity and a pharmaceutically acceptable carrier.

This invention also provides a method for treating a disease or condition the treatment of which can be effected or facilitated by decreasing cdk5 activity in a mammal, including a human, which method comprises administering to the mammal a compound of formula 1 in an amount effective in inhibiting cdk5 activity.

We have also found that the compounds of formula 1 have activity in inhibiting GSK-3. The compounds of formula 1 therefore can be expected to be useful in treating diseases and conditions the treatment of which can be effected or facilitated by inhibition of GSK-3. Diseases and conditions the treatment of which can be effected or facilitated by inhibiting GSK-3 include neurodegenerative diseases and conditions. Neurodegenerative diseases and conditions are discussed above and include, but are not limited to, for example Alzheimer's disease, Parkinson's disease, Huntington's disease, amyotrophic lateral sclerosis, multiple sclerosis, stroke, cerebral ischemia, AIDS-related dementia, neurodegeneration associated with bacterial infection, multiinfarct dementia, traumatic brain injury, and spinal cord trauma. Therefore, compounds of formula 1 are effective in treating neurodegenerative diseases and conditions based on both cdk5 activity and GSK-3 activity.

Other diseases and conditions the treatment of which can be effected or facilitated by inhibiting GSK-3 include psychotic disorders and conditions, for example schizophrenia, schizophreniform disorder; schizoaffective disorder, for example of the delusional type or the depressive type; delusional disorder; substance-induced psychotic disorder, for example psychosis induced by alcohol, amphetamine, cannabis, cocaine, hallucinogens, inhalants, opioids, or phencyclidine; personality disorder of the paranoid type; and personality disorder of the schizold type. The treatment of such diseases and conditions can also be effected or facilitated by altering dopamine mediated neurotransmission. Therefore, compounds of formula 1 are effective in treating such disorders and conditions based on both cdk5 activity and GSK-3 activity.

Other disorders and conditions the treatment of which can be effected or facilitated by inhibiting GSK-3 include mood disorders and mood episodes, for example a major depressive episode, a manic or mixed mood episode, a hypomanic mood episode, a depressive episode with atypical features or with melancholic features or catatonic features, a mood episode with postpartum onset; post-stroke depression, major depressive disorder, dysthymic disorder, minor depressive disorder, premenstrual dysphoric disorder, post-psychotic depressive disorder of schizophrenia, a major depressive disorder superimposed on a psychotic disorder such as delusional disorder or schizophrenia, a bipolar disorder, for example bipolar I disorder, bipolar II disorder, and cyclothymic disorder. The treatment of such mood disorders and episodes, for example depression, can also be effected or facilitated by altering dopamine mediated neurotransmission. Therefore, compounds of formula 1 are effective in treating certain mood disorders and mood episodes based on both cdk5 activity and GSK-3 activity.

Other disorders and conditions the treatment of which can be effected or facilitated by inhibiting GSK-3 are male fertility and sperm motility; diabetes mellitus; impaired glucose tolerance; metabolic syndrome or syndrome X; polycystic ovary syndrome; adipogenesis and obesity; myogenesis and frailty, for example age-related decline in physical performance; acute sarcopenia, for example muscle atrophy and/or cachexia associated with burns, bed rest, limb immobilization, or major thoracic, abdominal, and/or orthopedic surgery; sepsis; spinal cord injury; hair loss, hair thinning, and balding; immunodeficiency; and cancer.

Accordingly, the present invention also provides a pharmaceutical composition for treating in a mammal, including a human, a disease or condition selected from male fertility and sperm motility; diabetes mellitus; impaired glucose tolerance; metabolic syndrome or syndrome X; polycystic ovary syndrome; adipogenesis and obesity; myogenesis and frailty, for example age-related decline in physical performance; acute sarcopenia, for example muscle atrophy and/or cachexia associated with burns, bed rest, limb immobilization, or major thoracic, abdominal, and/or orthopedic surgery; sepsis; hair loss, hair thinning, and balding; and immunodeficiency; which composition comprises a pharmaceutically acceptable carrier and an amount of a compound of formula 1 effective in treating said disease or condition.

The present invention further provides a pharmaceutical composition for treating in a mammal, including a human, a disease or condition selected from male fertility and sperm motility; diabetes mellitus; impaired glucose tolerance; metabolic syndrome or syndrome X; polycystic ovary syndrome; adipogenesis and obesity; myogenesis and frailty, for example age-related decline in physical performance; acute sarcopenia, for example muscle atrophy and/or cachexia associated with burns, bed rest, limb immobilization, or major thoracic, abdominal, and/or orthopedic surgery; sepsis; hair loss, hair thinning, and balding; and immunodeficiency; which composition comprises a pharmaceutically acceptable carrier and an amount of a compound of formula 1 effective in inhibiting GSK-3.

The present invention also provides a method for treating in a mammal, including a human, a disease or condition selected from male fertility and sperm motility; diabetes mellitus; impaired glucose tolerance; metabolic syndrome or syndrome X; polycystic ovary syndrome; adipogenesis and obesity; myogenesis and frailty, for example age-related decline in physical performance; acute sarcopenia, for example muscle atrophy and/or cachexia associated with burns, bed rest, limb immobilization, or major thoracic, abdominal, and/or orthopedic surgery; sepsis; hair loss, hair thinning, and balding; and immunodeficiency; which method comprises administering to said mammal an amount of a compound of formula 1 effective in treating said disease or condition.

The present invention also provides a method for treating in a mammal, including a human, a disease or condition selected from male fertility and sperm motility; diabetes mellitus; impaired glucose tolerance; metabolic syndrome or syndrome X; polycystic ovary syndrome; adipogenesis and obesity; myogenesis and frailty, for example age-related decline in physical performance; acute sarcopenia, for example muscle atrophy and/or cachexia associated with burns, bed rest, limb immobilization, or major thoracic, abdominal, and/or orthopedic surgery; sepsis; hair loss, hair thinning, and balding; and immunodeficiency; which method comprises administering to said mammal an amount of a compound of formula 1 effective in inhibiting GSK-3.

The present invention further provides a method for inhibiting GSK-3 in a mammal, including a human, which method comprises administering to said mammal an amount of a compound of formula 1 effective in inhibiting GSK-3.

The present invention further provides a pharmaceutical composition for treating in a mammal, including a human, a disorder selected from Alzheimer's disease, mild cognitive impairment, and age-related cognitive decline comprising a compound of formula 1 and a COX-II inhibitor together in an amount effective in treating said disorder, and a pharmaceutically acceptable carrier.

This invention also provides a method for treating in a mammal, including a human, a disorder selected from Alzheimer's disease, mild cognitive impariment, and age-related cognitive decline which method comprises administering to said mammal a compound of formula 1 and a COX-II inhibitor, wherein the combined amounts of the compound of formula 1 and the COX-II inhibitor are effective in treating said disorder. The compound of formula 1 and the COX-II inhibitor can be administered to the mammal at the same time and/or separately. Moreover, the compound of formula 1 and the COX-II inhibitor can be administered in a single composition or in separate compositions.

Moreover, a compound of formula 1 of the invention, or a pharmaceutically acceptable salt of a compound of formula 1, can be administered or formulated into a pharmaceutical composition with one or more anti-depressants or anxiolytic compounds for treatment or prevention of depression and/or anxiety.

Accordingly, this invention also provides a pharmaceutical composition for treating depression or anxiety in a mammal, including a human, comprising a compound of formula 1 and an NK-1 receptor antagonist together in an amount effective in treating depression or anxiety, and a pharmaceutically acceptable carrier.

This invention further provides a method for treating depression or anxiety in a mammal, including a human, which method comprises administering to said mammal a compound of formula 1 and an NK-1 receptor antagonist, wherein the combined amounts of the compound of formula 1 and the NK-1 receptor antagonist are effective in treating depression or anxiety. The compound of formula 1 and the NK-1 receptor antagonist can be administered to the mammal at the same time and/or at different times. Moreover, the may be administered together in a single pharmaceutical composition or in separate compositions.

This invention also provides a pharmaceutical composition for treating depression or anxiety in a mammal, including a human, comprising a compound of formula 1 and a 5HT_{1D} receptor antagonist together in an amount effective in treating depression or anxiety, and a pharmaceutically acceptable carrier.

This invention further provides a method for treating depression or anxiety in a mammal, including a human, which method comprises administering to said mammal a compound of formula 1 and a 5HT_{1D} receptor antagonist, wherein the combined amounts of the compound of formula 1 and the 5HT_{1D} receptor antagonist are effective in treating depression or anxiety. The compound of formula 1 and the 5HT_{1D} receptor antagonist can be administered to the mammal at the same time and/or at different times. Moreover, they may be administered together in a single pharmaceutical composition or in separate compositions.

This invention also provides a pharmaceutical composition for treating depression or anxiety in a mammal, including a human, comprising a compound of formula 1 and a SSRI together in an amount effective in treating depression or anxiety, and a pharmaceutically acceptable carrier.

This invention further provides a method for treating depression or anxiety in a mammal, including a human, which method comprises administering to said mammal a compound of formula 1 and a SSRI, wherein the combined amounts of the compound of formula 1 and the SSRI are effective in treating depression or anxiety. The compound of formula 1 and the SSRI can be administered to the mammal at the same time and/or at different times. Moreover, they may be administered together in a single pharmaceutical composition or in separate pharmaceutical compositions.

This invention also provides a pharmaceutical composition for treating schizophrenia in a mammal, including a human, comprising a compound of formula 1 and an antipsychotic selected from ziprasidone, olanzapine, risperidone, L-745870, sonepiprazole, RP 62203, NGD 941, balaperidone, flesinoxan, and gepirone, together in an amount effective in treating schizophrenia, and a pharmaceutically acceptable carrier.

This invention further provides a method for treating schizophrenia in a mammal, including a human, which method comprises administering to said mammal a compound of formula 1 and an antipsychotic selected from ziprasidone, olanzapine, risperidone, L-745870, sonepiprazole, RP 62203, NGD 941, balaperidone, flesinoxan, and gepirone, wherein the combined amounts of the compound of formula 1 and the antipsychotic are effective in treating schizophrenia. The compound of formula 1 and the antipsychotic can be administered to the mammal at the same time and/or at different times. Moreover, they may be administered together in a single pharmaceutical composition or in separate pharmaceutical compositions.

This invention also provides a pharmaceutical composition for treating a disorder selected from Alzheimer's disease, mild cognitive impairment, and age-related cognitive decline in a mammal, including a human, comprising a compound of formula 1 and an acetylcholinesterase inhibitor together in an amount effective in treating said disorder, and a pharmaceutically acceptable carrier.

This invention further provides a method for treating in a mammal, including a human, a disorder selected from Alzheimer's disease, mild cognitive impairment, and age-related cognitive decline, which method comprises administering to said mammal a compound of formula 1 and an acetylcholinesterase inhibitor, wherein the combined amounts of the compound of formula 1 and the acetylcholinesterase inhibitor are effective in treating said disorder. The compound of formula 1 and the acetylcholinesterase inhibitor can be administered to the mammal at the same time and/or at different times. Moreover, they may be administered together in a single pharmaceutical composition or in separate pharmaceutical compositions.

This invention also provides a pharmaceutical composition for treating a disease or condition selected from stroke, spinal cord trauma, traumatic brain injury, multiinfarct dementia, epilepsy, pain, Alzheimer's disease, and senile dementia comprising a compound of formula 1 and TPA (tissue plasminogen activator, for example ACTIVASE) together in an amount effective in treating said disorder, and a pharmaceutically acceptable carrier.

This invention further provides a method for treating in a mammal, including a human, a disease or condition selected from stroke, spinal cord trauma, traumatic brain injury, multiinfarct dementia, epilepsy, pain, Alzheimer's disease, and senile dementia, which method comprises administering to said mammal a compound of formula 1 and TPA, wherein the combined amounts of the compound of formula 1 and the TPA are effective in treating said disease or condition. The compound of formula 1 and the TPA can be administered to the mammal at the same time and/or at different times. Moreover, they may be administered together in a single pharmaceutical composition or in separate pharmaceutical compositions.

This invention also provides a pharmaceutical composition for treating a disease or condition selected from stroke, spinal cord trauma, traumatic brain injury, multiinfarct dementia, epilepsy, pain, Alzheimer's disease, and senile dementia in a mammal, including a human, comprising a compound of formula 1 and NIF (neutrophil inhibitory factor) together in an amount effective in treating said disorder, and a pharmaceutically acceptable carrier.

This invention further provides a method for treating in a mammal, including a human, a disease or condition selected from stroke, spinal cord trauma, traumatic brain injury, multiinfarct dementia, epilepsy, pain, Alzheimer's disease, and senile dementia, which method comprises administering to said mammal a compound of formula 1 and NIF, wherein the combined amounts of the compound of formula 1 and the NIF are effective in treating said disease or condition. The compound of formula 1 and the NIF can be administered to the mammal at the same time and/or at different times. Moreover, they may be administered together in a single pharmaceutical composition or in separate pharmaceutical compositions.

This invention also provides a pharmaceutical composition for treating a disease or condition selected from Huntington's disease, stroke, spinal cord trauma, traumatic brain injury, multiinfarct dementia, epilepsy, amyotrophic lateral sclerosis, pain, viral induced dementia for example AIDS induced dementia, migraine, hypoglycemia, urinary incontinece, brain ischemia, multiple sclerosis, Alzheimer's disease, senile dementia of the Alzheimer's type, mild cognitive impairment, age-related cognitive decline, emesis, corticobasal degeneration, dementia pugilistica, Down's syndrome, myotonic dystrophy, Niemann-Pick disease, Pick's disease, prion disease with tangles, progessive supranuclear palsy, lower lateral sclerosis, and subacute sclerosing panencephalistis in a mammal, including a human, comprising a compound of formula 1 and an NMDA receptor antagonist together in an amount effective in treating said disorder, and a pharmaceutically acceptable carrier.

This invention further provides a method for treating in a mammal, including a human, a disease or condition selected from Huntington's disease, stroke, spinal cord trauma, traumatic brain injury, multiinfarct dementia, epilepsy, amyotrophic lateral sclerosis, pain, viral induced dementia for example AIDS induced dementia, migraine, hypoglycemia, urinary incontinece, brain ischemia, multiple sclerosis, Alzheimer's disease, senile dementia of the Alzheimer's type, mild cognitive impairment, age-related cognitive decline, emesis, corticobasal degeneration, dementia pugilistica, Down's syndrome, myotonic dystrophy, Niemann-Pick disease, Pick's disease, prion disease with tangles, progessive supranuclear palsy, lower lateral sclerosis, and subacute sclerosing panencephalistis, which method comprises administering to said mammal a compound of formula 1 and an NMDA receptor antagonist, wherein the combined amounts of the compound of formula 1 and the NMDA receptor antagonist are effective in treating said disease or condition. The compound of formula 1 and the NMDA receptor antagonist can be administered to the mammal at the same time and/or at different times. Moreover, they may be administered together in a single pharmaceutical composition or in separate pharmaceutical compositions.

This invention also provides a pharmaceutical composition for treating a disease or condition selected from stroke, spinal cord trauma, traumatic brain injury, multiinfarct dementia, epilepsy, pain, Alzheimer's disease, and senile dementia in a mammal, including a human, comprising a compound of formula 1 and a potassium channel modulator together in an amount effective in treating said disorder, and a pharmaceutically acceptable carrier.

This invention further provides a method for treating in a mammal, including a human, a disease or condition selected from stroke, spinal cord trauma, traumatic brain injury, multiinfarct dementia, epilepsy, pain, Alzheimer's disease, and senile dementia, which method comprises administering to said mammal a compound of formula 1 and a potassium channel modulator, wherein the combined amounts of the compound of formula 1 and the potassium channel modulator are effective in treating said disease or condition. The compound of formula 1 and the potassium channel modulator can be administered to the mammal at the same time and/or at different times. Moreover, they may be administered together in a single pharmaceutical composition or in separate pharmaceutical compositions.

The terms “treatment”, “treating”, and the like, refers to reversing, alleviating, or inhibiting the progress of the disease or condition to which such term applies, or one or more symptoms of such disease or condition. As used herein, these terms also encompass, depending on the condition of the patient, preventing the onset of a disease or condition or of symptoms associated with a disease or condition, including reducing the severity of a disease or condition or symptoms associated therewith prior to affliction with said disease or condition. Such prevention or reduction prior to affliction refers to administration of the compound of the invention to a subject that is not at the time of administration afflicted with the disease or condition. “Preventing” also encompasses preventing the recurrence of a disease or condition or of symptoms associated therewith.

“Abnormal cell growth”, as used herein, refers to cell growth, either malignant (e.g. as in cancer) or benign, that is independent of normal regulatory mechanisms (e.g., loss of contact inhibition). Examples of benign proliferative diseases are psoriasis, benign prostatic hypertrophy, human papilloma virus (HPV), and restinosis.

“Neurodegenerative diseases and conditions”, as used herein and unless otherwise indicated, refers to diseases and conditions having associated therewith degeneration of neurons. Conditions and diseases that are neurodegenerative in nature are generally known to those of ordinary skill in the art.

References herein to diseases and conditions “the treatment of which can be effected or facilitated by altering dopamine mediated neurotransmission” mean a disease or condition that is caused at least in part by dopamine neurotransmission, or a disease or condition that result in abnormal dopamine neurotransmission, thus contributing to symptoms or manifestations of the disease condition.

References herein to diseases and conditions “the treatment of which can be effected or facilitated by decreasing cdk5 activity” mean a disease or condition that is caused at least in part by cdk5 activity, or a disease or condition that results in abnormal cdk5 activity that contributes to symptoms or manifestations of the disease or condition.

An “amount effective to inhibit cdk5” as used herein refers to an amount of a compound sufficient to bind to the enzyme cdk5 with the effect of decreasing cdk5 activity.

An “amount effective to inhibit cdk2 activity” as used herein refers to an amount of a compound sufficient to bind to the enzyme cdk2 with the effect of decreasing cdk2 activity.

### DETAILED DESCRIPTION OF THE INVENTION

Compounds of the formula 1, above, and their pharmaceutically acceptable salts, can be prepared according to the following reaction Schemes and discussion. Unless otherwise indicated R¹, R², R³, and R⁴ are as defined above. “Prot” represents a protecting group. Isolation and purification of the products is accomplished by standard procedures which are known to a chemist of ordinary skill.

As used herein, the expression “reaction inert solvent” refers to a solvent system in which the components do not interact with starting materials, reagents, or intermediates of products in a manner which adversely affects the yield of the desired product.

During any of the following synthetic sequences it may be necessary and/or desirable to protect sensitive or reactive groups on any of the molecules concerned. This may be achieved by means of conventional protecting groups, such as those described in T. W. Greene, Protective Groups in Organic Chemistry, John Wiley & Sons, 1981; and T. W. Greene and P. G. M. Wuts, Protective Groups in Organic Chemistry, John Wiley & Sons, Inc., 1999.

Scheme 1 illustrates general methods suitable for preparing compounds of formula 1 wherein R³ is —(CR¹⁰R¹¹)ₙ—, —C(═O)NR⁹—, —C(═O)O—, or —C(═O)(CR¹⁰R¹¹)ₙ—. Treatment of a solution of slurry of magnesium chloride in a reaction inert solvent, preferably acetonitrile, diethyl ether, or tetrahydrofuran, at a reaction temperature from −20° C. to 40° C., preferably from about −5° C. to 21° C., with a trialkyl amine base, where triethyl amine or diisopropylethylamine are preferred, in the presence of an alkyl cyano acetate and in the presence of an acid halide of formula 2, wherein acid chlorides are preferred, affords intermediate 3a, a 2-cyano-3-alkyl-3-oxo-propionic acid alkyl ester. A preferred alkyl cyano acetate is ethyl cyano acetate. Hydrolysis and decarboxylation of 3a to 3 may be accomplished by exposing 3a to water in a reaction inert solvent, preferably dimethylsulfoxide, at a temperature from about 21° C. to 200° C., preferably from about 100° C. to 118° C. Reaction of 3 in a reaction inert solvent, such as a lower alcohol, in the presence of a hydrazine at a reaction temperature of from about 0° C. to about 150° C., where a temperature of from about 70° C. to about 85° C. is preferred, affords the corresponding product 4. The hydrazine used may be anhydrous hydrazine or a hydrate form of hydrazine, or N-alkyl-hydrazine or N-acyl-hydrazine. Anhydrous hydrazine or an alkyl hydrazine, for example 4-methoxy-benzyl-hydrazine, are preferred.

Coupling of 4 with an aryl halide or heteroaryl halide to obtain an intermediate of formula 5, wherein R³ is —(CR¹⁰R¹¹)₀— (a bond) can be accomplished by reaction of 4 in a reaction inert solvent, preferably toluene, at a reaction temperature of from about 21° C. to about 150° C., preferably at about 100° C. to about 110° C., in the presence of a palladium catalyst, a base, preferably cesium carbonate or sodium or potassium tert-butoxide, a ligand, where preferred ligands are 2,2′-bis(diphenylphosphino)-1,1′-binaphythyl, 2-(dicyclohexylphosphino)biphenyl, and 2-(di-tert-butylphosphino)biphenyl, and in the presence of the appropriate aryl halide or heteroaryl halide, where aryl bromides or chlorides and heteroaryl bromides or chlorides are preferred. The metal catalyst may be a palladium species, for example palladium chloride, palladium acetate, dichlorobis(acetonitrile)palladium, or derivatives thereof, wherein palladium acetate is preferred. Removal of the protecting group from 5 can be accomplished by reaction of 5 in a reaction inert solvent, preferably methylene chloride or no solvent, in the presence of an acid, wherein trifluoro acetic acid is preferred, at a reaction temperature from about 20° C. to about 100° C., preferably from about 65° C. to about 75° C., to yield 1a, wherein R³ represents a bond and R⁴ is as defined above for compounds of formula 1.

Coupling of 4 to yield N-acyl derivatives of the nature 6 (wherein R³ is —C(═O)(CR¹⁰R¹¹)ₙ—) can be accomplished by reaction of 4 in a reaction inert solvent, preferably methylene chloride, pyridine, tetrahydrofuran, or diethyl ether, in the presence of an acid chloride ClC(═O)(CH₂)ₙR⁴, acid anhydride R⁴(CH₂)ₙC(═O))₂O, or an activated carboxylic acid derivative XC(═O)(CH₂)ₙR⁴ wherein X represents the activating group, and in the presence of a amine base, such as triethyl amine or diisopropylethyl amine, wherein tripropylphosphonic anhydride and triethylamine are a preferred combination, at a temperature of from about −78° C. to about 40° C. The activated carboxylic acid derivative can be prepared from the carboxylic acid and known activating reagents such as polymer supported coupling agents or coupling agents such as, for instance, dicyclohexyl carbodiimide, carbonyl diimidazole, tripropylphosphonic anhydride, alkyl chloroformate, bis(2-oxo-3-oxazolidinyl)phosphinic chloride, benzotriazol-1-yloxy-tris(dimethylamino)phosphonium hexafluorophosphate, or any other such standard literature reagents. Removal of the protecting group on 6 may be accomplished by reaction 6 in a reaction inert solvent, wherein methylene chloride or no solvent is preferred, in the presence of an acid, wherein trifluoro acetic acid is preferred, at a reaction temperature from about 20° C. to about 100° C., preferably from about 65° C. to about 75° C. A product of formula 1b is obtained, wherein R³ represents —C(═O)(CR¹⁰R¹¹)ₙ— and R⁴ is as defined above for compounds of formula 1.

Alternatively, the amine of 4 can be treated with a base, such as triethylamine, diisopropylethylamine, pyridine, or 2,6-lutidine, and an alkyl, aryl, or heteroaryl chloroformate ClC(═O)OR⁴ (diisopropylethylamine and aryl or heteroarly chloroformates are a preferred combination) from a temperature about −78° C. to about 40° C., to afford a compound of an intermediate where R³—C(═O)O— and R⁴ is as defined above for compounds of formula 1. Removal of the protecting group from this intermediate can be achieved as described, i.e. in a reaction inert solvent, wherein methylene chloride or no solvent is preferred, in the presence of an acid, wherein trifluoro acetic acid is preferred, at a reaction temperature from about 20° C. to about 100° C., preferably from about 65° C. to about 75° C. This affords a carbamate compound of formula 1 wherein R³ is —C(═O)O— and R⁴ is as defined above for compounds of formula 1.

Subsequent treatment of the carbamate of the formula 1 formed in the preceding paragraph with a primary or secondary amine in a solvent such as dioxane, dimethylformamide, or acetonitrile, where a 1:1 mixture of dioxane: dimethylformamide is preferred, at a temperature between about 40° C. and about 90° C., where about 70° C. is preferred, affords the corresponding urea product of formula 1 where R³ is —C(═O)NR⁹— and R⁴ is as defined above for compounds of formula 1.

Compounds of formula 1 wherein R³ is —(CR¹⁰R¹¹)₍₁₋₃₎— can be prepared from intermediates of formula 4 by reaction of 4 with an oxo moiety (aldehyde or ketone) in a reaction inert solvent, preferably toluene, tetrahydrofuran or methanol at a reaction temperature from about 0° C. to about 110° C., preferably about 21° C., in the presence of a reducing ragent, where preferred reducing reagents are sodium triacetoxyborohydride, sodium cyanoborohydride, and lithium aluminum hydride to afford an intermediate of formula 6 wherein R³ is —(CR¹⁰R¹¹)₍₁₋₃₎—. Removal of the protecting group from this intermediate 6 can be achieved as described, i.e. in a reaction inert solvent, wherein trifluoro acetic acid is preferred, at a reaction temperature from about 20° C. to about 100° C., preferably from about 65° C. to about 75° C.

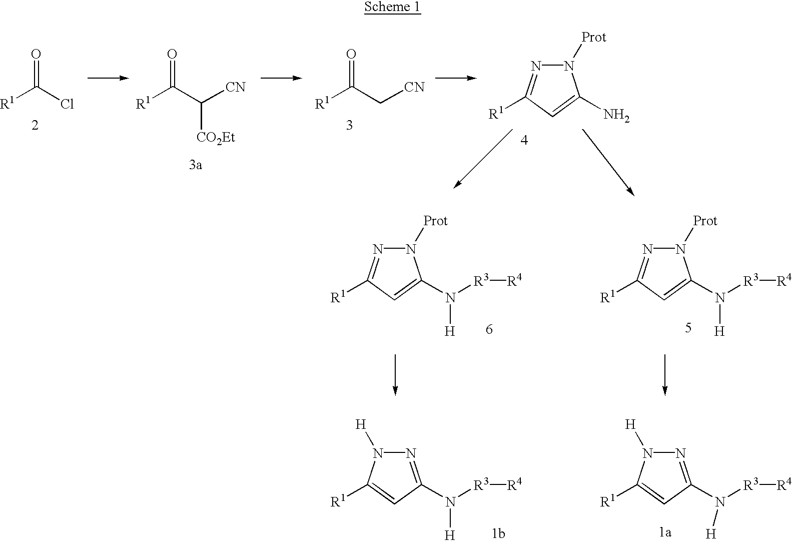

Scheme 2 illustrates an alternative method suitable for preparing compounds of formula 1. The method depicted in Scheme 2 is preferred when R⁴ is an electron deficient aryl moiety, such as 4-nitrophenyl, or an electron deficient heteroaryl moiety. Reaction of a ketone having the general structure of 8, wherein R¹ and R² are as defined above for compounds of formula 1, in a reaction inert solvent, wherein tetrahydrofuran or diethyl ether are preferred as solvents, at a reaction temperature from about −116° C. to about 50° C., preferably at about −78° C. to about −65° C., in the presence of a base, a hindered amine base being preferred, and also in the presence of an isothiocyanate of the general formula 9, wherein R³ is a bond (—(CR¹⁰R¹¹)₀—) and R⁴ is aryl or heteroaryl, affords 10. Examples of hindered amine bases include lithium diisopropyl amide, potassium bis(trimethylsilyl) amide, lithium bis(trimethylsilyl) amide, and other such standard literature reagents. Treatment of 10 in a reaction inert solvent, a preferred solvent being a lower alcohol, in the presence of an acid, preferably acetic acid, at a reaction temperature of from about 21° C. to about 100° C., preferably from about 75° C. to about 85° C., and in the presence of hydrazine, affords a compound of the formula 1a, wherein R³ is a bond and R⁴ is aryl or heteroaryl.

Synthesis of compounds of formula 1 wherein R¹ is substituted with one or more substituents R⁵ is also illustrated in Scheme 2. Reaction of a compound of formula 10 in a reaction inert solvent, such as lower alcohols, in the presence of an acid, preferably acetic acid, also in the presence of a hydrazine, at a reaction temperature of about 0° C. to about 150° C., preferably from about 75° C. to about 85° C., affords 10a (wherein R³ is a bond, R⁴ is aryl or heteroaryl, R¹ is as defined above, and R⁵ is a protected oxo moiety (an acetal or ketal).

Preferred hydrazines are alkyl hydrazines, for example 4-methoxy-benzyl hydrazine or t-butyl hydrazine.

Deprotection of the oxo moiety R⁵ can be accomplished using well-known conditions, which appear in the literature. For example, treating compound 10a in a reaction inert solvent, preferably a lower ketone for example acetone, in the presence of an acid, preferably hydrogen chloride, p-toluenesulfonic acid monohydrate, or pyridinium p-toluenesulfonate, at a temperature of from about room temperature to about 80° C., preferably about 75° C., affords 10b, wherein R⁵ is an oxo (carbonyl) moiety, R¹ is as defined above, R³ is a bond, and R⁴ is aryl or heteroaryl.

Reduction of the oxo moiety to obtain an alocohol (R⁵ is —OH) can be accomplished using well established chemistry. Alternatively, the oxo moiety of 10b can be reacted with an amine, either primary or secondary, wherein the preferred amine is an alkyl amine for example 4-methoxy-benzyl-amine, in a reaction inert solvent, preferably toluene or tetrahydrofuran, at a reaction temperature from about 21° C. to about 150° C., preferably at about 70° C. to about 110° C. After 10b is consumed, usually within a 12 hour period, the reaction is cooled to a temperature of about 21° C. to about 50° C. and a reducing reagent is added, where preferred reducing reagents are sodium triacetoxyborohydride, sodium cyanoborohydride and lithium aluminum hydride, to afford 10c, wherein R¹ is as defined above, R³ is a bond, R⁴ is aryl or heteroaryl, and R⁵ is —NR⁷R⁸. Coupling of 10c to yield N-acyl derivatives of the formula 10d, wherein R⁵ is —NR⁷C(═O)R⁸, can be accomplished by reaction of 10c in a reaction inert solvent, wherein methylene chloride, pyridine, tetrahydrofuran, diethyl ether are preferred, in the presence of an alkyl chloroformate, acid chloride, acid anhydride, or an activated carboxylic acid derivative, from −78° C. to 40° C. The activated carboxylic acid derivative is prepared from the carboxylic acid and known activating reagents such as polymer supported coupling agents or alternatively dicyclohexyl carbodiimide, carbonyl diimidazole, tripropylphosphonic anhydride, alkyl chloroformate, bis(2-oxo-3-oxazolidinyl)phosphinic chloride, benzotriazol-1-yloxy-tris(dimethylamino)phosphonium hexafluorophosphate, or any other such standard literature reagents, in the presence of a trialkyl amine base, such as triethyl amine or diisopropylethyl amine, wherein tripropylphosphonic anhydride and triethylamine are a preferred combination.

Removal of the protecting group on 10b, 10c, or 10d may be accomplished by reaction in a reaction inert solvent, wherein methylene chloride or no solvent is preferred, in the presence of an acid, preferably trifluoro acetic acid, at a reaction temperature from about 20° C. to about 100° C., preferably from about 65° C. to about 75° C., to yield the corresponding compounds of formula 1, for example 1c, as depicted in Scheme 2.

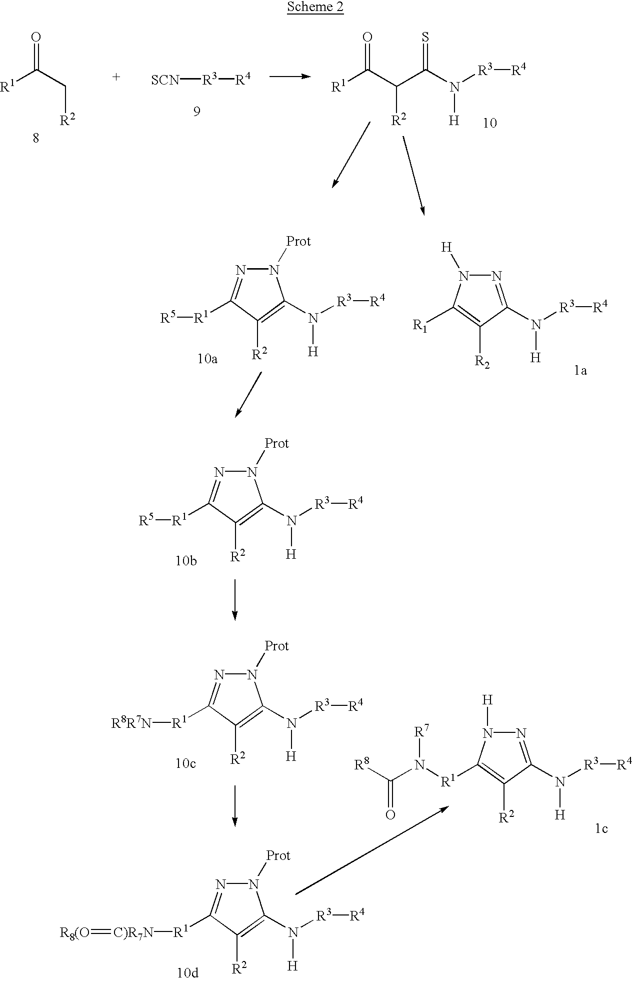

Scheme 3 illustrates an alternative method suitable for the preparation of compounds of formula 1. The method illustrated in Scheme 3 is preferred when R¹ is substituted with R⁵ selected from —NR⁷R⁸, —NR⁷C(═O)R⁸, —NR⁷C(═O)NR⁸R⁹, —NR⁷S(═O)₂R⁸, —NR⁷S(═O)₂NR⁸R⁹, —C(═O)R⁷, —C(═O)OR⁷, and —C(═O)NR⁷R⁸. Referring to Scheme 3, reacting an organolithium base, preferably n-butyl lithium, sec-butyl lithium, phenyl lithium, or tert-butyl lithium, at a reaction temperature from about −116° C. to about 50° C., preferably at about −78° C. to about −45° C., in a reaction inert solvent, especially tetrahydrofuran or diethyl ether, in the presence of alkylnitrile, preferably acetonitrile, and in the presence of an ester of the formula 11, wherein R¹ is as defined above and R⁵ is a protected oxo moiety (specifically a ketal or acetal) or R⁵ is as defined above, gives 3b, wherein R⁵ is the same as in the compound of formula 11. Processing of a compound with the formula 3b to a compound with the formula 1d or formula 1e where R³ is, respectively, —C(═O)(R¹⁰R¹¹)ₙ— or a bond and R⁵ is as in the compound of formula 11 can be accomplished as described in the description of Scheme 1. If R⁵ is a protected oxo moiety, conversion of such group to carbonyl, can be accomplished at the same time as removal of the protecting group from the pyrazole nitrogen.

Alternatively, the amine of 4a can be treated with a base, such as triethylamine, diisopropylethylamine, pyridine, or 2,6-lutidine, and a chloroformate ClC(═O)OR⁴ (diisopropylethylamine and aryl or heteroarly chloroformates are a preferred combination) as described above for scheme 1 to afford a carbamate intermediate 7a where R³—C(═O)O— and R⁴ is as defined above for compounds of formula 1.

Subsequent treatment of 7a formed in the preceding paragraph with a primary or secondary amine in a solvent such as dioxane, dimethylformamide, or acetonitrile, where a 1:1 mixture of dioxane: dimethylformamide is preferred, at a temperature between about 40° C. and about 90° C., where about 70° C. is preferred, affords the corresponding urea intermediate 7b where R³ is —C(═O)NR⁹— and R⁴ is as defined above for compounds of formula 1.

Intermediate 7c wherein R³ is —(CR¹⁰OR¹¹)₍₁₋₃₎— can be prepared from intermediate 4a by reaction of 4a with an oxo moiety (aldehyde or ketone) in a reaction inert solvent, preferably toluene, tetrahydrofuran or methanol at a reaction temperature from about 0° C. to about 110° C., preferably about 21° C., in the presence of a reducing reagent, where preferred reducing reagents are sodium triacetoxyborohydride, sodium cyanoborohydride, and lithium aluminum hydride to afford an intermediate of formula 6a wherein R³ is —(CR¹⁰OR¹¹)₍₁₋₃₎—. Removal of the protecting group from this intermediate 6a can be achieved as described, i.e. in a reaction inert solvent, wherein trifluoro acetic acid is preferred, at a reaction temperature from about 20° C. to about 100° C., preferably from about 65° C. to about 75° C.

Intermediates of formula 5a, 6a, 7a, 7b, or 7c can also be converted to form further compounds of the invention, as illustrated in Scheme 3. Scheme 3 depicts use of 5a (wherein R³ is a bond) and 6a (wherein R³ is —C(═O)(R¹⁰R¹¹)ₙ—) as reactants, however the same chemistry can be applied to intermediates of formulae 7a, 7b, or 7c to obtain analogous products. Referring to Scheme 3, if R⁵ in 5a and 6a is a protected oxo (carbonyl) moiety (specifically acetal or ketal), removal of the protecting group can first be accomplished using well-known conditions, which appear in the literature. Treating a compound Sa or 6a (or 7a, 7b, or 7c), in a reaction inert solvent, preferably a lower ketone, for example acetone, in the presence of an acid, where preferred acids are hydrogen chloride, p-toluenesulfonic acid monohydrate, pyridinium p-toluenesulfonate, at a temperature varying from about room temperature to about 80° C., preferable at about 75° C., affords 12, where R³ is a bond, or 15, where R³ is —C(═O)(CR¹⁰R¹¹R)ₙ—, or, if 7a, 7b, or 7c were used as reactants, compounds analogous to 12 and 15 but wherein R³ is —C(═O)O—, —C(═O)NR⁹—, or —(CR¹⁰R¹¹)₍₁₋₃₎—, respectively, in each case R⁵ being an oxo (carbonyl) moiety.

The oxo moiety of the intermediates produced in the preceding paragraph, such as in 12 and 15, can be reacted with an amine, primary or secondary, wherein the preferred amines are alkyl amine for example 4-methoxy-benzyl-amine, in a reaction inert solvent, preferably toluene or tetrahydrofuran, at a reaction temperature from about 21° C. to about 150° C., preferably at about 70° C. to about 110° C. After 12 or 15 is consumed, usually within a 12 hour period, the reaction can be cooled to a temperature of about 21° C. to about 50° C., and a reducing reagent is added. Preferred reducing reagents are sodium triacetoxyborohydride, sodium cyanoborohydride and lithium aluminum hydride. Such reaction of for example 12 or 15 affords 13, wherein R³ is a bond, or 16, wherein R³ is —C(═O)(CR¹⁰R¹¹)ₙ—, respectively, in each case R⁵ being —NR⁷R⁸. Analogous compounds to 13 and 16, wherein R⁵ is —NR⁷R⁸ and R³ is —C(═O)O—, —C(═O)NR⁹—, or —(CR¹⁰R¹¹)₍₁₋₃₎— can also be prepared, using the intermediates analogous to 12 and 15 described above. Coupling of 13 or 16 or the analogous intermediates to yield an N-acyl derivative 14 or 17 or N-acyl derivatives analogous thereto but having R³ as —C(═O)O—, —C(═O)NR⁹—, or —(CR¹⁰R¹¹)₍₁₋₃₎—, can be accomplished by reaction of 13 or 16 or the analogous intermediates in a reaction inert solvent, wherein methylene chloride, pyridine, tetrahydrofuran, diethyl ether are preferred, in the presence of an acid chloride, acid anhydride, or an activated carboxylic acid derivative, from about −78° C. to about 40° C. The activated carboxylic acid can be prepared as described above.

Alternatively, the amine of 16 or 13 or the intermediates analogous thereto can be treated with a base, such as triethylamine, diisopropylethylamine, pyridine, or 2,6-lutidine, and an alkyl, aryl or heteroaryl chloroformate ClC(═O)₂R⁷ (diisopropylethylamine and chloroformates are a preferred combination) from a temperature of about −78° C. to about 40° C., where from about −78° C. to about −40° C. are preferred, to afford a compounds of formula 1 where R⁵—NR⁷C(═O)OR⁸. Subsequent treatment of the carbamate of the formula 1 with a primary or secondary amine in a solvent such as dioxane, dimethylformamide, or acetonitrile, where a 1:1 mixture of dioxane: dimethylformamide is preferred, at a temperature between 40° C. and 90° C., where 70° C. is preferred, affords the corresponding urea intermediate where R⁵ is —NR⁷C(═O)NR⁸R⁹.

Removal of the protecting group on 12, 13, 14, 15, 16, or 17 or any of the analogous compounds described above wherein R³ and/or R⁵ is instead —C(═O)O—, —C(═O)NR⁹—, or —(CR¹⁰OR¹¹)₍₁₋₃₎— may be accomplished by reaction in a reaction inert solvent, wherein methylene chloride or no solvent is preferred, in the presence of an acid, wherein trifluoro acetic acid is preferred, at a reaction temperature from about 20° C. to about 100° C., preferably from about 65° C. to about 75° C., affording respective compounds of formula 1, for example a compound formula 1c, wherein R³ is a bond and R⁵ is —NR⁷C(═O)R⁸ or a compound of formula 1f, wherein R³ is —C(═O)(CR¹⁰R¹¹)ₙ— and R⁵ is —NR⁷C(═O)R⁸.

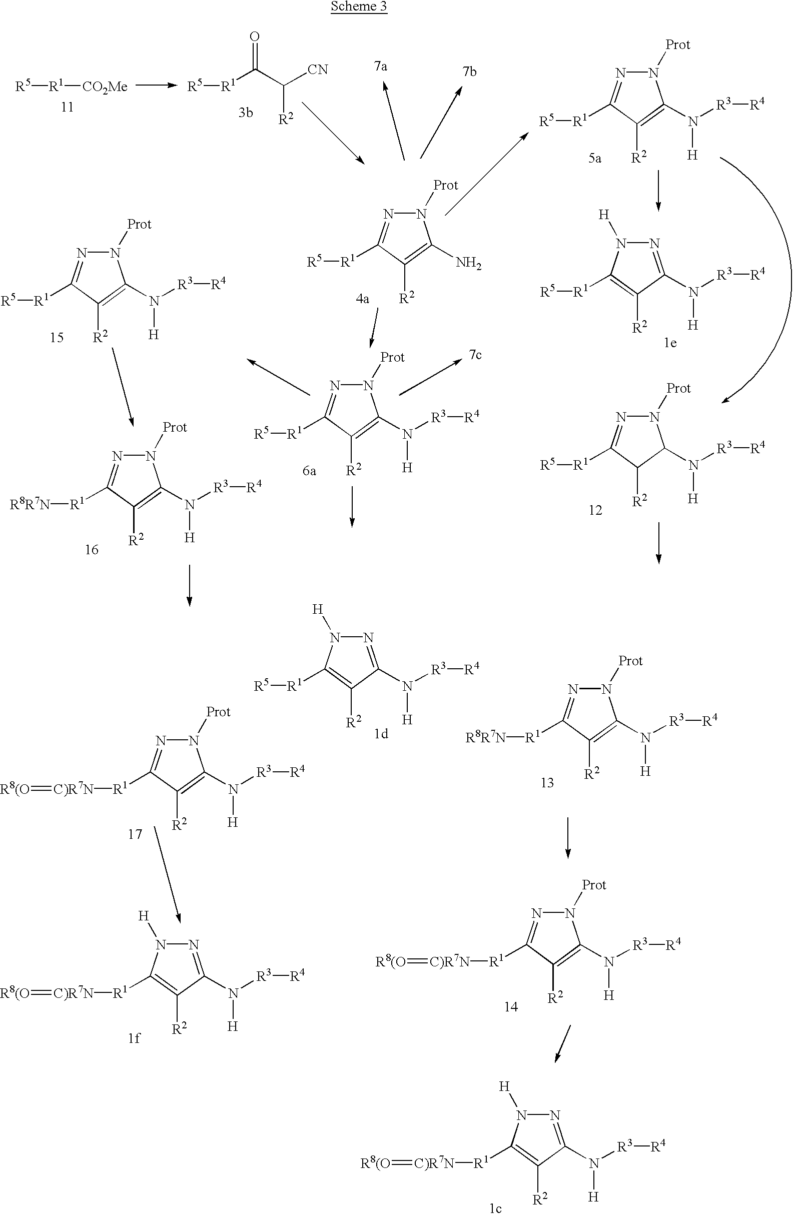

Schemes 4 and 5 illustrate a preferred method for the preparation of compounds of the formula 1, wherein R¹ is optionally substituted with OR⁷ or R⁷. Preparation of the intermediates 12 and 15 can be accomplished as described in the description of Scheme 3. R³ in intermediate 12 is a bond, and R³ in intermediate 15 is —C(═O)(CR¹⁰R¹¹)ₙ—. Also, intermediates analogous to 12 and 15, but wherein R³ is —C(═O)O—, —C(═O)NR⁹—, or —(CR¹⁰R¹¹)₍₁₋₃₎—, can be used in Schemes 4 and 5. In each case (12, 15, or analogous intermediate), R⁵ is an oxo (carbonyl) moiety.

Conversion of the oxo (═O) moiety to a hydroxyl moiety (—OH), as in 18 and 23, can be accomplished using well-established chemistry. The preferred method is by reaction of 12 or 15 or intermediate analogous thereto in a reaction inert solvent, preferably a tetrahydrofuran/water mixture, at a reaction temperature from about −78° C. to about 50° C., preferable at about 20° C., in the presence of a reducing agent, preferably NaBH₄ or lithium aluminum hydride, to afford 18 or 23 or compound analogous thereto wherein R³ is —C(═O)O—, —C(═O)NR⁹—, or —(CR¹⁰R¹¹)₍₁₋₃₎—.

Reaction of 18 or 23 or compound analogous thereto wherein R³ is —C(═O)O—, —C(═O)NR⁹—, or —(CR¹⁰OR¹¹)₍₁₋₃₎— in a reaction inert solvent, preferably tetrahydrofuran, at a reaction temperature from about −20° C. to about 50° C., preferably at about 20° C., in the presence of R⁷-halide, where the preferred R⁷-halide is R⁷-Cl, affords 19 or 24 or compound analogous thereto wherein R³ is —C(═O)O—, —C(═O)NR⁹—, or —(CR¹⁰R¹¹)₍₁₋₃₎—, in each case R⁵ being —OR⁷. Removal of the protecting group can be accomplished by reaction in a reaction inert solvent, wherein methylene chloride or no solvent is preferred, in the presents of an acid, wherein trifluoro acetic acid is preferred, at a reaction temperature from about 20° C. to 100° C., preferably from about 65° C. to about 75° C., to obtain a compound of formula 1g, wherein R³ is a bond and where R⁵=OR⁷, a compound of formula 1h, wherein R³ is —C(═O)(CR¹⁰OR¹¹)₍₀₋₃—, or an analogous compound wherein R³ is —C(═O)O—, —C(═O)NR⁹—, or —(CR¹⁰R¹¹)₍₁₋₃₎—.

Alternatively, treatment of a solution of compound 12 or 15 or analogous intermediate wherein R³ is —C(═O)O—, —C(═O)NR⁹—, or —(CR¹⁰R¹¹)₍₁₋₃₎— (in each case R⁵ being an oxo moiety, specifically carbonyl) in a reaction inert solvent, where tetrahydrofuran is preferred, with an organometallic reagent, such as an organomagnesium halide, organolithium, organocerium, organotitanium, organozinc, organocopper, or organoaluminum, where the organomagnesium halide (Grignard reagent) or organolithium reagents are preferred, at a temperature from about −78° C. to about 40° C., where −78° C. to about 0° C. is preferred, affords the alcohol product 21 or 26 or analogous compound wherein R³ is —C(═O)O—, —C(═O)NR⁹—, or —(CR¹⁰R¹¹)₍₁₋₃₎—, wherein in each case R¹ is hydroxylated and substituted with R⁷, for example (5-(3-hydroxy, 3-phenyl-cyclobutyl)-2-(Prot)-pyrazol-3-yl)-naphthalen-2-yl-amine. Removal of the alcohol is accomplished by reaction of 21 or 26 or analogous compound in an inert solvent, such as methylene chloride, chloroform, or 1,2-dichloroethane, or preferably no solvent, in the presence of an acid, preferably trifluoroacetic acid, and in the presence of a silane, where triethylsilane and triphenylsilane are preferred, at a temperature from about −10° C. to about 50° C., where about 20° C. to about 40° C. is preferred, obtaining a compound of formula 22 or 27 or compound analogous thereto but wherein R³ is —C(═O)O—, —C(═O)NR⁹—, or —(CR¹⁰R¹¹)₍₁₋₃₎—, the intermediate in each case comprising R⁷ bonded directly to R¹.

Alternatively, treatment of the alcohol products such as 21 or 26 and about one to ten equivalents of a base, where about five equivalents of pyridine or 2,6-lutidine are preferred, in a reaction inert solvent, preferably methylene chloride, with a strongly acidic reagant, preferably thionyl chloride, at a temperature from about −110° C. to about 0° C., where about −78° C. to about −45° C. is preferred, results in replacement of the alcohol (—OH group) with chloride. Reductive removal of the chloride may be accomplished by exposing a mixture of said chloride of 21 or 26 and a noble metal catalyst, palladium being preferred, to an atmosphere of hydrogen gas at a pressure of about 1 to about 100 atmospheres, where a preferred pressure of hydrogen gas is about one to about ten atmospheres, to obtain 22 or 27 or a compound analogous thereto wherein R³ is —C(═O)O—, —C(═O)NR—, or —(CR¹⁰R¹¹)₍₁₋₃₎—. The metal catalyst may be conveniently suspended on an inert solid support such as charcoal, in a solvent such as ethyl acetate, tetrahydrofuran, dioxane, or a mixture thereof.

In Schemes 4 and 5, the R⁵ hydroxy moiety of a compound of formula 18 or 23, or of the analogous compounds wherein R³ is —C(═O)O—, —C(═O)NR⁹—, or —(CR¹⁰R¹¹)₍₁₋₃₎—, can be derivatized using chemistry known in the art to obtain corresponding compounds wherein R⁵ is —OC(═O)R⁷, —OC(═O)OR⁷, —OC(═O)NR⁷R⁸, and —OC(═O)SR⁷, —SR⁷, —S(═O)R⁷, —S(═O)₂R⁷, or —S(═O)₂NR⁷R⁸.

For example, the amine of 18 or 23 or a compound analogous thereto wherein R³ is —C(═O)O—, —C(═O)NR⁹—, or —(CR¹⁰R¹¹)₍₁₋₃₎—, can be treated with a base, such as triethylamine, diisopropylethylamine, pyridine, or 2,6-lutidine, and an alkyl, aryl or heteroaryl chloroformate ClC(═O)₂R⁷ (diisopropylethylamine and chloroformates are a preferred combination) from a temperature of about −78° C. to about 40° C., where from about −78° C. to −40° C. are preferred, to afford a carbonate intermediate where R⁵ is —OC(═O)OR⁸. Subsequent treatment of the carbonate with a primary or secondary amine in a solvent such as dioxane, dimethylformamide, or acetonitrile, where a 1:1 mixture of dioxane: dimethylformamide is preferred, at a temperature between about 40° C. and about 90° C., where about 70° C. is preferred, affords the corresponding urea intermediate where R⁵ is —OC(═O)NR⁸R⁹. Removal of the protecting group of either intermediate may be accomplished by reaction in a reaction of inert solvent, wherein methylene chloride or no solvent is preferred, in the presence of an acid, wherein trifluoro acetic acid is preferred, at a reaction temperature from about 20° C. to about 100° C., preferably from about 65° C. to about 75° C., affording a compound of formula 1, where R⁵—OC(═O)OR⁷ or —OC(═O)NR⁸R⁹.

Removal of the protecting group of 22, 27, or a derivative of 19 or 24 can be accomplished by reaction in a reaction inert solvent, preferably methylene chloride, chloroform, 1,2-dichloroethane, or no solvent, in the presence of an acid, where trifluoroacetic acid is preferred, at a reaction temperature from about 20° C. to about 100° C., preferably from about 65° C. to about 75° C., affording a compound of formula 1i, 1k, or other compound of formula 1.

Compounds of formula 1 as described herein wherein R² is other than hydrogen can be prepared by transformations of the compounds of formula 1 herein wherein R² is hydrogen using methods that are well known in the art. For example, referring to Scheme 3, supra, compounds of formula 1 wherein R² is F can be prepared by treating compounds of formula 17, 6a, or 14 with N-fluorobenzenesulfonimide in a reaction of inert solvent, wherein toluene, dioxane, or xylenes are preferred, from about room temperature to about 150° C., preferably from about 100° C. to about 120° C. to obtain the corresponding intermediates wherein R² is F. Removal of the protecting group from these intermediates may be accomplished by reaction in a reaction of inert solvent, wherein methylene chloride or no solvent are preferred, in the presence of an acid, wherein trifluoro acetic acid is preferred, at a reaction temperature from about 20° C. to about 100° C., preferably from about 65° C. to about 75° C., thus affording compounds of formula 1 wherein R² is F.

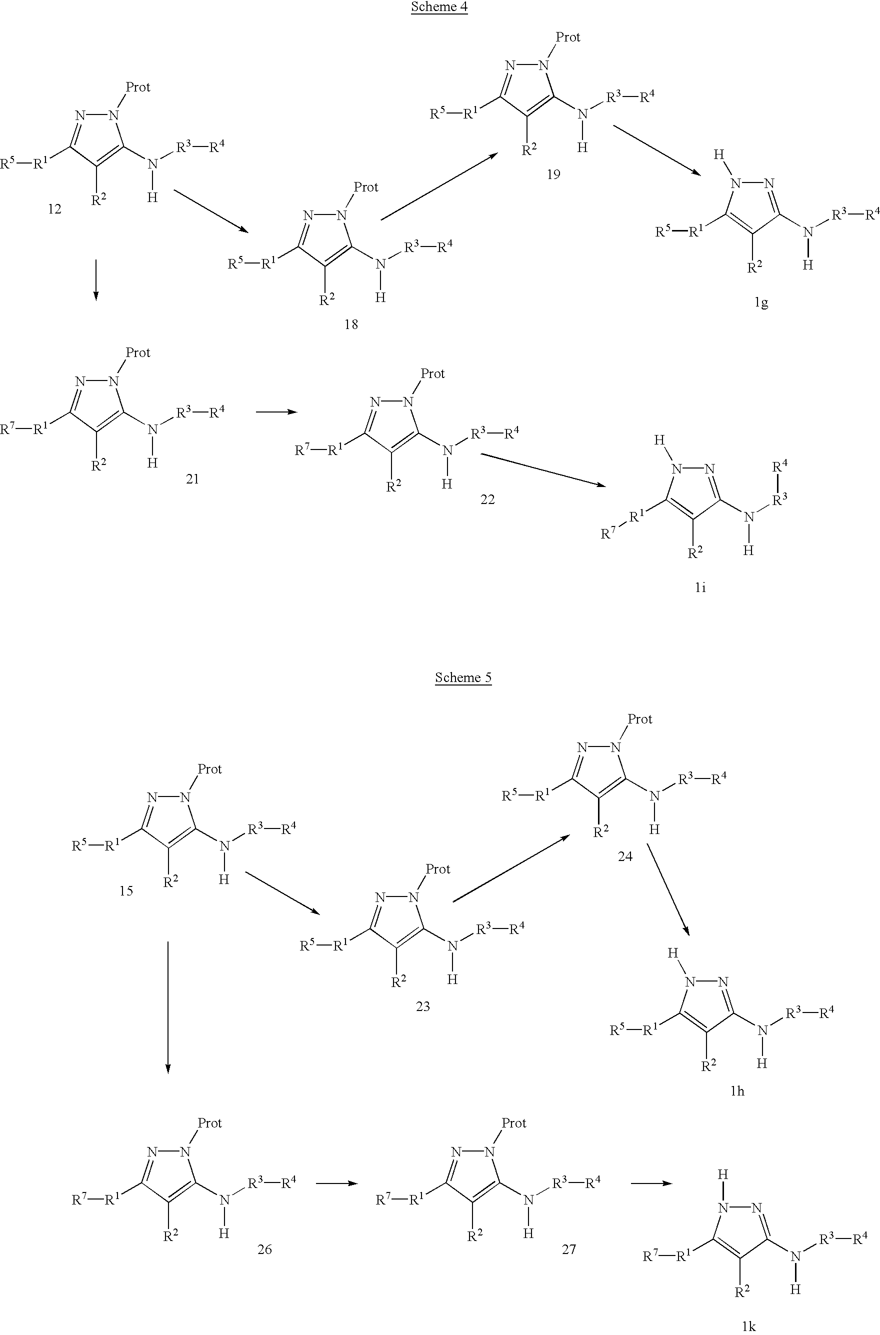

Some of the compounds of formula 1 prepared according to the above processes are obtained as a mixture of isomers or enantiomers. Such mixtures of isomers or enantiomers are within the scope of the present invention. The separation of such mixtures into the single isomers or enantiomers according to conventional techniques is also within the scope of the present invention, as are the separated isomers and enantiomers themselves.

Pharmaceutically acceptable salts of a compound of formula 1 can be prepared in a conventional manner by treating a solution or suspension of the corresponding free base or acid with one chemical equivalent of a pharmaceutically acceptable acid or base. Conventional concentration or crystallization techniques can be employed to isolate the salts. Illustrative of suitable acids are acetic, lactic, succinic, maleic, tartaric, citric, gluconic, ascorbic, benzoic, cinnamic, fumaric, sulfuric, phosphoric, hydrochloric, hydrobromic, hydroiodic, sulfamic, sulfonic acids such as methanesulfonic, benzene sulfonic, p-toluenesulfonic, and related acids. Illustrative bases are sodium, potassium, and calcium.

A compound of this invention may be administered alone or in combination with pharmaceutically acceptable carriers, in either single or multiple doses. Suitable pharmaceutical carriers include inert solid diluents or fillers, sterile aqueous solutions and various organic solvents. The pharmaceutical compositions formed by combining a compound of formula 1 or a pharmaceutically acceptable salt thereof can then be readily administered in a variety of dosage forms such as tablets, powders, lozenges, syrups, injectable solutions and the like. These pharmaceutical compositions can, if desired, contain additional ingredients such as flavorings, binders, excipients and the like. Thus, for purposes of oral administration, tablets containing various excipients such as sodium citrate, calcium carbonate and calcium phosphate may be employed along with various disintegrants such as starch, methylcellulose, alginic acid and certain complex silicates, together with binding agents such as polyvinylpyrrolidone, sucrose, gelatin and acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the essential active ingredient therein may be combined with various sweetening or flavoring agents, coloring matter or dyes and, if desired, emulsifying or suspending agents, together with diluents such as water, ethanol, propylene glycol, glycerin and combinations thereof.

For parenteral administration, solutions containing a compound of this invention or a pharmaceutically acceptable salt thereof in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. The sterile aqueous media employed are all readily available by standard techniques known to those skilled in the art.

A compound of formula 1 or a pharmaceutically acceptable salt thereof can be administered orally, transdermally (e.g., through the use of a patch), parenterally (e.g. intravenously), rectally, or topically. In general, the daily dosage for treating a neurodegenerative disease or condition or a disease or condition the treatment of which can be effected or facilitated by altering dopamine mediated neurotransmission will generally range from about 0.0001 to about 10.0 mg/kg body weight of the patient to be treated. The daily dosage for treating cancer or disease or condition involving abnormal cell growth of a benign nature will generally range from about 0.0001 to about 500 mg/kg body weight of the patient to be treated. As an example, a compound of the formula 1 or a pharmaceutically acceptable salt thereof can be administered for treatment of a neurodegenerative disorder to an adult human of average weight (about 70 kg) in a dose ranging from about 0.01 mg up to about 1000 mg per day, preferably from about 0.1 to about 500 mg per day, in single or divided (i.e., multiple) portions. Variations based on the aforementioned dosage ranges may be made by a physician of ordinary skill taking into account known considerations such as the weight, age, and condition of the person being treated, the severity of the affliction, and the particular route of administration chosen.

The compounds of formula 1 and their pharmaceutically acceptable salts can furthermore also be administered or formulated into a pharmaceutical composition with an amount of one or more substances selected from anti-angiogenesis agents, signal transduction inhibitors, and antiproliferative agents, which amounts are together effective in inhibiting abnormal cell growth.

Anti-angiogenesis agents, such as MMP-2 (matrix-metalloproteinase 2) inhibitors, MMP-9 (matrix-metalloproteinase 9) inhibitors, and COX-II (cyclooxygenase II) inhibitors, can be used in conjunction with a compound of formula 1 in the methods and pharmaceutical compositions described herein for treatment of abnormal cell growth, including cancer. Examples of useful COX-II inhibitors include CELEBREX™ (celecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13,1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), United States Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in the present invention are AG-3340, RO 32-3555, RS 13-0830, and the compounds recited in the following list:

3-[[4-(4-fluoro-phenoxy)-benzenesulfonyl]-(1-hydroxycarbamoyl-cyclopentyl)-amino]-propionic acid;

3-exo-3-[4-(4-fluoro-phenoxy)-benzenesulfonylamino]-8-oxa-bicyclo[3.2.1]octane-3-carboxylic acid hydroxyamide;

(2R, 3R) 1-[4-(2-chloro-4-fluoro-benzyloxy)-benzenesulfonyl]-3-hydroxy-3-methyl-piperidine-2-carboxylic acid hydroxyamide;

4-[4-(4-fluoro-phenoxy)-benzenesulfonylamino]-tetrahydro-pyran-4-carboxylic acid hydroxyamide;

3-[[4-(4-fluoro-phenoxy)-benzenesulfonyl]-(1-hydroxycarbamoyl-cyclobutyl)-amino]-propionic acid;

4-[4-(4-chloro-phenoxy)-benzenesulfonylamino]-tetrahydro-pyran-4-carboxylic acid hydroxyamide;

(R) 3-[4-(4-chloro-phenoxy)-benzenesulfonylamino]-tetrahydro-pyran-3-carboxylic acid hydroxyamide;

(2R, 3R) 1-[4-(4-fluoro-2-methyl-benzyloxy)-benzenesulfonyl]-3-hydroxy-3-methyl-piperidine-2-carboxylic acid hydroxyamide;

3-[[4-(4-fluoro-phenoxy)-benzenesulfonyl]-(1-hydroxycarbamoyl-1-methyl-ethyl)-amino]-propionic acid;

3-[[4-(4-fluoro-phenoxy)-benzenesulfonyl]-(4-hydroxycarbamoyl-tetrahydro-pyran-4-yl)-amino]-propionic acid;

3-exo-3-[4-(4-chloro-phenoxy)-benzenesulfonylamino]-8-oxa-bicyclo[3.2.1]octane-3-carboxylic acid hydroxyamide;

3-endo-3-[4-(4-fluoro-phenoxy)-benzenesulfonylamino]-8-oxa-bicyclo[3.2.1 ]octane-3-carboxylic acid hydroxyamide; and

(R) 3-[4-(4-fluoro-phenoxy)-benzenesulfonylamino]-tetrahydro-furan-3-carboxylic acid hydroxyamide;

and pharmaceutically acceptable salts and solvates of said compounds.

Other anti-angiogenesis agents, including other COX-II inhibitors and other MMP inhibitors, can also be used in the present invention.

The effective amount of a COX-II inhibitor in combination with a compound of formula 1 can generally be determined by a person of ordinary skill. A proposed daily effective dose range for a COX-II inhibitor in combination with a cdk5 inhibitor is from about 0.1 to about 25 mg/kg body weight. The effective daily amount of the compound of formula 1 generally will be between about 0.0001 to about 10 mg/kg body weight. In some instances the amount of COX-II inhibitor and/or the compound of formula 1 in the combination may be less than would be required on an individual basis to achieve the same desired effect in inhibiting abnormal cell growth.

A compound of formula 1 can also be used with signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, for example, HERCEPTIN™ (Genentech, Inc. of South San Francisco, Calif., USA). Such combinations are useful for treating and preventing abnormal cell growth, including cancer, as described herein.

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein. EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.). These and other EGFR-inhibiting agents can be used in the present invention.

VEGF inhibitors, for example SU-5416 and SU-6668 (Sugen Inc. of South San Francisco, Calif., USA), can also be combined with a compound of formula 1. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97122596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are incorporated herein in their entireties by reference. Other examples of some specific VEGF inhibitors useful in the present invention are IM862 (Cytran Inc. of Kirkland, Wash., USA); anti-VEGF monoclonal antibody of Genentech, Inc. of South San Francisco, Calif.; and angiozyme, a synthetic ribozyme from Ribozyme (Boulder, Colo.) and Chiron (Emeryville, Calif.). These and other VEGF inhibitors can be used in the present invention as described herein.

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), can also be combined with a compound of formula 1, for example those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. 5,587,458 (issued Dec. 24, 1996), and U.S. Pat. No. 5,877,305 (issued Mar. 2, 1999), which are all hereby incorporated herein in their entireties by reference. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are incorporated in their entireties herein by reference. The erbB2 receptor inhibitor compounds and substance described in the aforementioned PCT applications, U.S. patents, and U.S. provisional applications, as well as other compounds and substances that inhibit the erbB2 receptor, can be used with a compound of formula 1, in accordance with the present invention.

A compound of formula 1, can also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as farnesyl protein transferase inhibitors. Specific CTLA4 antibodies that can be used in the present invention include those described in U.S. Provisional Application 60/113,647 (filed Dec. 23, 1998) which is incorporated by reference in its entirety, however other CTLA4 antibodies can be used in the present invention.

The compounds of formula 1 can also be administered in a method for inhibiting abnormal cell growth in a mammal in combination with radiation therapy. Techniques for administering radiation therapy are known in the art, and these techniques can be used in the combination therapy described herein. The administration of the compound of the invention in this combination therapy can be determined as described herein.

Compounds of formula 1 can also be administered in combination with a COX-II inhibitor for treating Alzheimer's disease, mild cognitive impairment, or age-related cognitive decline. Specific examples of COX-II inhibitors useful in this aspect of the invention are provided above, wherein use of a COX-II inhibitor in combination with a compound of formula 1 for treatment of abnormal cell growth is described. The effective amount of a COX-II inhibitor in combination with a compound of formula 1 can generally be determined by a person of ordinary skill. A proposed effective daily dose range for a COX-II inhibitor in combination with a compound of formula 1 is from about 0.1 to about 25 mg/kg body weight. The daily effective amount of the compound of formula 1 generally will be between about 0.0001 to about 10 mg/kg body weight. In some instances the amount of COX-II inhibitor and/or the amount of the compound of formula 1 in the combination may be less than would be required on an individual basis to achieve the same desired effect in treating Alzheimer's disease, mild cognitive impairment, or age-related cognitive decline.

Compounds of formula 1 can also be administered in combination with an NK-1 receptor antagonist for treatment of depression or anxiety. An NK-1 receptor antagonist, as recited herein, is a substance that is able to antagonize NK-1 receptors, thereby inhibiting tachykinin-mediated responses, such as responses mediated by substance P. Various NK-1 receptor antagonists are known in the art, and any such NK-1 receptor antagonist can be utilized in the present invention as described above in combination with a compound of formula 1. NK-1 receptor antagonists are described in, for example, U.S. Pat. No. 5,716,965 (issued Feb. 10, 1998); U.S. Pat. No. 5,852,038 (issued Dec. 22, 1998); WO 90/05729 (international Publication Date May 31, 1990); U.S. Pat. No. 5,807,867 (issued Sep. 15, 1998); U.S. Pat. No. 5,886,009 (issued Mar. 23, 1999); U.S. Pat. No. 5,939,433 (issued Aug. 17, 1999); U.S. Pat. No. 5,773,450 (issued Jun. 30, 1998); U.S. Pat. No. 5,744,480 (issued Apr. 28, 1998); U.S. Pat. No. 5,232,929 (issued Aug. 3, 1993); U.S. Pat. No. 5,332,817 (issued Jul. 26, 1994); U.S. Pat. No. 5,122,525 (issued Jun. 16, 1992), U.S. Pat. No. 5,843,966 (issued Dec. 1, 1998); U.S. Pat. No. 5,703,240 (issued Dec. 30, 1997); U.S. Pat. No. 5,719,147 (issued Feb. 17, 1998); and U.S. Pat. No. 5,637,699 (issued Jun. 10, 1997). Each of the foregoing U.S. patents and the foregoing published PCT International Application are incorporated in their entireties herein by reference. The compounds described in said references having NK-1 receptor antagonizing activity can be used in the present invention. However, other NK-1 receptor antagonists can also be used in this invention.

The effective amount of an NK-1 receptor antagonist in combination with a compound of formula 1 can generally be determined by a person of ordinary skill. A proposed effective daily dose range for an NK-1 receptor antagonist in combination with a compound of formula 1 is from about 0.07 to about 21 mg/kg body weight. The effective amount of the compound of formula 1 will be between about 0.0001 to about 10 mg/kg body weight. In some instances the amount of NK-1 receptor antagonist and/or the amount of the compound of formula 1 in the combination may be less than would be required on an individual basis to achieve the same desired effect in treating depression or anxiety.

The subject invention also provides combining a compound of formula 1 with a 5HT_{1D} receptor antagonist for treatment of depression or anxiety. A 5HT_{1D} receptor antagonist, as recited herein, is a substance that antagonizes the 5HT_{1D} subtype of serotonin receptor. Any such substance can be used in the present invention as described above in combination with a compound of formula 1. Substances having 5HT_{1D} receptor antagonizing activity can be determined by those of ordinary skill in the art. For example, 5HT_{1D} receptor antagonists are described in WO 98/14433 (International Publication Date Apr. 9, 1998); WO 97/36867 (International Publication Date Oct. 9, 1997); WO 94/21619 (International Publication Date Sep. 29, 1994); U.S. Pat. No. 5,510,350 (issued Apr. 23, 1996); U.S. Pat. No. 5,358,948 (issued Oct. 25, 1994); and GB 2276162 A (published Sep. 21, 1994). These 5HT_{1D} receptor antagonists, as well as others, can be used in the present invention. The aforementioned published patent applications and patents are incorporated herein by reference in their entireties.

The effective amount of a 5HT1_{D} receptor antagonist in combination with a compound of formula 1 can generally be determined by a person of ordinary skill. A proposed effective daily dose range for a 5HT1_{D} receptor antagonist in combination with a compound of formula 1 is from about 0.01 to about 40 mg/kg body weight. The effective daily amount of the compound of formula 1 generally will be between about 0.0001 to about 10 mg/kg body weight. In some instances the amount of 5HT1_{D} receptor antagonist and/or the amount of compound of formula 1 in the combination may be less than would be required on an individual basis to achieve the same desired effect in treating depression or anxiety.

This invention also provides a pharmaceutical composition and method for treating depression or anxiety in a mammal comprising a compound of formula 1 and a SSRI. Examples of SSRIs that can be combined in a method or pharmaceutical composition with compounds of formula 1 and their pharmaceutically acceptable salts include, but are not limited to, fluoxetine, paroxetine, sertraline, and fluvoxamine. Other SSRIs may be combined or administered in combination with a compound of formula 1 or a pharmaceutically acceptable salt thereof. Other antidepressants and/or anxiolytic agents with which a compound of formula 1 may be combined or administered include WELLBUTRIN, SERZONE and EFFEXOR.

The effective amount of a SSRI in combination with a compound of formula 1 can generally be determined by a person of ordinary skill. A proposed effective daily dose range for a SSRI in combination with a compound of formula 1 is from about 0.01 to about 500 mg/kg body weight. The effective daily amount of the compound of formula 1 generally will be between about 0.0001 to about 10 mg/kg body weight. In some instances the amount of SSRI and/or the amount of the compound of formula 1 in the combination may be less than would be required on an individual basis to achieve the same desired effect in treating depression or anxiety.

A compound of formula 1, or a pharmaceutically acceptable salt thereof, can also be combined with one or more antipsychotic agents, for example a dopaminergic agent, for the treatment of diseases or conditions the treatment of which can be effected or facilitated by altering dopamine neurotransmission, such as schizophrenia. Examples of antipsychotics with which a compound of the invention can be combined include ziprasidone (5-(2-(4-(1,2-benzisothiazol-3-yl)-1-piperazinyl)ethyl)-6-chloro-1,3-dihydro-2H-indol-2-one; U.S. Pat. No. 4,831,031 and U.S. Pat. No. 5,312,925); olanzapine (2-methyl-4-(4-methyl-1-piperazinyl-10H-thieno (2,3b) (1,5)benzodiazepine; U.S. Pat. No. 4,115,574 and U.S. Pat. No. 5,229,382); risperidone (3-[2-[4-(6-fluoro-1,2-benzisoxazol-3-yl)-1-piperidinyl]ethyl]6,7,8,9-tetrahydro-2-methyl-4H-pyrido[1,2-a]pyrimidin-4-one; U.S. Pat. No. 4,804,663); L-745870 (3-(4-(4-chlorophenyl)piperazin-1-yl)methyl-1H-pyrrolo(2,3-b)pyridine; U.S. Pat. No. 5,432,177); sonepiprazole (S-4-(4-(2-(isochroman-1-yl)ethyl)piperazin-1-yl)benzenesulfonamide; U.S. Pat. No. 5,877,317); RP 62203 (fananserin; 2-(3-(4-(4-fluorophenyl)-1-piperazinyl)propyl)naphtho(1,8-c,d)isothiazole-1,1-dioxide; U.S. Pat. No. 5,021,420); NGD 941 (U.S. Pat. No 5,633,376 and U.S. Pat. No. 5,428,165); balaperidone ((1α,5α,6α)-3-(2-(6-(4-fluorophenyl)-3-azabicyclo(3.2.0)hept-3-yl)ethyl)-2,4(1H,3H)-quinazolinedione; U.S. Pat. No. 5,475,105); flesinoxan ((+)-4-fluoro-N-[2-[4-5-(2-hydroxymethyl-1,4-benzodioxanyl)]-1-piperazinyl]ethyl]benzamide; U.S. Pat. No. 4,833,142); and gepirone (4,4-dimethyl-1-(4-(4-(2-pyrimidinyl)-1-piperazinyl)butyl)-2,6-piperidinedione; U.S. Pat. No. 4,423,049). The patents recited above in this paragraph are each incorporated herein by reference in their entireties. The effective daily amount of the compound of formula 1 will typically be between about 0.0001 to about 10 mg/kg body weight. The amount of any of the aforementioned antipsychotic agents contemplated for use in combination with a compound of formula 1 is generally the amount known in the art to be useful for treating psychotic conditions. However, in some instances, the amount of the antipsychotic and/or the amount of the compound of formula 1 in the combination may be less than would be required on an individual basis to achieve the same desired effect in treating depression or anxiety. It is furthermore to be understood that the present invention also encompasses combining a compound of formula 1 with antipsychotic or dopaminergic other than those in the aforementioned list.

A proposed amount for sonepiprazole in the above-described combination with a compound of formula 1, is from about 0.005 to about 50 mg/kg body weight of the patient per day. A proposed amount of RP 62203 in such combination is from about 0.20 to about 6 mg/kg body weight of the patient per day. A proposed amount of NGD 941 in such combination is from about 0.1 to about 140 mg/kg of body weight per day. A proposed amount of balaperidone in such combination is from about 1 to about 100 mg/kg body weight per day. A proposed amount of flesinoxan in such combination is from about 0.02 to about 1.6 mg/kg body weight per day. A proposed amount for gepirone in such combination is from about 0.01 to about 2 mg/kg body weight per day. A proposed amount of L-745870 in such combination is from about 0.01 to about 250 mg/kg body weight per day, preferably from about 0.05 to about 100 mg/kg body weight per day. A proposed amount of risperidone in such combination is from about 0.05 to about 50 mg/kg body weight per day. A proposed amount of olanzapine in such combination is from about 0.0005 to about 0.6 mg/kg body weight per day. A proposed amount of ziprasidone in such combination is from about 0.05 to about 10 mg/kg body weight per day. In some instances for each of the aforementioned combinations, however, the amount of each specific ingredient in the combination may be less than would be required on an individual basis to achieve the same desired effect in treating a psychotic condition.

This invention also provides a pharmaceutical composition and method for treating Alzheimer's disease, mild cognitive impairment, or age-related cognitive decline comprising a compound of formula 1 and an acetylcholinesterase inhibitor. Acetylcholinesterase inhibitors are known in the art, and any such acetylcholinesterase inhibitor can be used in the above-described pharmaceutical composition or method. Examples of acetylcholinesterase inhibitors that can be used in this invention are ARICEPT (donepezil; U.S. Pat. No. 4,895,841); EXELON (rivastigmine ((S)-[N-ethyl-3-[1-(dimethylamino)ethyl]phenyl carbamate); U.S. Pat. Nos. 5,603,176 and 4,948,807); metrifonate ((2,2,2-trichloro-1-hydroxyethyl)phosphonic acid dimethyl ester; U.S. Pat. Nos. 2,701,225 and 4,950,658); galantamine (U.S. Pat. No. 4,663,318); physostigmine (Forest, USA); tacrine (1,2,3,4-tetrahydro-9-acridinamine; U.S. Pat. No. 4,816,456); huperzine A (5R-(5α,9β,11E))-5-amino-11-ethylidene-5,6,9,10-tetrahydro-7-methyl-5,9-methaneocycloocta(b)pyridin-2-(1H )-one); and icopezil (5,7-dihydro-3-(2-(1-(phenyl methyl)-4-piperidinyl)ethyl)-6H-pyrrolo(3,2-f)-1,2-benzisoxazol-6-one; U.S. Pat. No. 5,750,542 and WO 92/17475). The patents and patent applications recited above in this paragraph are herein incorporated by reference in their entireties.

The effective amount of an acetylcholinesterase inhibitor in combination with a compound of formula 1 can generally be determined by a person of ordinary skill. A proposed effective daily dose range for an acetylcholinesterase inhibitor in combination with a compound of formula 1 is from about 0.01 to about 10 mg/kg body weight. The effective daily amount of the compound of formula 1 generally will be between about 0.0001 to about 10 mg/kg body weight. In some instances the amount of acetylcholinesterase inhibitor and/or the amount of the compound of formula 1 in the combination may be less than would be required on an individual basis to achieve the same desired effect in treating Alzheimer's disease, mild cognitive impairment, or age-related cognitive decline.

The present invention also provides for combining a compound of formula 1 with neuroprotectants, for example NMDA receptor antagonists, for treatment of Huntington's disease, stroke, spinal cord trauma, traumatic brain injury, multiinfarct dementia, epilepsy, amyotrophic lateral sclerosis, pain, viral induced dementia for example AIDS induced dementia, migraine, hypoglycemia, urinary incontinece, brain ischemia, multiple sclerosis, Alzheimer's disease, senile dementia of the Alzheimer's type, mild cognitive impairment, age-related cognitive decline, emesis, corticobasal degeneration, dementia pugilistica, Down's syndrome, myotonic dystrophy, Niemann-Pick disease, Pick's disease, prion disease with tangles, progessive supranuclear palsy, lower lateral sclerosis, or subacute sclerosing panencephalistis. Examples of NMDA receptor antagonists that can be used in the present invention include (1S,2S)-1-(4-hydroxyphenyl)-2-(4-hydroxy-4-phenylpiperidin-1-yl)-1-propanol (U.S. Pat. No. 5,272,160), eliprodil (U.S. Pat. No. 4,690,931), and gavestenel (U.S. Pat. No. 5,373,018). Other NMDA receptor antagonists, which can also be used in the present invention, are described in U.S. Pat. Nos. 5,373,018; 4,690,931; 5,272,160; 5,185,343; 5,356,905; 5,744,483; WO 97/23216; WO 97/23215; WO 97/23214; WO 96/37222; WO 96/06081; WO 97/23458; WO 97/32581; WO 98/18793; WO 97/23202; and U.S. Ser. No. 08/292,651 (filed Aug. 18, 1994). The aforementioned patents and patent applications are each hereby incorporated by reference in their entireties.

The effective daily amount of the compound of formula 1 in the combination with an NMDA receptor antagonist generally will be between about 0.0001 to about 10 mg/kg body weight. The amount of the NMDA receptor antagonist contemplated for use in combination with a compound of formula 1 for treatment of any of the aforementioned disorders, for example Alzheimer's disease, is generally within the range of from about 0.02 mg/kg/day to about 10 mg/kg/day. However, in some instances, the amount of the NMDA antagonist and/or the amount of the compound of formula 1 in the combination may be less than would be required on an individual basis to achieve the same desired effect in treating said disorders.

The subject invention also provides for combining a compound of formula 1 with certain substances capable of treating a stroke or traumatic brain injury, such as TPA, NIF, or potassium channel modulators, for example BMS-204352. Such combinations are useful for treating neurodegenerative disorders such as stroke, spinal cord trauma, traumatic brain injury, multiinfarct dementia, epilepsy, pain, Alzheimer's disease, and senile dementia, for example.

For the above-described combination therapies and pharmaceutical compositions, the effective amounts of the compound of the invention and of the other agent can generally be determined by those of ordinary skill in the art, based on the effective amounts for the compounds described herein and those known or described for the other agent known in the art, for example the amounts described in the above-recited patents and patent application incorporated herein. The formulations and routes of administration for such therapies and compositions can be based on the information described herein for compositions and therapies comprising a compound of the invention as the sole active agent and on information provided for the other agent in combination therewith.

A specific compound of formula 1 can be determined to inhibit cdk2, cdk5, or GSK-3 using biological assays known to those of ordinary skill in the art, for example the assays described below.

The specific activity of a compound of formula 1 for inhibition of cdk5 or cdk2 can, for example, be ascertained by means of the following assays using materials available to those of ordinary skill in the art:

Enzyme activities can be assayed as the incorporation of [33P] from the gamma phosphate of [33P]ATP (Amersham, cat. no. AH-9968) into biotinylated peptide substrate PKTPKKAKKL. In such an assay, reactions are carried out in a buffer containing 50 mM Tris-HCl, pH 8.0; 10 mM MgCl2, 0.1 mM Na3VO4, and 1 mM DTT. The final concentration of ATP is about 0.5 uM (final specific radioactivity of 4uCi/nmol), and the final concentration of substrate 0.75 uM. Reactions, initiated by the addition of either cdk5 and activator protein p25 or cdk2 and activator cyclin E, may be carried out at room temperature for about 60 minutes. Reactions are stopped by addition of 0.6 volume of buffer containing (final concentrations): 2.5 mM EDTA, 0.05% Triton-X 100, 100 uM ATP, and 1.25 mg/ml streptavidin coated SPA beads (Amersham cat. no. RPNQ0007). Radioactivity associated with the beads is quantified by scintillation counting.

The specific activity of a compound of formula 1 for inhibition of GSK-3 can be determined in both cell-fee and cell-based assays, both of which are described in the art (see, for example, WO 99/65897). A cell-free assay can be carried out in general by incubating GSK-3 with a peptide substrate, radiolabeled ATP (such as, for example, γ³³P- or γ³²-P-ATP, both available from Amersham, Arlington Heights, Ill.), magnesium ions, and the compound to be assayed. The mixture is incubated for a period of time to allow incorporation of radiolabeld phosphate into the peptide substrate by GSK-3 activity. The reaction mixture is washed to remove unreacted radiolabeled ATP, typically after first transferring all or a portion of the enzyme reaction mixture to a well that contains a uniform amount of a ligand that is capable of binding to the peptide substrate. The amount of ³³P or ³²P remaining in each well after washing is then quantified to determine the amount of radiolabeled phosphate incorporated into the peptide substrate. Inhibition is observed as a reduction, relative to a control, in the incorporation of radiolabeled phosphate into the peptide substrate. An example of a suitable GSK-3 peptide substrate for an assay is the SGSG-linked CREB peptide sequence, derived from the CREB DNA binding protein, described in Wang, et al., Anal. Biochem., 220:397402 (1994). Purified GSK-3 for an assay may, for example, be obtained from cells transfected with a human GSK-3β expression plasmid as described in, for example Stambolic, et al., Current Biology 6:1664-68 (1996). WO 99/65897; Wang, et al., and Stambolic, et al. are incorporated in their entireties herein by reference.

Another example of a GSK-3 assay, similar to the one described in the preceding paragraph is as follows: Enzyme activities are assayed as the incorporation of [33P] from gamma phosphate of [33P]ATP (Amersham, cat. No. AH-9968) into biotinylated peptide substrate PKTPKKAKKL. Reactions are carried out in a buffer containing 50 mM Tris-HCl, pH 8.0; 10 mM MgCl₂, 0.1 mM Na₃VO₄, and 1 mM DTT. The final concentration of ATP is 0.5 μM (final specific radioactivity of 4 μCi/nmol), and the final concentration of substrate is 0.75 μM. Reactions, initiated by the addition of enzyme, are carried out at room temperature for about 60 minutes. Reactions are stopped by addition of 0.6 volume of buffer containing (final concentrations): 2.5 mM EDTA, 0.05% Triton-X 100, 100 μM ATP, and 1.25 mg/ml streptavidin coated SPA beads (Amersham cat. No. RPNQ0007). Radioactivity associated with the beads is quantified by scintillation counting.

All of the title compounds of the following Examples had an IC₅₀ inhibiting peptide substrate phosphorylation of less than about 50 μM when assayed for cdk5 inhibition according to the preceding assay.

Several of the title compounds of the following Examples were assayed for GSK-3 inhibition using an assay such as that described above, and all tested had an IC₅₀ for inhibition of GSK-3β of less than about 50 μM.

The following Examples illustrate the present invention. It is to be understood, however, that the invention, as fully described herein and as recited in the claims, is not intended to be limited by the details of the following Examples.

In the following Examples “TFA” indicates “trifluoroacetic acid”, “THF” indicates “tetrahydrofuran”, “MPLC” indicates “medium pressure liquid chromatography”, “TLC” indicates “thin layer chromatography”, “KOBu¹” indicates “potassium-t-butoxide”, “DMSO” is “dimethyl sulfoxide”, and “EtOAc” is “ethyl acetate”. “MS”, as in, for example, “Powdered 4 Å MS” is “molecular sieve”.

### EXAMPLES

### Example 1

### (5-Cyclobutyl-1H-pyrazol-3-yl)-(4-nitro-phenyl)-amine

Step 1. 3-Cyclobutyl-N-(4-nitro-phenyl)-3-oxo-thiopropionamide

To a stirring solution of lithium bis(trimethylsilyl)amide (3.6 mL, 3.6 mmol, 1.0 M in tetrahydrofuran) in tetrahydrofuran at −78° C. (acetone/CO₂ bath) was added a solution of methyl cyclobutyl ketone (400 uL, 360 mg, 3.6 mmol) in 10 mL of tetrahydrofuran. After 1 hr 4-nitro-phenyl isothiocyanate (328 mg, 1.8 mmol) was added in one portion. The reactant was allowed to slowly warm to rt overnight. After 16 hr the reaction was quenched with NH₄Cl, diluted with CH₂Cl₂. The layers were separated and the aqueous layer was extracted with CH₂Cl₂, dried over MgSO₄ and concentrated under reduced pressure. Purification of this material was accomplished by MPLC using the Biotage Flash system eluting with a gradient of hexanes through 50% EtOAc/hexanes. The product-containing fractions were collected and concentrated to give 3-cyclobutyl-N-(4-nitro-phenyl)-3-oxo-thiopropionamide (266 mg, 53% yield) as a yellow oil. R_{f}=0.54 (30% acetone/hexanes); ¹H NMR (400 MHz, CDCl₃) δ 2.35-1.80 (m, 6H), 3.41 (dddd, J=8.2, 8.2, 7.9, 7.9 Hz, 1H), 4.04 (s, 2H), 7.70 (d, 8.7 Hz, 1H), 8.11 (d, 9.1 Hz. 2H), 8.23 (d, 9.2 Hz, 2H); LRMS m/z (APCl⁺) 279 (M+1).

Step 2

To a stirring solution of 3-Cyclobutyl-N-(4-nitro-phenyl)-3-oxo-thiopropionamide from Step 1 (266 mg, 0.95 mmol) in 2 mL of EtOH was added 150 uL of acetic acid follow by anhydrous hydrazine (283 uL, 306 mg, 9.6 mmol). The reaction was heated to 71° C. for 2 hr and then cooled to rt. The reaction was then quenched with aqueous NaHCO₃ diluted with EtOAc and then the layers were separated. The organic layer was washed with water and the aqueous layer was back extracted with CH₂Cl₂. The combined organic layers were dried over MgSO₄, filtered and concentrated under reduced pressure. Purification of this material was accomplished by MP LC using the Biotage Flash system eluting with a gradient of hexanes through 50% acetone/hexanes. The product containing fractions were collected and concentrated to give (5-cyclobutyl-1H-pyrazol-3-yl)-(4-nitro-phenyl)-amine (216 mg, 88%) as a tan solid. R_{f} 0.36 (30% acetone/hexanes); ¹H NMR (400 MHz, CDCl₃) δ 1.84-2.22 (m, 4H), 2.38-2.42 (m, 2H), 3,50 (dddd, J=8.7, 8.7, 8.2, 8.2 Hz, 1H), 5.91 (s, 1H), 7.16 (d, J=7.1 Hz, 2H), 8.12 (d, J=7.1 Hz, 2H). LRMS m/z (APCl⁺) 259.3 (M+1).

### Example 2

### (5-Cyclobutyl-2H-pyrazol-3-yl)-naphthalen-2-yl-amine

The title compound was prepared according to the method for Example 1, using analogous reactants.

¹HNMR (400 MHz, CDCl₃), δ 1.75-1.98 (m, 2H), 2.04-2.19 (m, 2H), 2.20-2.28 (m, 2H), 3.26 (dddd, J=8.7, 8.7, 8.3, 8.3 Hz, 1H), 5.96 (s, 1H), 7.18 (dd, J=9.1, 8.7 Hz, 1H), 7.31 (d, J=7.8 Hz, 1 H), 7.37 (d, J=7.9 Hz, 1H), 7.53 (s, 1H), 7.61-7.71 (m, 3H). LRMS m/z (APCl⁺) 264 (M+1).

### Example 3

### (5-Cyclobutyl-2H-pyrazol-3-yl)-naphthalen-1-yl-amine

The title compound was prepared according to the method for Example 1, using analogous reactants.

R_{f} 0.35 (50% EtOAc/Hexane), ¹H NMR (400 MHz, CDCl₃) δ 1.75-1.98 (m, 2H), 2.04-2.19 (m, 2H), 2.20-2.28 (m, 2H), 3.36-3.44 (dddd, J=8.7, 8.7, 8.7, 8.7 Hz, 1H), 5.92 (s, 1H), 7.31-7.44 (m, 5H), 7.80 (d, J=8.31 Hz, 1H), 8.98 (d, J=8.72 Hz, 1H). LRMS m/z (APCl⁺) 264 (M+1).

### Example 4

### N-(5-Cyclobutyl-2H-pyrazol-3-yl)-N′,N′-dimethyl-naphthalene-1,4-diamine

The title compound was prepared according to the method for Example 1, using analogous reactants.

¹HNMR (400 MHz, CDCl₃), δ 1.73-1.90 (m, 2H), 2.09-2.28 (m, 4H), 2.85 (s, 6H), 3.37 (dddd, J=8.7, 8.7, 8.3, 8.3 Hz, 1 H), 5.81 (s, 1H), 7.02 (d, J=8.3 Hz, 1H), 7.36 (d, J=7.8 Hz, 1H), 7.40 (dd, J=6.6, 6.6 Hz, 1H), 7.51 (dd, J=4.8, 4.8 Hz, 1H), 8.04 (d, J=8.3 Hz, 1H), 8.30 (d, J=8.3 Hz, 1H). ¹³C NMR (100 MHz, CDCl₃), δ 153.4, 150.2, 145.5, 135.1, 129.9, 127.7, 128.6, 124.7, 122.1, 114.7, 113.4, 91.5, 45.8, 32.3, 29.5, 18.9. LRMS m/z (APCl⁺) 307.3 (M+1).

### Example 5

### (3-Benzyloxy-phenyl)-(5-cyclobutyl-2H-pyrazol-3-yl)-amine

The title compound was prepared according to the method for Example 1, using analogous reactants. ¹HNMR (400MHz, CDCl₃), δ 1.82-2.00 (m, 2H), 2.09-2.20 (m, 2H), 2.24-2.32 (m, 2H), 3.41 (dddd, J=8.7, 8.7, 8.3, 8.3 Hz, 1H), 5.01 (s, 2H), 5.88 (s, 1H), 6.47 (bs, 1H), 6.50 (dd, J=7.5, 1.6 Hz, 1H), 6.67 (dd, J=9.5, 1.6 Hz, 1H), 6.81 (dd, J=2.0, 2.0 Hz, 1H), 7.14 (dd, J=7.9, 8.3 Hz, 1H), 7.31-7.43 (m, 5H). LRMS m/z (APCl⁺) 320.4 (M+1).

### Example 6

### (4-Chloro-benzyl)-(5-cyclobutyl-2H-pyrazol-3-yl)-amine

The title compound was prepared according to the method for Example 1, using analogous reactants.

¹HNMR (400 MHz, CDCl₃), δ 1.85-2.15 (m, 4H), 2.26-2.32 (m, 2H), 3.39 (dddd, J=8.7, 8.7, 8.3, 8.3 Hz, 1H), 4.29 (s, 2H), 5.41 (s, 1H), 5.80 (bs, 2H), 7.26 (7.31 (m, 4H), LRMS m/z (APCl⁺) 262.3 (M+1).

### Example 7

### (3-Bromo-phenyl)-(5-cyclobutyl-2H-pyrazol-3-yl)-amine

The title compound was prepared according to the method for Example 1, using analogous reactants.

¹HNMR (400 MHz, CDCl₃), δ 1.82-1.99 (m, 2H), 2.06-2.17 (m, 2H), 2.21-2.29 (m, 2H), 3.39 (dddd, J=8.8, 8.8, 8.8, 8.8 Hz, 1H), 5.86 (s, 1H), 6.78 (bs, 1H), 6.98-6.92 (m, 2H), 7.04 (dd, J=7.9, 7.8 Hz, 1 H), 7.24 (s, 1 H), 9.80 (bs, 1 H). LRMS m/z (APCl⁺) 292.3, 294.2 (M+1).

### Example 8

### [5-(1,4-Dioxa-spiro[4.4]non-7-yl)-1H-pyrazol-3-yl]-(3-trifluoromethyl-phenyl)-amine

The title compound was prepared according to the method for Example 1, using analogous reactants.

¹HNMR (400 MHz, CDCl₃), δ 1.72-2.16 (m, 5H), 2.25 (ddd, J=13.7, 8.7, 0 Hz, 1H), 3.26 (dddd, J=8.3, 8.2, 8.2, 7.8 Hz, 1H), 3.91-3.97 (m, 4H), 5.83 (s, 1H), 6.71 (bs, 1H), 7.06-7.32 (m, 4H), 9.0 (bs, 1H). LRMS m/z (APCl⁺) 354.1 (M+1).

### Example 9

### (2-Chloro-4-nitro-phenyl)-(5-cyclobutyl-2H-pyrazol-3-yl)-amine

The title compound was prepared according to the method for Example 1, using analogous reactants.

R_{f} 0.35 (30% EtOAc/Hexane), ¹H NMR (400 MHz, CDCl₃), δ 1.90-2.23 (m, 4H), 2.36-2.44 (m, 2H), 3.48-3.57 (dddd, J=8.3, 8.3, 8.3, 8.3 Hz, 1H), 5.96 (s, 1H), 7.72-7.76 (d, 1H), 8.05-8.08 (d, 1H), 8.26 (s, 1H). LRMS m/z (APCl⁺) 293 (M+1).

### Example 10

### (3,5-Bis-trifluoromethyl-phenyl)-(5-cyclobutyl-2H-pyrazol-3-yl)-amine

The title compound was prepared according to the method for Example 1, using analogous reactants.

R_{f} 0.35 (30% EtOAc/Hexane), ¹H NMR (400 MHz, CDCl₃), δ 1.80-2.21 (m, 4H), 2.31-2.40 (m, 2H), 3.47-3.51 (dddd, J=8.7, 8.7, 8.7, 8.7 Hz, 1H), 5.83 (s, 1H), 7.26 (s, 1H), 7.56 (s, 1H), 7.56 (s, 1H). LRMS m/z (APCl⁺) 350 (M+1).

### Example 11

### 4-(5-Cyclobutyl-2H-pyrazol-3-ylamino)-benzonitrile

The title compound was prepared according to the method for Example 1, using analogous reactants.

R_{f} 0.38 (50% EtOAc/Hexane), ¹H NMR (400 MHz, CD₃OD), δ 1.88-2.11 (m, 2H), 2.15-2.25 (m, 2H), 2.32-2.40 (m, 2H), 3.49-3.57 (dddd, J=8.3, 8.3, 8.3, 8.3 Hz, 1H), 5.85 (s, 1H), 7.22-7.25 (dd, J=2.5 Hz, 2H), 7.46-7.49 (dd, J=2.5 Hz, 2H). LRMS m/z (APCl⁺) 239 (M+1).

### Example 12

### (5-Cyclobutyl-2H-pyrazol-3-yl)-(3-fluoro-phenyl)-amine

The title compound was prepared according to the method for Example 1, using analogous reactants.

R_{f} 0.30 (50% EtOAc/Hexane), ¹H NMR (500 MHz, CDCl₃), δ 1.86-2.04 (m, 2H), 2.12-2.19 (m, 2H), 2.30-2.36 (m, 2H), 3.43-3.49 (dddd, J=8.8, 8.8, 8.8, 8.8 Hz, 1H), 5.89 (s, 1H), 6.53-6.57 (m, 1H), 6.80-6.82 (m, 1H), 6.91-6.94 (m, 1H), 7.14-7.19 (m, 1H), LRMS m/z (APCl⁺) 232 (M+1).

### Example 13

### (2-Bromo-phenyl)-(5-cyclobutyl-2H-pyrazol-3-yl)-amine

The title compound was prepared according to the method for Example 1, using analogous reactants.

R_{f} 0.33 (30% EtOAc/Hexane). ¹HNMR (400 MHz, CDCl₃), δ 1.90-2.12 (m, 2H), 2.13-2.22 (m, 2H), 2.33-2.41 (m, 2H), 3.44-3.51 (dddd, J=8.3, 8.3, 8.3, 8.3 Hz, 1H), 5.90 (s, 1H), 6.69-6.73 (dd, J=6.6, 6.6 Hz, 1H), 7.18-7.22 (dd, J=5.8, 5.8 Hz, 1H), 7.47-7.49 (d, 1H), 7.598-7.59 (d, 1H). LRMS m/z (APCl⁺) 292 (M+1).

### Example 14

### (5-Cyclobutyl-2H-pyrazol-3-yl)-(3,5-dichloro-phenyl)-amine

The title compound was prepared according to the method for Example 1, using analogous reactants.

R_{f} 0.50 (50% EtOAc/Hexane), ¹H NMR (CDCl₃), 6 1.86-2.07 (m, 2H), 2.09-2.19 (m, 2H), 2.29-2.38 (m, 2H), 3.42-3.50 (dddd, J=8.5, 8.5, 8.5, 8.5 Hz, 1 H), 5.84 (s, 1H), 6.80 (s, 1H), 6.98 (s, 1H), 6.98 (s, 1H). LRMS m/z (APCl⁺) 282 (M+1).

Preparation 1.1. 2-Cyano-3-cyclobutyl-3-oxo-propionic acid ethyl ester

To anhydrous MgCl₂ (22.3 g, 0.19 mmol) in 320 mL of CH₃CN at 0° C. was added ethyl cyanoacetate (21.5 g, 0.19 mmol). After 15 min Et₃N (52.0 mL, 38.0 g, 0.37 mmol) was added via syringe. The reaction was allowed to stir an additional 15 min and then cyclobutane carbonyl chloride (21.0 mL 22.3 g, 0.19 mmol) was added over a five min period. The reaction was allowed to slowly warm to rt over 20 hr. The reaction was then cooled to 0° C., quenched with aqueous 0.5 M HCl, and then diluted with 150 mL of Et₂O. The aqueous layer was extracted with Et₂O (3×150 mL) and then the combined organic layers were washed with 150 mL of brine, dried over MgSO₄, filtered and concentrated to give a yellow-orange oil. This material was purified by vacuum distillation (95-105° C., 2-3 mm) to give the title compound in a quantitative yield and as a colorless oil.

¹H NMR (400MHz, CDCl₃), δ 1.32-1.36 (t, 3H), 1.88-2.09 (m, 2H), 2.21-2.28 (m, 2H), 2.32-2.43 (m, 2H), 3.64-3.69 (dddd, J=8.5, 8.5, 8.5, 8.5 Hz, 1H), 4.28-4.34 (q, 2H), 13.81 (s, 1H).

Preparation 2.1. 2-Cyano-3-oxo-pentanoic acid ethyl ester

The title compound was prepared according to the method for Preparation 1.1, using analogous reactants.

¹H NMR (400 MHz, CDCl₃), δ 1.23-1.27 (t, 3H), 1.34-1.37 (t, 3H), 2.61-2.66 (q, 2H), 4.30-4.35 (q, 2H), 13.70 (s, 1 H). LRMS m/z (APCl⁻) 168 (M−1).

Preparation 1.2. 3-Cyclobutyl-3-oxo-propionitrile

To 2-cyano-3-cyclobutyl-3-oxo-propionic acid ethyl ester (Preparation 1.1) in 40 mL of DMSO was added 2 mL of H₂O and then the reaction was heated to 118° C. After 35 min the reaction was cooled in an ice water bath and then quenched with a saturated solution of NaCl. This reaction mixture was further diluted with H₂O and CH₂Cl₂ and the layers were separated. The aqueous layer was extracted with CH₂Cl₂. The combined organic layers were dried over MgSO₄, filtered and concentrated under reduced pressure to give the title compound of Preparation 1.2 as a yellow oil. This material was used without further purification.

¹H NMR (400 MHz, CDCl₃), δ 1.83-2.17 (m, 2H), 2.19-2.30 (m, 2H), 2.31-2.55 (m, 2H), 3.39 (s, 2H), 3.39-3.43 (dddd, J=8.5, 8.5, 8.5, 8.5 Hz, 1H). LRMS m/z (APCl⁻) 122 (M−1).

Preparation 1.3. 5-Cyclobutyl-2-(4-methoxy-benzyl)-2H-pyrazol-3-ylamine

To crude 3-cyclobutyl-3-oxo-propionitrile prepared above in 515 mL of EtOH was added 4-methoxy-benzyl-hydrazine (12.6 g, 82.9 mmol) and the reaction was heated to reflux (oil bath at 85° C.). After 2 hr the reaction was cooled to rt and concentrated under reduced pressure to give a viscous oil. Purified of this material was accomplished by MPLC using the Biotage Flash 45S system eluting with a gradient of 10% and 20% EtOAc/hexanes. The product containing fractions were collected and concentrated to give the title compound (10.2 g, 77% yield over 2 steps) as a colorless solid.

R_{f} 0.3 (40% EtOAc/Hexane), ¹H NMR (400 MHz, CDCl₃), δ 1.85-2.00 (m, 2H), 2.12-2.20 (m, 2H), 2.25-2.31 (m, 2H), 3.41-3.46 (dddd, J=8.1, 8.1, 8.1, 8.1 Hz, 1H), 3.76 (s, 3H), 5.07 (s, 2H), 5.46 (s, 1H), 6.82-6.84 (d, J=6.64 Hz, 2H), 7.07-7.09 (d, J=6.64 Hz, 2H), LRMS m/z (APCl⁺) 258 (M+1).

Preparation 2.3. 5-Ethyl -2-(4-methoxy-benzyl)-2H-pyrazol-3-yl amine

The title compound was prepared according to the method for Preparation 1.3, using Preparation 2.1 instead of Preparation 1.1.

R_{f} 0.4 (50% EtOAc/Hexane, ¹H NMR (400 MHz, CDCl₃), δ 1.16-1.20 (t, 3H), 2.49-2.55 (q, 2H), 3.74 (s, 3H), 5.05 (s, 2H), 5.36 (s, 1H), 6.80-6.82 (d, 2H), 7.06-7.08 (d, 2H), LRMS m/z (APCl⁺) 232 (M+1).

Preparation 1.4. [5-Cyclobutyl-2-(4-methoxy-benzyl)-2H-pyrazol-3-yl]-(6-methoxy-pyridin-2-yl)-amine

Procedure A: A dry toluene solution of 5-Cyclobutyl-2-(4-methoxy-benzyl)-2H-pyrazol-3-ylamine (Preparation 1.3, 2.27 g, 8.83 mmol), 2-chloro-6-methoxy pyridine (1.06 g, 7.36 mmol), sodium tert-butoxide (1.09 g, 10.3 mmol), 2-(dicyclohexylphosphino)-biphenyl (258 mg, 0.736 mmol), and palladium acetate (165 mg, 0.736 mmol), was heated to 120° C. for an hour and then cooled to rt. The reaction was then filtered through Celite pad and the filtrate was concentrated down and chromatographed using the Biotage Flash 45S system eluting with 20% EtOAc/hexanes to give the title compound of Preparation 1.4 as a peach colored solid (2 g, 74% yield).

R_{f} 0.25 (20% EtOAc/Hexane), ¹H NMR (400 MHz, CDCl₃), δ 1.82-2.00 (m, 2H), 2.11-2.34 (m, 4H), 3.45-3.66 (dddd, J=8.7, 8.7, 8.7, 8.7 Hz, 1H), 3.71 (s, 3H), 3.80 (s, 3H), 5.10 (s, 2H), 6.01-6.03 (d, 1H), 6.07 (s, 1H), 6.14-6.16 (d, 1H), 6.75-6.78 (d, J=6.65 Hz, 2H), 7.03-7.06 (d, J=8.72 Hz, 2H), 7.29-7.33 (apt, 1H). LRMS m/z (APCl⁺) 365 (M+1).

Procedure B. Cesium carbonate (296 mg, 0.454 mmol) was flame dried in a reaction flask and 3.9 ml of dry toluene was then transferred followed by 200 mg (0.778 mmol) of 5-Cyclobutyl-2-(4-methoxy-benzyl)-2H-pyrazol-3-ylamine (Preparation 1.3). The resulting mixture was stirred at rt for 10 min followed by the additions of 2-chloro-6-methoxy pyridine (93.2 mg, 0.648 mmol), 2-(dicyclohexylphosphino)-biphenyl (11.7 mg, 0.0334 mmol) and palladium acetate (3.6 mg, 0.0162 mmol). Refluxing under nitrogen overnight took the reaction to completion. Filtration through Celite, concentration of the filtrate, and chromatography as in procedure A yielded the title compound of Preparation 1.4 (120 mg, 51% yield) as a solid.

Procedure C. To a dry toluene (1.95 ml) solution of 5-Cyclobutyl-2-(4-methoxy-benzyl)-2H-pyrazol-3-ylamine (Preparation 1.3, 100 mg, 0.389 mmol) was added 1.0M potassium t-butoxide (628 μl, 0.628 mmol) at rt. After 5min stirring, 3-bromo-anisole (60 mg, 0.324 mmol), 2-(dicyclohexylphosphino)-biphenyl (23 mg, 0.065 mmol), and palladium acetate (7.5 mg, 0.032 mmol) were added and the reaction was heated to 105° C. for 6 hours when the reaction went to completion. The reaction was cooled to rt, filtered through Celite, and chromatographed as in the procedure A to give the title compound of Preparation 1.4 (72 mg, 61% yield) as a viscous gum.

R_{f} 0.30 (30% EtOAc/hexanes), ¹HNMR (400 MHz, CDCl₃), δ 1.86-2.04 (m, 2H), 2.12-2.19 (m, 2H), 2.30-2.36 (m, 2H), 3.54 (dddd, J=8.8, 8.8, 8.8, 8.8 Hz, 1H), 3.73 (s, 3H), 3.77 (s, 3H), 5.12 (s, 2H), 5.98 (s, 1H), 6.29 (d, J=10.7 Hz, 1H), 6.32-6.35 (m, 1H), 6.40-6.43 (m, 1H), 6.82 (d, J=6.6 Hz, 2H), 7.10 (d, J=8.3 Hz, 2H), 7.25 (s, 1H). LRMS m/z (APCl⁺) 364.2 (M+1).

Preparation 2.4. [5-Ethyl-2-(4-methoxy-benzyl)-2H-pyrazol-3-yl]-(6-methoxy-pyridin-2-yl)-amine

The title compound was prepared according to the method for Preparation 1.4, using Preparation 2.3 instead of Preparation 1.3.

R_{f} 0.28 (25% EtOAc/Hexane), ¹H NMR (400 MHz, CDCl₃), δ 1.22-1.26 (t, 3H), 2.60-2.66 (q, 2H), 3.74 (s, 3H), 3.82 (s, 3H), 5.12 (s, 2H), 6.01-6.02 (d, 1H), 6.04 (s, 1H), 6.16-6.18 (d, 1H), 6.79-6.81 (d, J=6.64 Hz, 2H), 7.07-7.09 (d, J=6.64 Hz, 2H), 7.32-7.36 (apt, 1H). LRMS m/z (APCl⁺) 339 (M+1).

### Example 15

### (5-Cyclobutyl-2H-pyrazol-3-yl)-(3-methoxy-phenyl)-amine

To [5-Cyclobutyl-2-(4-methoxy-benzyl)-2H-pyrazol-3-yl]-(3-methoxy-phenyl)-amine (Preparation 1.4, 120 mg, 0.331 mmol) was added neat trifluoro acetic acid (2.0 mL) and the reaction was heated to 70° C. After 48 hr the reaction was cooled to rt and concentrated under reduced pressure. Purification of this material was accomplished by MPLC using the Biotage Flash system eluting with 40% EtOAc/hexanes. The product-containing fractions were collected and concentrated to give the title compound (30 mg, 37% yield).

R_{f} 0.35 (75% EtOAc/Hexane), ¹H NMR (400 MHz, CDCl₃), δ 1.80-2.03 (m, 2H), 2.12-2.17 (m, 2H), 2.29-2.33 (m, 2H), 3.40-3.48 (dddd, J=8.3, 8.3, 8.3, 8.3 Hz, 1H), 3.77 (s, 3H), 5.88 (s, 1H), 6.41-6.43 (d, J=10.4 Hz, 1H), 6.65-6.68 (m, 1H), 6.71-6.72 (m, 1H), 7.11-7.15 (apt, 1H). LRMS m/z (APCl⁺) 244 (M+1).

### Example 16

### (5-Cyclobutyl-2H-pyrazol-3-yl)-(6-trifluoromethyl-pyridin-2-yl)-amine

The title compound was made according to the method for Example 15, using analogous reactants.

R_{f} 0.50 (10% MeOH/CH₂Cl₂), ¹H NMR (400 MHz, CD₃OD), δ 1.86-2.19 (m, 2H), 2.21-2.32 (m, 2H), 2.32-2.39 (m, 2H), 3.51-3.57 (dddd, J=8.7, 8.7, 8.7, 8.7 Hz, 1H), 6.15 (s, 1H), 7.10 (d, J=9.13 Hz, 1H), 7.73-7.76 (d, J=8.72 Hz, 1H), 8.38 (s, 1H). LRMS m/z (APCl⁺) 283 (M+1).

### Example 17

### (5-Cyclobutyl-2H-pyrazol-3-yl)-(3-trifluoromethyl-phenyl)-amine

The title compound was made according to the method for Example 15, using analogous reactants.

R_{f} 0.30 (40% EtOAc/Hexane), ¹H NMR (400 MHz, CDCl₃), δ 1.92-2.26 (m, 4H), 2.38-2.46 (m, 2H), 3.50-3.58 (dddd, J=8.3, 8.3, 8.3, 8.3 Hz, 1H), 6.01 (s, 1H), 7.31-7.47 (m, 4H), LRMS m/z (APCl⁺) 282 (M+1).

### Example 18

### N-(5-Cyclobutyl-2H-pyrazol-3-yl)-N′,N′-dimethyl-benzene-1,3-diamine

The title compound was made according to the method for Example 15, using analogous reactants.

R_{f} 0.50 (5% MeOH/CH₂Cl₂), ¹H NMR (400 MHz, CDCl₃), δ 1.91-2.18 (m, 2H), 2.20-2.30 (m, 2H), 2.35-2.43 (m, 2H). 3.11 (s, 6H), 3.48-3.56 (dddd, J=8.3, 8.3, 8.3, 8.3 Hz, 1H), 6.13 (bs, 1H), 6.84-6.95 (m, 2H), 7.13-7.28 (m, 2H), 7.31-7.35 (apt, 1H). LRMS m/z (APCl⁺) 257 (M+1).

### Example 19

### (5-Cyclobutyl-2H-pyrazol-3-yl)-quinolin-2-yl-amine

The title compound was made according to the method for Example 15, using analogous reactants.

R_{f} 0.35 (70% EtOAc/Hexane), ¹H NMR (400 MHz, CD₃OD), δ 1.92-2.20 (m, 2H), 2.22-2.28 (m, 2H), 2.41-2.45 (m, 2H), 3.60-3.66 (dddd, J=8.3, 8.3, 8.3, 8.3 Hz, 1H), 6.08 (s, 1H), 7.26-7.28 (d, J=9.54 Hz, 1 H), 7.60-7.64 (m, 1 H), 7.87-7.88 (m, 2H), 7.89-8.00 (d, J=7.46 Hz, 1H), 8.49-8.51 (d, 1H). LRMS m/z (APCl⁺) 265 (M+1).

### Example 20

### (6-Chloro-pyridin-2-yl)-(5-cyclobutyl-2H-pyrazol-3-yl)-amine

The title compound was made according to the method for Example 15, using analogous reactants.

R_{f} 0.40 (75% EtOAc/Hexane), ¹H NMR (400 MHz, CD₃OD), δ 1.98-2.21 (m, 2H), 2.24-2.34 (m, 2H), 2.42-2.49 (m, 2H), 3.62-3.68 (dddd, J=8.8, 8.8, 8.8, 8.8 Hz, 1H), 6.18 (s, 1H), 6.87-6.89 (d, J=8.29 Hz, 1H), 7.05-7.07 (d, J=7.47 Hz, 1H), 7.72-7.76 (apt, 1H), LRMS m/z (APCl⁺) 249 (M+1).

### Example 21

### (5-Cyclobutyl-2H-pyrazol-3-yl)-(6-methoxy-4-methyl-quinolin-2-yl)-amine

The title compound was made according to the method for Example 15, using analogous reactants.

R_{f} 0.35 (75% EtOAc/Hexane), ¹H NMR (400 MHz, CD₃OD), δ 1.90-2.18 (m, 2H), 2.19-2.30 (m, 2H), 2.37-2.47 (m, 2H), 2.78 (s, 3H), 3.60-3.68 (dddd, J=8.7, 8.7, 8.7, 8.7 Hz, 1H), 3.97 (s, 3H), 6.02 (s, 1H), 7.10 (s, 1H), 7.45 (s, 1H), 7.46 (d, 1H), 7.78-7.80 (d, 1H), LRMS m/z (APCl⁺) 309 (M+1).

### Example 22

### (5-Cyclobutyl-2H-pyrazol-3-yl)-(3-trifluoromethoxy-phenyl)-amine

The title compound was made according to the method for Example 15, using analogous reactants.

R_{f} 0.15 (30% EtOAc/Hexane), ¹H NMR (400 MHz, CDCl₃), δ 1.88-2.07 (m, 2H), 2.10-2.20 (m, 2H), 2.30-2.38 (m, 2H), 3.42-3.51 (dddd, J=8.3, 8.3, 8.3, 8.3 Hz, 1H), 5.84 (s, 1H), 6.68-6.70 (d, 1H), 6.99-7.19 (m, 2H), 7.21-7.24 (apt, 1H). LRMS m/z (APCl⁺) 298 (M+1).

### Example 23

### N-(5-Cyclobutyl-2H-pyrazol-3-yl)-N′,N′-dimethyl-pyridine-2,6-diamine

The title compound was made according to the method for Example 15, using analogous reactants.

R_{f} 0.50 (5% MeOH/CH₂Cl₂), ¹H NMR (400 MHz, CDCl₃), δ 1.82-2.01 (m, 2H), 2.11-2.21 (m, 2H), 2.26-2.33 (m, 2H), 3.06 (s, 6H), 3.42-3.48 (dddd, J=8.7, 8.7, 8.7, 8.7 Hz, 1H). 5.83 (s, 1H), 5.93 (d, J=8.31 Hz, 1H), 6.09 (d, J=7.89 Hz, 1H), 7.29-7.33 (1H, apt). LRMS m/z (APCl⁺) 258 (M+1).

### Example 24

### (5-Ethyl-2H-pyrazol-3-yl)-(6-methoxy-pyridin-2-yl)-amine

The title compound was made according to the method for Example 15, using analogous reactants.

R_{f} 0.20 (50% EtOAc/Hexane), ¹H NMR (400 MHz, CDCl₃), δ 1.20-1.28 (m, 3H), 2.60-2.68 (m, 2H), 3.90-(s, 3H), 6.25-6.27 (d, 1H), 6.45-6.47 (d, 1H), 6.54 (s, 1H), 7.42-7.46 (apt. 1H). LRMS m/z (APCl⁺) 219 (M+1).

### Example 25

### (5-Cyclobutyl-2H-pyrazol-3-yl)-(6-methoxy-pyridin-2-yl)-amine

The title compound was made according to the method for Example 15, using analogous reactants.

R_{f} 0.30 (50% EtOAc/Hexane), ¹H NMR (400 MHz, CDCl₃), δ 1.87-2.23 (m, 4H), 2.33-2.41 (m, 2H), 3.46-3.55 (dddd, J=8.3, 8.3, 8.3, 8.3 Hz, 1H), 3.93 (s, 3H), 6.26-6.28 (d, 1H), 6.46-6.47 (d, 1H), 6.57 (s, 1H), 7.43-7.47 (apt, 1H). LRMS m/z (APCl⁺) 245 (M+1).

Preparation 3.1. 1,4-Dioxa-spiro[4.4]non-7-yl)-ethanone

To a stirring solution of known nitrile (1.25 g, 8.17 g)((Aust. J. Chem. 1994, 47, 1833) in tetrahydrofuran at rt was added a solution of methyl magnesium bromide (5.4 mL, 16.3 mmol, 3.0M in THF) followed by copper (I) bromide (23 mg, 0,16 mmol). The reaction was then heated to 65° C. After 20 hr the reaction cooled to rt and then to 0° C. (ice/water bath) and quenched with a saturated solution of NH₄Cl. This mixture was diluted with methylene chloride (100 mL) and the layers separated. The aqueous layer was extracted with methylene chloride (100 mL) and the combined organic layers were dried over MgSO₄, filtered and concentrated under reduced pressure. Purification was accomplished by vacuum distillation (120° C., 2 mm) to give the title compound (1.39 g, 51% yield) as a clear colorless oil.

¹HNMR (400 MHz, CDCl₃), δ 1.79-2.08 (m, 6H), 2.15 (s, 3H), 2.99 (dddd, J=8.3 Hz, 1H), 3.87-3.94 (m, 4H). LRMS m/z (APCl⁺) 171 (M+1).

Preparation 3.2. 3-(1,4-Dioxa-spiro[4.4]non-7-yl)-3-oxo-N-(3-trifluoromethyl-phenyl)-thiopropionamide

To a stirring solution of LiHMDS(3.2 mL, 3.2 mmol 1 M in THF) in 20 mL of THF at −78° C. (acetone/CO₂) was a precooled solution (−78° C.) of 1-(1,4-Dioxa-spiro[4.4]non-7-yl)-ethanone (Preparation 3.1, 500 mg, 2.94 mmol in 5 mL of THF) via cannula down the side of the flask. After 30 minutes, 1-isothiocyanato-3-trifluoromethyl-benzene (988 uL, 1.3 g, 6.5 mmol) was added dropwise via syringe. The resulting reaction was slowly allowed to warm to rt overnight. The reaction was then quenched with NaHCO₃, diluted with EtOAc and the layers were separated. Purification of this material was accomplished by MPLC using the Biotage Flash 45S system eluting with 20% EtOAc/toluene, followed by a second MPLC using the Biotage Flash 45S system eluting with 20% acetone/hexanes. The product-containing fractions were collected and concentrated to give the title compound (598 mg, 55% yield) as a yellow viscous gum.

R_{f} 0.3 (25% Acetone/Hexanes). ¹HNMR (400 MHz, CDCl₃), δ 1.84-1.87 (m, 2H), 1.95-2.20 (m, 2H), 2.64-2.70 (m, 1H), 3.28-3.32 (m, 1H), 3.89-4.05 (m, 4H), 3.92 (s, 2H), 7.50-7.54 (m, 2H), 7.93-7.97 (m, 1H), 8.13-8.15 (m, 1H). LRMS m/z (APCl⁺) 374 (M+1).

Preparation 3.3. [5-(1,4-Dioxa-spiro[4.4]non-7-yl)-2-(4-methoxy-benzyl)-2H-pyrazol-3-yl]-(3-trifluoromethyl-phenyl)-amine

To a stirring solution of 3-(1,4-Dioxa-spiro[4.4]non-7-yl)-3-oxo-N-(3-trifluoromethyl-phenyl)-thiopropionamide (Preparation 3.1, 785 mg, 2.1 mmol) in 21 mL of EtOH was added acetic acid (2.1 mL) followed by 4-methoxy-benzyl-hydrazine (480 mg, 3.2 mmol) and the resulting mixture was heated to 75° C. After 1 hr the reaction was cooled to rt quenched with H₂O, diluted with EtOAc and the layers were separated. To the aqueous layer was added a few drops of concentrated ammonium hydroxide and then extracted with EtOAc. The combined organic layers were dried over MgSO₄, filtered and concentrated under reduced pressure. Purification of this material was accomplished by MPLC using the Biotage Flash 45S system eluting with 40% EtOAc/hexanes. The product-containing fractions were collected and concentrated under reduced pressure to give the title compound (788 mg, 79% yield) as a yellow viscous gum.

R_{f} 0.3 (50% EtOAc/Hexanes). ¹HNMR (400 MHz, CDCl₃), δ 1.78-2.08 (m, 4H), 2.16-2.20 (m, 1H), 2.31-2.38 (m, 1H), 3.30 (dddd, J=7.9, 9.5, 8.3, 8.7 Hz, 1H), 3.77 (s, 3H), 3.85-4.00 (m, 4H), 5.14 (s, 2H), 5.95 (s, 1H), 6.81-6.90 (m, 3H), 7.07-7.11 (m, 3H), 7.26-7.29 (m, 2H). LRMS m/z (APCl⁺) 474 (M+1).

Preparation 3.4. 3-[1-(4-Methoxy-benzyl)-5-(3-trifluoromethyl-phenylamino)-1H-pyrazol-3-yl]-cyclopentanone

To a stirring solution of [5-(1,4-Dioxa-spiro[4.4]non-7-yl)-2-(4-methoxy-benzyl)-2H-pyrazol-3-yl]-(3-trifluoromethyl-phenyl)-amine (Preparation 3.3, 679 mg, 1.4 mmol) in 14 mL of acetone was added 700 uL H₂O followed by a catalytic amount of p-toluenesulfonic acid monohydrate (27.3 mg, 0.14 mmol). The reaction mixture was then heated to 65° C. After 1 hr the reaction was cooled to rt and quenched with H₂O, diluted with EtOAc and the layers were separated. The organic layer was dried over MgSO₄, filtered and concentrated under reduced pressure to give the title compound (617 mg, quantitative yield) as a yellow viscous oil. This material was used without further purification.

R_{f} 0.28 (50% EtOAc/Hexanes). ¹HNMR (400 MHz, CDCl₃), δ 2.02-2.14 (m, 1H), 2.20-2.30 (m, 1H), 2.36-2.50 (m, 3H), 2.58-2.63 (m, 1H), 3.45 (dddd, J=6.2, 6.2, 6.2, 6.2 Hz, 1H), 3.76 (s, 3H), 5.11 (s, 2H), 5.94 (s, 1H), 6.79-6.88 (m, 4H), 7.04-7.10 (m, 3H), 7.25-7.29 (m, 1H). LRMS m/z (APCl⁺) 430 (M+1).

### Example 26

### 3-Trans-[5-(3-trifluoromethyl-phenylamino)-2H-pyrazol-3-yl]-cyclopentanone

The title compound was prepared according to the method for Preparation 3.4, using hydrazine instead of 4-methoxy-benzyl-hydrazine (see synthesis of Preparation 3.3, supra).

¹HNMR (400 MHz, CDCl₃), δ 1.94-1.99 (m, 1H), 2.20-2.48 (m, 4H). 2.62 (ddd, J=18.2, 7.9, 0 Hz, 1H), 3.38 (dddd, J=9.5, 9.5, 7.5, 6.6 Hz, 1H), 5.85 (s, 1H), 7.09 (d, J=7.4 Hz, 1H), 7.20 (d, J=8.3 Hz, 1H), 7.26-7.33 (m, 2H). LRMS m/z (APCl⁺) 310.3 (M+1).

Preparation 3.5. {2-(4-Methoxy-benzyl)-5-[3-(4-methoxy-benzylamino)-cyclopentyl]-2H-pyrazol-3-yl}-(3-trifluoromethyl-phenyl)-amine

To a stirring slurry of 3-[1-(4-Methoxy-benzyl)-5-(3-trifluoromethyl-phenylamino)-1H-pyrazol-3-yl]-cyclopentanone (Preparation 3.4, 3.3 g, 7.6 mmol) in 76 mL of toluene was added, powdered 4 Å MS (16.1 g), followed by 4-methoxybenzyl amine (2.0 g, 15.3 mmol). The resulting mixture was heated to 110° C. overnight. After 12 hr the reaction was cooled to rt and Na(OAc)₃BH (3.2 g, 15.3 mmol) was added and stirred for an additional hr before 4 Å MS were filtered off and the resulting solution was concentrated under reduced pressure. Purification of this material was accomplished by MPLC using the Biotage Flash 75S system eluting with a gradient of 5% through 8% MeOH/CH₂Cl₂ containing 0.1% NH₄OH. The product-containing fraction was collected and concentrated under reduced pressure to give the title compound (3.9 g, 92% yield) as a yellow viscous oil and a 3:1 mixture of diastereomers.

R_{f} 0.28 (8% MeOH/CH₂Cl₂), 3:2 ratio of cis, trans isomers seen in the ¹HNMR obtained in CD₃OD. LRMS m/z (APCl⁺) 551 (M+1).

### Examples 27 and 28

### Isomers of [5-(3-Benzylamino-cyclopentyl)-1H-pyrazol-3-yl]-(3-trifluoromethyl-phenyl)-amine

A racemate comprising cis:trans isomers of [5-(3-Benzylamino-cyclopentyl)-1H-pyrazol-3-yl]-(3-trifluoromethyl-phenyl)-amine was prepared according to the method for Preparation 3.5, using Example 26 instead of Preparation 3.4. Cis:trans isomers (Examples 27 and 28) were isolated from the mixture by MPLC using the Biotage flash 755 system eluting with a gradient of 5% through 8% MeOH/CH₂Cl₂ containing 0.1% NH₄OH.

### Example 27

¹HNMR (400 MHz, CD₃COCD₃), δ 1.65-1.79 (m, 1H), 1.81-2.07 (m, 4H), 2.25 (m, 1H), 2.50 (dddd, J=7.1, 7.1, 7.1, 7.1 Hz, 1H), 3.24 (dddd, J=2.1, 2.1, 2.1, 2.1 Hz, 1H), 3.80 (s, 2H), 5.70 (s, 1H), 7.01 (d, J=4.3 Hz, 1H), 7.22-7.55 (m, 5H), 7.56 (d, J=2.1 Hz, 1H), 7.98 (s, 1H), 8.01 (s, 1H). LRMS m/z (APCl⁺) 401.3 (M+1).

### Example 28

¹HNMR (400 MHz, CD₃COCD₃), δ 1.57-1.84 (m, 2H), 1.85-1.99 (m, 1H), 2.04-2.23 (m, 3H), 3.34-3.42 (m, 2H), 3.80 (s, 2H), 5.70 (s, 1H), 7.00 (d, J=7.9 Hz, 1H), 7.20-7.55 (m, 5H), 7.56 (d, J=2.0 Hz, 1H), 7.98 (s, 1H), 8.00 (s, 1H). LRMS m/z (APCl⁺) 401.3 (M+1).

### Example 29

### {5-[cis-3-(4-Methoxy-benzylamino)-cyclopentyl]-1H-pyrazol-3-yl}-(3-trifluoromethyl-phenyl)-amine

The title compound was prepared according to the method for preparation 3.5, using the title compound of Example 26 instead of the title compound of Preparation 3.4.

¹HNMR (400 MHz, CD₃OD), δ 1.47-1.56 (m, 1H), 1.60-1.69 (m, 1H), 1.90-1.97 (m, 2H), 2.10-2.18 (m, 2H), 3.28-3.31 (m, 2H), 3.67 (s, 2H), 3.75 (s, 3H), 5.72 (s, 1H), 6.86 (d, J=6.6 Hz, 2H), 6.97 (d, J=7.0 Hz, 1H), 7.25 (d, J=8.7 Hz, 2H), 7.31-7.35 (m, 2H), 7.48 (s, 1H). LRMS m/z (APCl⁺) 431.3 (M+1).

Preparation 3.6. N-(4-Methoxy-benzyl)-N-{3-[1-(4-methoxy-benzyl)-5-(3-trifluoromethyl-phenylamino)-1H-pyrazol-3-yl]-cyclopentyl}-acetamide

To a stirring solution of {2-(4-Methoxy-benzyl)-5-[3-(4-methoxy-benzylamino)-cyclopentyl]-2H-pyrazol-3-yl}-(3-trifluoromethyl-phenyl)-amine (Preparation 3.5, 109 mg, 0.198 mmol) in 0.5 mL of pyridine was added acetic anhydride (94 uL, 0.99 mmol). After 30 min the reaction was determined to be complete by TLC analysis. This reaction mixture was concentrated under reduced pressure to give N-(4-Methoxy-benzyl)-N-{3-[1-(4-methoxy-benzyl)-5-(3-trifluoromethyl-phenylamino)-1H-pyrazol-3-yl]-cyclopentyl}-acetamide as a 1:1 ratio of cis, trans isomers determined by ¹HNMR and as a foam. This material was used without further purification.

R_{f} 0.75 (5% MeOH/CH₂Cl₂); LRMS m/z (APCl⁺) 593 (M+1).

Analytical separation of the four isomers of the title compound was accomplished using the following conditions: Column: Chiralcel OD, 5 cm×10 cm. Mobile phase: 95/5 heptane/EtOH containing 0.025% DEA as a modifier. Flow rate: 75 mL/min. The sample was loaded using 1:1 Methylene chloride/mobile phase. The retention times for the four isomers were 30 min, 37 min, 45 min, and 60 min, respectively.

### Example 30

### N-{cis-3-[5-(3-Trifluoromethyl-phenylamino)-2H-pyrazol-3-yl]-cyclopentyl}-acetamide

To N-(4-Methoxy-benzyl)-N-{3-[1-(4-methoxy-benzyl)-5-(3-trifluoromethyl-phenylamino)-1H-pyrazol-3-yl]-cyclopentyl}-acetamide (Preparation 3.6, 0.20 mmol 140 mg) was added 2 mL of TFA and reaction was heated to 76° C. After 72 h the reaction cooled to rt and concentrated under reduced pressure. Purification of this material was accomplished by MPLC using a 10 g ISCO™ column eluting with 5% MeOH/CH₂Cl₂. The product-containing fractions were collected and concentrated to give the title compound (60 mg, 86% yield over two steps).

R_{f} 0.30 (55 MeOH/CH₂Cl₂); ¹HNMR (400 MHz, CD₃COCD₃), δ 1.55-1.59 (m, 1H), 1.66-1.74 (m, 1H), 1.83 (s, 3H), 1.97-2.03 (m, 2H), 2.09-2.20 (m, 2H), 3.32 (dddd, J=8.3, 8.3, 8.3, 8.3 Hz, 1H), 4.33 (dddd, J=12.9, 7.0, 7.0, 7.0 Hz, 1H), 5.71 (s, 1H), 7.01 (d, J=7.9 Hz, 1H), 7.16 (bs, 1H), 7.36 (dd, J=7.9, 7.9 Hz, 1H), 7.55 (d, J=8.3 Hz, 1H), 7.99 (bs, 1H), 8.01 (bs, 1H), LRMS m/z (APCl⁺) 353.2 (M+1).

### Example 31

### Pyridine-2-carboxylic acid {3-[5-(3-trifluoromethyl-phenylamino)-2H-pyrazol-3-yl]-cyclopentyl}-amide

The title compound was prepared according to the method for Example 30, using analogous reactants.

R_{f} 0.33 (5% MeOH/CH₂Cl₂), 1:1 ratio of cis, trans isomers seen in ¹HNMR obtained in CD₃OD. LRMS m/z (APCl⁺) 416 (M+1), mp 180.1° C. (HCl salt)

### Example 32

### Pyridine-2-carboxylic acid {3-[5-(3-trifluoromethyl-phenylamino)-2H-pyrazol-3-yl]-cyclopentyl}-amide

The title compound was prepared according to the method for Example 30, using analogous reactants.

R_{f} 0.28 (5% MeOH/CH₂Cl₂), 1:1 ratio of cis, trans isomers seen in ¹HNMR obtained in CD₃OD. LRMS m/z (APCl⁺) 483 (M+1), mp 162.5° C. (HCl salt)

### Example 33

### Cyclobutanecarboxylic acid {3-[5-(3-trifluoromethyl-phenylamino)-2H-pyrazol-3-yl]-cyclopentyl}-amide

The title compound was prepared according to the method for Example 30, using analogous reactants.

R_{f} 0.25 (5% MeOH/CH₂Cl₂), 1:1 ratio of cis, trans isomers seen in ¹HNMR obtained in CD₃OD. LRMS m/z (APCl+) 393 (M+1), mp 232.4° C. (HCl salt)

### Example 34

### 2,2-Dimethyl-N-{3-[5-(3-trifluoromethyl-phenylamino)-2H-pyrazol-3-yl]-cyclopentyl}-propionamide

The title compound was prepared according to the method for Example 30, using analogous reactants.

R_{f} 0.23 (5% MeOH/CH₂Cl₂), 1:1 ratio of cis, trans isomers seen in ¹HNMR obtained in CD₃OD. LRMS m/z (APCl⁺) 395 (M+1), mp 249.2° C. (HCl salt)

### Example 35

### 4-Fluoro-N-{3-[5-(3-trifluoromethyl-phenylamino)-2H-pyrazol-3-yl]-cyclopentyl}-benzamide

The title compound was prepared according to the method for Example 30, using analogous reactants.

R_{f} 0.30 (5% MeOH/CH₂Cl₂), 1:1 ratio of cis, trans isomers seen in ¹HNMR obtained in CD₃OD. LRMS m/z (APCl⁺) 433 (M+1); mp (dec), no distinct melting point.

### Example 36

### 2,2,2-Trifluoro-N-{3-[5-(3-trifluoromethyl-phenylamino)-2H-pyrazol-3-yl]-cyclopentyl}-acetamide

The title compound was prepared according to the method for Example 30, using analogous reactants.

R_{f} 0.30 (5% MeOH/CH₂Cl₂), 3:2 ratio of cis, trans isomers seen in ¹HNMR obtained in CDCl₃. LRMS m/z (APCl⁺) 407 (M+1).

### Example 37

### Cyclopropanecarboxylic acid {3-[5-(3-trifluoromethyl-phenylamino)-2H-pyrazol-3-yl]-cyclopentyl}-amide

The title compound was prepared according to the method for Example 30, using analogous reactants.

R_{f} 0.28 (8% MeOH/CH₂Cl₂), 3:2 ratio of cis, trans isomers seen in ¹HNMR obtained in CDCl₃. LRMS m/z (APCl⁺) 379 (M+1).

### Example 38

### N-{3-[5-(3-Trifluoromethyl-phenylamino)-2H-pyrazol-3-yl]-cyclopentyl}-propionamide

The title compound was prepared according to the method for Example 30, using analogous reactants.

R_{f} 0.30 (8% MeOH/CH₂Cl₂), 3:2 ratio of cis, trans isomers seen in ¹HNMR obtained in CDCl₃. LRMS m/z (APCl⁺) 367 (M+1).

### Example 39

### Cyclohexanecarboxylic acid {3-[5-(3-trifluoromethyl-phenylamino)-2H-pyrazol-3-yl]-cyclopentyl}-amide

The title compound was prepared according to the method for Example 30, using analogous reactants.

R_{f} 025 (8% MeOH/CH₂Cl₂), 3:2 ratio of cis, trans isomers seen in ¹HNMR obtained in CDCl₃. LRMS m/z (APCl⁺) 421 (M+1).

Preparation 4.1. (1,4-Dioxa-spiro[4.4]non-7-yl)-oxo-acetonitrile

To 9 mL of THF at −78° C. was added nBuLi (3.4 mL, 8.6 mmol, 2.5 M in hexanes). After reaction temperature equilibrated (˜15 min), acetonitrile (449 uL, 359 mg, 8.6 mmol) was added dropwise. Reaction was allowed to stir 1 hr before a solution of 1,4-Dioxa-spiro[4.4]nonane-7-carboxylic acid methyl ester (723 mg, 4.3 mmol) was added down the side of the flask. After 1 hr the reaction was warmed to −45° C. (acetonitrile/CO₂) and allowed to stir for 2 hr. Reaction was quenched cold by the dropwise addition of 2 N HCl (˜4.3 mL), pH=7 and then diluted with Et₂O. The layers were separated and the organic layer was dried over MgSO₄, filtered and concentrated under reduced pressure to yield the title compound as a brown oil that was used without further purification.

R_{f} 0.19 (50% EtOAc/hexanes); LRMS m/z (APCl⁺) 196 (M+1).

Preparation 4.2. 5-(1,4-Dioxa-spiro[4.4]non-7-yl)-2-(4-methoxy-benzyl)-2 2H-pyrazol-3-ylamine

To crude (1,4-Dioxa-spiro[4.4]non-7-yl)-oxo-acetonitrile (Preparation 4.1) in EtOH (6.8 mL) was added 4-methoxy-benzyl-hydrazine (0.60 g, 4.0 mmol), and then the reaction was heated to 65° C. After 2 ½ hr the reaction was cooled to rt and concentrated under reduced pressure. Purification of this material was accomplished by MPLC using the Biotage Flash 40L system eluting with a gradient of 50% through 100% EtOAc/hexanes, collecting 18 mm fractions. The product containing-fraction was collected and concentrated under reduced pressure to give the title compound (0.83 g, 75% yield over 2 steps).

R_{f} 0.13 (50% EtOAc/hexanes); ¹HNMR (400 MHz, CDCl₃), δ 2.10-1.72 (m, 5H), 2.23 (dd, J=13.3, 7.9 Hz, 1H), 3.15 (dddd, J=7.9, 7.9, 2.5, 2.5 Hz, 1H), 3.39 (br s, 2H), 3.73 (s, 3H), 3.91-3.84 (m, 4H), 5.03 (s, 2H), 5.34 (s, 1H), 6.80 (d, J=8.7 Hz, 2H), 7.04 (d, J=8.6 Hz, 2H); ¹³C NMR (100 Mz, CDCl₃) δ 31.4, 36.4, 37.2, 43.1, 51.2, 55.5, 64.3, 64.5, 89.0, 114.4, 117.9, 128.3, 129.1, 145.2, 155.0, 159.3; LRMS m/z (APCl⁺) 330 (M+1).

Preparation 4.3. N-[5-(1,4-Dioxa-spiro[4.4]non-7-yl)-2-(4-methoxy-benzyl)-2H-pyrazol-3-yl]-2-naphthalen-1-yl-acetamide

To a stirring solution of 5-(1,4-Dioxa-spiro[4.4]non-7-yl)-2-(4-methoxy-benzyl)-2H-pyrazol-3-ylamine (Preparation 4.2, 0.83 g, 2.5 mmol) in CH₂Cl₂ (5 mL) was added a freshly prepared solution of naphthalen-1-yl-acetyl chloride (1.03 g, 5.0 mmol in CH₂Cl₂) followed by the addition of 1 mL of pyridine. After 2 hr, the reaction was quenched with H₂O, and 2 mL of a solution of NH₄OH (15%) was added. This mixture was diluted with CH₂Cl₂ and the layers were separated. The organic layer was dried over MgSO₄, filtered and concentrated under reduced pressure. Purification of this material was accomplished by MPLC using the Biotage Flash 40L system eluting with a gradient of 25% through 50% acetone/hexanes, collecting 18 mm fractions. The product-containing fraction was collected and concentrated under reduced pressure to give the title compound (1.2 g, 97% yield) as a light yellow solid.

mp 162.8° C.; ¹HNMR (400 MHz, CDCl₃), δ 2.09-173 (m, 5H), 2.21 (dd, J=13.3, 7.9 Hz, 1H), 3.18 (dddd, J=7.5, 5.0 Hz, 1H), 3.68 (s, 3H), 3.89-3.81 (m, 4H), 3.98 (s, 2H), 4.60 (s, 2H), 6.22 (s, 1H), 6.36 (d, J=8.7 Hz, 2H), 6.51 (d, J=8.7 Hz, 2H), 7.03 (br s, 1H), 7.23 (d, J=6.7 Hz, 1H), 7.39 (dd, J=7.1, 7.1 Hz, 1H), 7.53-7.45 (m, 2H), 7.88-7.83 (m, 2H), 7.89 (d, J=1.7 Hz, 1H); ¹³C NMR (100 Mz, CDCl₃) δ 31.2, 36.4, 37.2, 42.0, 42.9, 51.9, 55.4, 64.3, 64.5, 97.2, 114.2, 117.8, 123.7, 125.9, 126.7, 127.4, 127.7, 127.9, 128.7, 129.1, 129.32, 130.2, 132.1, 134.2, 135.3, 154.9, 159.2, 168.6; LRMS m/z (APCl⁺) 498 (M+1).

Preparation 4.4. N-[2-(4-Methoxy-benzyl)-5-(3-oxo-cyclopentyl)-2H-pyrazol-3-yl]-2-naphthalen-1-yl-acetamide

The title compound was synthesized according to the method for Preparation 3.4, using the title compound of Preparation 4.3 instead of the title compound of Preparation 3.3 as a reactant.

¹HNMR (400 MHz, CDCl₃), δ 1.99-2.18 (m, 1H), 2.21-2.28 (m, 1H), 2.35-2.45 (m, 3H), 2.53-2.62 (m, 1H), 3.43 (dddd, J=9.5, 6.2, 7.9, 9.5 Hz, 1H), 3.75 (s, 3H), 4.12 (s, 2H), 4.62 (s, 2H), 6.30 (s, 1H), 6.33 (d, J=9.5 Hz, 1H), 6.55 (d, J=8.7 Hz, 1H), 7.31-7.33 (m, 1H), 7.45-7.48 (m, 1H), 7.56-7.60 (m, 2H), 7.89-7.97 (m, 3H). LRMS m/z (APCl⁺) 454 (M+1).

Preparation 4.5. N-{2-(4-Methoxy-benzyl)-5-[3-(4-methoxy-benzylamino)-cyclopentyl]-2H-pyrazol-3-yl}-2-naphthalen-1-yl-acetamide

The title compound was prepared according to the method for Preparation 3.5, using the title compound of Preparation 4.4 as a reactant instead of the title compound of Preparation 3.4.

R_{f} 0.25 (8% MeOH/CH₂Cl₂) (minor), R_{f} 0.20 (8% MeOH/CH₂Cl₂) (major), 3:2 ratio of cis, trans isomers seen in ¹HNMR obtained in CDCl₃. LRMS m/z (APCl⁺) 575 (M+1).

Preparation 4.6. N-[5-{3-[Acetyl-(4-methoxy-benzyl)-amino]-cyclopentyl}-2-(4-methoxy-benzyl)-2H-pyrazol-3-yl]-2-naphthalen-1-yl-acetamide

The title compound was synthesized according to the method for Preparation 3.6, using the title compound of Preparation 4.5 instead of the title compound of Preparation 3.5 as a reactant.

R_{f} 0.30 (80% EtOAc/Toluene), R_{f} 0.25 (80% EtOAc/Toluene), 1:1 ratio of cis, trans isomers seen in ¹HNMR obtained in CDCl₃.

Enantiomeric separation of the isomer with the R_{f} 0.25 was accomplished using the following conditions: Column: Chiralcel OD, 5 cm×50 cm. Mobile phase: 60/40 Heptane/EtOH containing 0.025% DEA as a modifier. Flow rate: 50 mL/min. The sample was loaded using methanol. The retention times for the two enantiomers were 35 min and 45 min. LRMS m/z (APCl⁺) 617 (M+1).

Preparation 5.1. Cyclobutanecarboxylic acid (4-methoxy-benzyl)-{3-[5-(3-trifluoromethyl-phenylamino)-2H-pyrazol-3-yl]-cyclopentyl}-amide

The title compound was prepared according to the method for Preparation 4.6, using analogous reactants.

R_{f} 0.45 (5% MeOH/CH₂Cl₂), 1:1 ratio of cis, trans isomers seen in ¹HNMR obtained in CDCl₃. LRMS m/z (APCl⁺) 633 (M+1).

### Example 40

### N-[5-(3-Acetylamino-cyclopentyl)-2H-pyrazol-3-yl]-2-naphthalen-1-yl-acetamide

The title compound was synthesized by the method for Example 30, using Preparation 4.6 as a reactant instead of Preparation 3.6.

R_{f} 0.30 (8% MeOH/CH₂Cl₂). 1:1 ratio of cis, trans isomers was seen in the ¹HNMR obtained in CD₃OD. LRMS m/z (APCl⁺) 377 (M+1).

### Example 41

### Cyclopropanecarboxylic acid {3-[5-(2-naphthalen-1-yl-acetylamino)-1H-pyrazol-3-yl]-cyclopentyl)-amide

The title compound was synthesized by the method for Example 30, using analogous reactants.

R_{f} 0.30 (5% MeOH/CH₂Cl₂). 1:1 ratio of cis, trans isomers was seen in the ¹HNMR obtained in CDCl₃. LRMS m/z (APCl⁺) 403 (M+1).

### Example 42

### 2-Naphthalen-1-yl-N-5-[3-(2,2,2-trifluoro-acetylamino)-cyclopentyl]-2H-pyrazol-3-yl}-acetamide

The title compound was synthesized by the method for Example 30, using analogous reactants.

R_{f} 0.30 (5% MeOH/CH₂Cl₂). 1:1 ratio of cis, trans isomers was seen in the ¹HNMR obtained in CD₃OD. LRMS m/z (APCl⁺) 431 (M+1).

### Example 43

### N-{3-[5-(2-Naphthalen-1-yl-acetylamino)-1H-pyrazol-3-yl]-cyclopentyl}-benzamide

The title compound was synthesized by the method for Example 30, using analogous reactants.

R_{f} 0.30 (5% MeOH/CH₂Cl₂). 1:1 ratio of cis, trans isomers was seen in the ¹HNMR obtained in CDCl₃. LRMS m/z (APCl⁺) 439 (M+1).

The following title compounds of Examples 44-57 were synthesized by preparing an intermediate analogous to the title compound of Preparation 4.6, using the steps described in “Preparation 4.2” through “Preparation 4.5”. In each case, a reactant analogous to naphthalen-1-yl acetal chloride used in synthesis of the title compound of Preparation 4.3 was used. This intermediate was than treated according to the method described for synthesis of the title compound of Example 30:

### Example 44

### 3-Methoxy-N-{cis-3-[5-(2-naphthalen-1-yl-acetylamino)-2H-pyrazol-3-yl]-cyclobutyl}-benzamide

R_{f} 0.33 (10% MeOH/CH₂Cl₂); mp 124.8° C. (mono HCl salt); ¹HNMR (400 MHz, CD₃OD), δ 2.20 (dd, J=10.2 Hz, 2H), 2.72-2.66 (m, 2H), 3.14 (apt quint, J=8.8 Hz, 1H), 3.75 (s, 3H), 4.12 (s, 2H), 4.40 (apt quint, 8.4 Hz, 1H), 6.31 (s, 1H), 7.03-7.00 (m, 1H), 7.28 (dd, J=7.8 Hz, 1H), 7.46-7.35 (m, 6H), 7.74 (d, J=7.9 Hz, 1H), 7.81 (d, J=7.0 Hz, 1H), 8.01 (d, J=7.4 Hz, 1H); ¹³C NMR (100 Mz, CD₃OD) δ 170.6, 168.1, 160.0, 149.0, 146.0, 135.5, 134.2, 132.4, 131.3, 129.4, 128.6, 128.1, 127.9, 126.2, 125.7, 125.4, 123.7, 119.3, 117.3, 112.5, 93.8, 54.7, 41.5, 40.2, 36.9, 24.8; LRMS m/z (APCl⁺) 455.1 (M+1).

### Example 45

### N-{cis-3-[5-(2-Naphthalen-1-yl-acetylamino)-2H-pyrazol-3-yl]-cyclobutyl}-3-trifluoromethyl-benzamide

R_{f} 0.56 (10% MeOH/CH₂Cl₂); mp 142.8° C. (mono HCl salt); ¹HNMR (400 MHz, CD₃OD), δ 2.22 (dd, J=9.5, 9.5 Hz, 2H), 2.73 (dd, J=7.4, 7.4 Hz, 2H), 3.18 (apt quint, J=7.9 Hz, 1H), 4.14 (s, 2H), 4.46 (apt quint, J=7.5 Hz, 1H), 6.40 (s, 1H), 7.48-4.37 (m, 4H), 7.83 (d, J=4.9 Hz, 1H), 8.03 (s, 1H), 8.05 (s, 1H), 8.13 (s, 1H); ¹³C NMR (100 Mz, CD₃OD) δ 170.5, 166.4, 148.7, 146.5, 135.2, 134.2, 132.5, 131.5, 130.9, 130.5, 129.3, 128.5, 128.0, 127.8, 126.2, 125.6, 125.4, 124.1, 124.0, 123.7, 94.0, 41.6, 40.3, 36.9, 24.9; LRMS m/z (APCl⁺) 493.0 (M+1).

### Example 46

### N-{cis-3-[5-(2-Naphthalen-1-yl-acetylamino)-2H-pyrazol-3-yl]-cyclobutyl}-isobutyramide

R_{f} 0.43 (10% MeOH/CH₂Cl₂ with 0.1% NH₄OH aq); mp 130.6° C. (mono HCl salt);

¹HNMR (400 MHz, d6 DMSO), δ 0.93 (s, 3H), 0.95 (s, 3H), 1.92 (dd, J=9.9, 9.9 Hz, 2H), 2.24 (apt quint, J=6.8 Hz, 1H), 2.54,-2.48 (m, 2H), 3.01 (apt quint, J=8.3 Hz, 1H), 4.08 (s, 2H), 4.15-4.08 (m, 1H), 6.32 (s, 1H), 7.54-7.43 (m, 4H), 7.81 (d, J=7.4 Hz, 1H), 7.94-7.89 (m, 2H), 8.13 (d, J=7.9 Hz, 1H); ); ¹³C NMR (100 Mz, d6 DMSO) δ 175.7, 168.7, 147.7, 134.0, 132.6, 129.0, 128.5, 127.8, 126.7, 126.3, 126.2, 125.0, 94.0, 39.6, 38.2, 34.6, 24.5, 20.2; LRMS m/z (APCl⁺) 391 (M+1).

### Example 47

### 2-Phenyl-cyclopropanecarboxylic acid {cis-3-[5-(2-naphthalen-1-yl-acetylamino)-2H-pyrazol-3-yl]-cyclobutyl}-amide

R_{f} 0.56 (10% MeOH/CH₂Cl₂ with 0.1% NH₄OH aq); mp 139.8° C. (mono HCl salt); ¹HNMR (400 MHz, d6 DMSO), δ 1.17-1.14 (m, 1H), 1.32-1.28 (m, 1H), 1.75-1.70 (m, 1H), 1.97-1.89 (dd, J=10.4, 10.4, 2H), 2.20-2.16 (m, 1H), 2.55-2.49 (m, 2H), 3.02 (apt, quint, J=8.2 Hz, 1H), 4.07 (s, 2H), 4.17 (m, 1H), 6.30 (s, 1H), 7.23-7.07 (m, 5H), 7.54-7.42 (m, 4H), 7.80 (d, J=7.9, 1H), 7.90 (d, J=7.9 Hz, 1H), 8.12 (d, J=7.8 Hz, 1H), 8.37 (d, J=7.8 Hz, 1H); ¹³C NMR (100 MHz, d6 DMSO) δ 172.9, 170.8, 152.5, 142.8, 140.8, 134.2, 132.4, 130.3, 129.0, 128.6, 128.3, 126.3, 126.1, 125.9, 125.8, 125.4, 123.6, 92.9, 41.1, 36.5, 25.5, 24.8, 24.2, 15.1; LRMS m/z (APCl⁺) 465.0 (M+1).

### Example 48

### N-[5-(cis-3-Acetylamino-cyclobutyl)-1H-pyrazol-3-yl]-2-naphthalen-1-yl-acetamide

R_{f} 0.50 (10% MeOH/CH₂Cl₂). ¹HNMR (400 MHz, CD₃OD), δ 1.86 (s, 3H), 1.97-2.05 (m, 2H), 2.59-2.66 (m, 2H), 3.07 (apt quint, J=8.0 Hz, 1H), 4.13 (s, 2H), 4.20 (apt quint, J=7.8 Hz, 1H), 6.25 (s, 1H), 7.37-7.48 (m, 4H), 7.77 (d, J=7.9 Hz, 1H), 7.81-7.85 (m, 1H), 8.01-8.03 (m, 1H). ¹³C-NMR: 21.37, 24.64, 37.09, 40.20, 40.78, 93.59, 93.65, 123.71, 125.41, 125.70, 126.22, 127.88, 128.04, 128.58, 131.36, 132.45, 134.18, 146.36, 148.68, 170.53, 171.31.). MS LRMS m/z (APCl⁺) 363 (M+1), mp 209.4° C. (HCl salt).

### Example 49

### N-{cis-3-[5-(2-Naphthalen-1-yl-acetylamino)-2H-pyrazol-3-yl]-cyclobutyl}-benzamide

R_{f} 0.50 (10% MeOH/CH₂Cl₂). ¹HNMR (400 MHz, CD₃OD), δ 2.20-2.30 (m, 2H), 2.70-2.80 (m, 2H), 3.20 (apt quint, J=8.0 Hz, 1H), 4.18 (s, 2H), 4.48 (apt quint, J=7.8 Hz, 1H), 6.24 (bs, 1H), 7.38-7.50 (m, 7H), 7.75-7.88 (m, 4H), 8.00-8.05 (m, 1H). ¹³C-NMR: 24.68, 36.92, 40.12, 41.47, 93.54, 123.68, 125.39, 125.72, 126.25, 127.20, 127.96, 128.12, 128.35, 128.59, 131.17, 131.56, 132.44, 134.20, 145.86, 149.19, 168.39, 170.60. MS LRMS m/z (APCl⁺) 425 (M+1), mp 194.0° C.(HCl salt).

### Example 50

### 2-Cyclopropyl-N-{cis-3-[5-(2-naphthalen-1-yl-acetylamino)-2H-pyrazol-3-yl]-cyclobutyl}-acetamide

R_{f} 0.60 (10% MeOH/CH₂Cl₂). ¹HNMR (400 MHz, CD₃OD), δ 0.93 (t, J=7.5 Hz, 2H), 1.34 (d, J=6.2 Hz, 1H), 1.65 (d, J=5.0 Hz, 1H), 1.70-1.78 (m, 2H), 2.01-2.15 (m, 2H), 2.55-2.58 (m, 1H), 2.62-2.68 (m, 2H), 3.20 (apt quint, J=7.8 Hz, 1H), 4.20 (s, 2H), 5.40 (apt quint, J=6.2 Hz, 1H), 6.22 (bs, 1H), 7.40-7.56 (m, 4H), 7.78-7.88 (m, 2H), 8.02-8.08 (m, 1H), ¹³C-NMR: 8.27, 24.52, 26.64, 36.63, 36,85, 36.96, 39.47, 40.12, 40.77, 77.66, 93.27, 112.50, 123.67, 125.38, 125.73, 126.24, 127.95, 128.14, 128.59, 132.45, 134.21, 146.71, 169.62, 170.63. MS LRMS m/z (APCl⁺) 403 (M+1), mp 93.2° C. (HCl salt).

### Example 51

### 6-Chloro-pyridine-2-carboxylic acid {cis-3-[5-(2-naphthalen-1-yl-acetylamino)-2H-pyrazol-3-yl]-cyclobutyl}-amide

R_{f} 0.50 (10% MeOH/CH₂Cl₂). ¹HNMR (400MHz, CD₃OD), δ 2.25-2.42 (m, 2H), 2.76-2.82 (m, 2H), 3.30 (apt quint, J=7.9 Hz, 1H), 4.22 (s, 2H), 4.54 (apt quint, J=7.5 Hz, 1H), 6.35 (s, 1H), 7.42-7.64 (m, 5H), 7.81-7.89 (m, 2H), 7.93-8.11 (m, 3H). ¹³C-NMR: 24.58, 36.65, 40.17, 41.06, 77.50, 93.77, 121.03, 122.58, 123.67, 123.82, 124.50, 125.40, 125.72, 126.24, 127.37, 128.01, 128.11, 128.59, 134.20, 140.63, 140.70, 144.41, 150.36, 170.70, 182.15. MS LRMS m/z (APCl⁺) 460 (M+1), mp (dec) (HCl salt).

### Example 52

### Quinoline-2-carboxylic acid {cis-3-[5-(2-naphthalen-1-yl-acetylamino)-2H-pyrazol-3-yl]-cyclobutyl}-amide

R_{f} 0.30 (5% MeOH/CH₂Cl₂). ¹HNMR (400 MHz, CD₃OD), δ 2.25-2.38 (m, 2H), 2.76-2.78 (m, 2H), 3.25 (apt quint, J=8.3 Hz, 1H), 4.16 (s, 2H), 4.56 (apt quint, 8.7 Hz, 1H), 6.36 (bs, 1H), 7.38-7.49 (m, 4H), 7.60-7.64 (m, 1H), 7.71-7.99 (m, 4H), 8.02-8.13 (m, 3H), 8.36 (d, J=8.7 Hz, 1H). ¹³C-NMR: 24.80, 37.08, 40.22, 41.08, 53.50, 94.03, 118.41, 123.70, 124.50, 125.39, 125.70, 126.22, 127.25, 127.82, 128.15, 128.57, 129.42, 129.54, 130.34, 132.43, 134.18, 137.70, 137.80, 146.02, 146.73, 148.93, 149.60, 164.89, 170.57. MS LRMS m/z (APCl⁺) 476 (M+1), mp 200.3° C. (HCl salt).

### Example 53

### Pyrazine-2-carboxylic acid {cis-3-[5-(2-naphthalen-1-yl-acetylamino)-2H-pyrazol-3-yl]-cyclobutyl}-amide

R_{f} 0.30 (5% MeOH/CH₂Cl₂). ¹HNMR (400 MHz, CD₃OD), δ 2.25-2.38 (m, 2H), 2.75-2.84 (m, 2H), 3.26 (apt quint, J=8.7 Hz, 1H), 4.21 (s, 2H), 4.55 (apt quint, J=8.7 Hz, 1H), 6.28 (s, 1H), 7.42-7.54 (m, 4H), 7.81-7.89 (m, 2H), 8.03-8.07 (m, 1H), 8.66 (d, J=2.5 Hz, 1H), 8.75 (d, J=2.5 Hz, 1H), 9.20 (s, 1H). MS LRMS m/z (APCl⁺) 427 (M+1).

### Example 54

### 4-Methoxy-N-{cis-3-[5-(2-naphthalen-1-yl-acetylamino)-2H-pyrazol-3-yl]-cyclobutyl}-benzamide

R_{f} 0.35 (5% MeOH/CH₂Cl₂). ¹HNMR (400 MHz, CD₃OD), δ 2.17-2.25 (m, 2H), 2.68-2.75 (m, 2H), 3.18 (apt quint, J=8.3 Hz, 1H), 3.79 (s, 3H), 4.15 (s, 2H), 4.44 (apt quint, J=7.8 Hz, 1H), 6.33 (s, 1H), 6.91 (d, J=5.0 Hz, 2H), 7.37-7.49 (m, 4H), 7.77 (d, J=5.0 Hz, 2H), 7.82-7.84 (m, 1H), 7.96-8.04 (m, 2H). ¹³C-NMR: 24.80, 37.02, 40.22, 41.46, 54.71, 54.76, 93.90, 113.50, 123.72, 125.41, 125.70, 126.22, 127.24, 127.88, 128.08, 128.58, 129.09, 131.36, 132.44, 134.17, 142.17, 146.03, 146.21, 149.14, 162.76, 167.93, 170.56. MS LRMS m/z (APCl⁺) 455 (M+1), mp 175.6° C. (HCl salt).

### Example 55

### N-{cis-3-[5-(2-Naphthalen-1-yl-acetylamino)-2H-pyrazol-3-yl]-cyclobutyl}-3-nitro-benzamide

R_{f} 0.35 (5% MeOH/CH₂Cl₂). ¹HNMR (400 MHz, CD₃OD), δ 2.26-2.34 (m, 2H), 2.74-2.79 (m, 2H), 3.25 (apt quint, J=8.7 Hz, 1H), 4.19 (bs, 2H), 4.50 (apt quint, J=8.3 Hz, 1H), 6.31 (bs, 1H), 7.37-7.49 (m, 4H), 7.63-7.80 (m, 3H), 8.19-8.33 (m, 2H), 8.58-8.65 (m, 2H), ¹³C-NMR: 24.82, 36.80, 40.10, 41.73, 93.87, 115.43, 118.34, 122.22, 122.28, 123.81, 125.87, 126.27, 127.38, 127.87, 128.56, 129.81, 131.39, 132.40, 133.22, 134.06, 135.89, 141.96, 142.04, 146.50, 161.89, 165.60. MS LRMS m/z (APCl⁺) 470 (M+1), mp 123.5° C. (HCl salt).

### Example 56

### 3,5-Dimethoxy-N-{cis-3-[5-(2-naphthalen-1-yl-acetylamino)-2H-pyrazol-3-yl]-cyclobutyl}-benzamide

R_{f} 0.50 (10% MeOH/CH₂Cl₂). ¹HNMR (400 MHz, CD₃OD), δ 2.23-2.30 (, 2H), 2.73-2.79 (m, 2H), 3.23 (apt quint, J=8.3 Hz, 1H), 3.78 (s, 6H), 4.19 (bs, 2H), 4.47 (apt quint, J=8.3 Hz, 1H), 6.30 (bs, 1H), 6.62 (s, 1H), 6.97 (s, 2H), 7.41-7.52 (m, 4H), 7.85 (m, 2H), 8.03-8.08 (m, 1H). MS LRMS m/z (APCl⁺) 485 (M+1)

### Example 57

### 4-Dimethylamino-N-{cis-3-[5-(2-naphthalen-1-yl-acetylamino)-2H-pyrazol-3-yl]-cyclobutyl}-benzamide

R_{f} 0.45 (10% MeOH/CH₂Cl₂). ¹HNMR (400 MHz, CD₃OD), δ 2.18-2.28 (m, 2H), 2.70-2.80 (m, 2H), 2.99 (s, 6H), 3.20 (apt quint, J=8.2 Hz, 1H), 4.18 (bs, 2H), 4.46 (apt quint, J=8.3 Hz, 1H), 6.31 (bs, 1H), 6.69 (d, J=8.7 Hz, 2H), 7.42-7.53 (m, 4H), 7.71 (d, J=9.1 Hz, 2H), 7.80-7.88 (m, 2H), 8.06 (d, J=8.3 Hz, 1H). MS LRMS m/z (APCl⁺) 485 (M+1).

### Example 58

### N-[5-((1S)-Hydroxy-ethyl)-2H-pyrazol-3-yl]-2-naphthalen-1-yl-acetamide

Synthesis of the title compound was accomplished by the method for the title compound of Preparation 4.3, using a reactant analogous to naphthalen-1-yl-acetyl chloride. This was followed by deprotection. The resulting intermediate was treated according to the method for Example 30.

R_{f} 0.50 (10% MeOH/CH₂Cl₂). ¹HNMR (400 MHz, CDCl₃), δ 1.64 (d, J=6.6 Hz, 3H), 4.12 (s, 2H), 5.97 (dd, J=6.6, 6.6 Hz, 1H), 6.47 (s, 1H), 7.34-7.40 (m, 2H), 7.42-7.50 (m, 2H), 7.74-7.87 (m, 3H). LRMS m/z (APCl⁺) 296 (M+1); mp 101.3° C. (HCl salt).

### Example 59

### N-[5-(2-Hydroxy-(1S)-methyl-ethyl)-2H-pyrazol-3-yl]-2-naphthalen-1-yl-acetamide

Synthesis of the title compound was accomplished by the method for Preparation 4.3, using a reactant analogous to naphthalen-1-yl-acetyl chloride. This was followed by deprotection. The resulting intermediate was treated according to the method for Example 30.

¹HNMR (400 MHz, CD₃OD), δ 1.25 (d, J=7.1 Hz, 3H), 2.92-3.01 (m, 1H), 3.58-3.62 (m, 2H), 4.20 (bs, 2H), 6.22 (bs, 1H), 7.40-7.58 (m, 4H), 7.80 (d, J=7.0 Hz, 1H), 7.88 (d, J=7.1 Hz, 1H), 8.06 (d, J=7.0 Hz, 1H). LRMS m/z (APCl⁺) 310 (M+1), mp 117.6° C. (HCl salt).

Preparation 6.1. N-[5-[1-(Benzothiazol-2-yloxy)-ethyl]-2-(4-methoxy-benzyl)-2H-pyrazol-3-yl]-2-naphthalen-1-yl-acetamide

To a stirring solution of N-[5-(1-Hydroxy-ethyl)-2-(4-methoxy-benzyl)-2H-pyrazol-3-yl]-2-naphthalen-1-yl-acetamide (300 mg, 0.72 mmol) in 7.2 mL of THF was added 2-chlorobenzthiozole (104 uL, 0.79 mmol, 135 mg) followed by a solution of KOBu¹ (1.4 mL, 1.4 mmol, 1.0 M in THF) dropwise. After 2 hrs the reaction was quenched with NH₄Cl and then diluted with EtOAc. The layers were separated and the organic layer was dried of MgSO₄, filtered and concentrated under reduced pressure. Purification of this material was accomplished by MPLC using a 10 g ISCO cartridge on a Biotage system eluting with 30% EtOAc/hexanes, collecting 8 mm fractions. The product-containing fraction was collected and concentrated under reduced pressure to give the title compound (100 mg, 25% yield) as a yellow viscous oil.

R_{f} 0.50 (50% EtOAc/Hexanes). ¹HNMR (400 MHz, CDCl₃), δ 1.82 (d, J=7.5 Hz, 3H), 3.74 (s, 3H), 4.06 (d, J=4.6 Hz, 2H), 4.65 (dd, J=15.8, 15.8 Hz, 2H), 6.02 (dd, J=7.1, 7.1 Hz 1H), 6.27 (s, 1H), 6.29 (d, J=8.7 Hz, 2H), 6.51 (d, J=8.7 Hz, 2H), 6.99-7.06 (m, 3H), 7.27-7.29 (m, 1H), 7.30-7.38 (m, 1H), 7.40-7.43 (m, 1H), 7.50-7.60 (m, 2H), 7.87-7.95 (m, 3H). LRMS m/z (APCl⁺) 549 (M+1).

### Example 60

### N-{5-[(1S)-(Benzothiazol-2-yloxy)-ethyl]-1H-pyrazol-3-yl}-2-naphthalen-1-yl-acetamide

The title compound was synthesized using Preparation 6.1 according to the method for Example 15.

R_{f} 0.41 (5% MeOH/CH₂Cl₂). ¹HNMR (400 MHz, CDCl₃), δ 1.81 (d, J=7.1 Hz, 3H), 4.17 (s, 2H), 5.84 (dd, 6.7, 6.7 Hz, 1H), 6.68 (s, 1H), 6.85 (d,J=7.9 Hz, 1H), 7.10-7.20 (m, 2H), 7.32-7.57 (m, 5H), 7.79-7.86 (m, 2H), 7.97-7.99 (m, 1H). LRMS m/z (APCl⁺) 429 (M+1).

### Example 61

### N-[5-(Benzothiazol-2-yloxymethyl)-1H-pyrazol-3-yl]-2-naphthalen-1-yl-acetamide

The title compound was synthesized according to the method for Example 60, using analogous reactants.

R_{f} 0.50 (5% MeOH/CH₂Cl₂). ¹HNMR (400 MHz, CD₃OD), δ 4.20 (s, 2H), 5.38 (s, 2H), 6.46 (s, 1H), 7.40-7.58 (m, 5H), 7.80-7.90 (m, 4H), 8.06-8.08 (m, 2H). LRMS m/z (APCl⁻) 412 (M−1).

### Example 62

### N-{5-[(1R)-(Benzothiazol-2-yloxy)-ethyl]-1H-pyrazol-3-yl}-2-naphthalen-1-yl-acetamide

The title compound was synthesized according to the method for Example 60, using analogous reactants.

R_{f} 0.41 (5% MeOH/CH₂Cl₂). ¹HNMR (400 MHz, CDCl₃), δ 1.85 (d, J=7.1 Hz, 3H), 4.20 (s, 2H), 5.88 (dd, J=7.1, 7.1 Hz, 1H), 6.68 (s, 1H), 6.91 (d, J=7.9 Hz, 1H), 7.15-7.26 (m, 2H), 7.38-7.60 (m, 5H), 7.78-7.95 (m, 3H). LRMS m/z (APCl⁺) 429 (M+1).

### Example 63

### N-{5-[cis-3-(Benzooxazol-2-yloxy)-cyclobutyl]-1H-pyrazol-3-yl}-2-naphthalen-1-yl-acetamide

The title compound was synthesized according to the method for Example 60, using analogous reactants.

R_{f} 0.24 (10% MeOH/CH₂Cl₂); mp 142.0° C. (mono HCl salt); ¹HNMR (400 MHz, CDCl₃) δ 2.43-2.17 (m, 2H), 3.01-2.94 (m, 2H), 3.18 (apt quint, J=8.3 Hz, 1H), 4.12 (s, 2H), 5.21 (apt quint, J=7.3 Hz, 1H), 6.59 (s, 1H), 7.54-7.31 (m, 8H), 7.72 (dd, J=7.9 Hz, 1H), 7.77 (dd, 7.5 Hz, 1H), 7.94 (d, J=8.3 Hz, 1H); ¹³C NMR (100 MHz, CDCl₃) δ 170.3, 162.0, 151.5, 148.4, 143.2, 140.5, 134.0, 132.2, 129.7, 129.0, 128.8, 126.9, 125.8, 125.7, 124.7, 123.8, 123.5, 123.4, 118.1, 110.1, 94.9, 71.3, 41.5, 36.7, 22.8(m, 3H). LRMS m/z (APCl⁺) 439.1 (M+1).

Preparation 7.1. N-[5-(cis-3-hydroxy-3-phenyl-cyclobutyl)-2-(4-methoxy-benzyl)-2H-pyrazol-3-yl]-2-naphthalen-1-yl-acetamide

Preparation of the starting ketone N-[2-(4-methoxy-benzyl)-5-(3-oxo-cyclobutyl)-2H-pyrazol-3-yl]-2-naphthalen-1-yl-acetamide was accomplished using a method analogous to that described for Preparation 4.4 (including synthesis of Preparations 4.1-4.3).

A solution of the ketone (50 mg, 0.11 mmol) in tetrahydrofuran (5 mL) cooled to -30 ° C. was treated dropwise with phenyl magnesium bromide (500 uL, 0.5 mmol, 1M solution in THF). Upon completion of addition, the reaction mixture was stirred for 40 min at −30° C., then saturated aqueous ammonium chloride solution was added and the mixture was warmed to room temperature. THF was removed in vacuo, and the residue was diluted with methylene chloride which was washed with water and brine. The diluted residue was then dried and filtered. The crude material was purified by silica gel chromatography (50:1 chloroform-methanol) to afford 54 mg (80% yield) of the title comopund.

1H NMR (400 MHz, CDCl3) δ 7.9 (m, 3H), 7.55 (m, 4H), 7.46 (m, 1H), 7.35 (m, 3H), 7.21 (m, 1H), 6.90 (m, 1H), 6.80 (dd, J=0.8, 8.7 Hz, 1H), 6.54 (d, J=8.7 Hz, 2H), 6.37 (d, J=8.7 Hz, 2H), 6.34 (s, 1H), 4.65 (s, 2H), 4.12 (s, 2H), 3.74 (s, 3H), 3.20 (m, 1H), 3.01 (m, 2H), 2.55 (m, 2H); MS (AP/Cl): 518.2 (M+H)+.

Preparation 7.2 N-[2-(4-methoxy-benzyl)-5-(cis-3-phenyl-cyclobutyl)-2H-pyrazol-3-yl]-2-naphthalen-1-yl-acetamide

A solution of N-[5-(3-hydroxy-3-phenyl-cyclobutyl)-2-(4-methoxy-benzyl)-2H-pyrazol-3-yl]-2-naphthalen-1-yl-acetamide (Preparation 7.1, 54 mg, 0.10 mmol) in 1:1 methylene chloride-trifluoroacetic acid (4 mL) was treated with triethylsilane (1.2 mL) at 23° C. After stirring for 16 h, the solvent was removed in vacuo and the residue was purified by silica gel chromatography (100:1 chloroform-methanol) to afford 39 mg (78% yield) of the title compound as a 10:1 mixture of cis—trans isomers.

1H NMR (400 MHz, CDCl₃): δ 7.90 (m, 3H), 7.56 (m, 2H), 7.45 (dd, J=7.1, 8.3 Hz, 1H), 7.30 (m, 3H), 7.25 (m, 1H), 7.18 (m, 1H), 6.78 (s, 1H), 6.55 (d, J=8.7 Hz, 2H), 6.36 (m, 3H), 4.63 (s, 2H), 4.10 (s, 2H), 3.74 (s, 3H), 3.44 (m, 2H), 2.73 (m, 2H), 2.30 (m, 2H); MS (AP/Cl): 502.2 (M+H)+; minor isomer, characteristic 1H NMR signals: δ 4.66 (s), 4.12 (s), 2.60 (m).

### Example 64

### 2-naphthalen-1-yl-N-[5-(cis-3-phenyl-cyclobutyl)-1H-pyrazol-3-yl]-acetamide

A solution of N-[2-(4-methoxy-benzyl)-5-(3-phenyl-cyclobutyl)-2H-pyrazol-3-yl]-2-naphthalen-1-yl-acetamide (Preparation 7.2, 38 mg, 0.076 mmol) in trifluoroacetic acid (5 mL) at room temperature was treated with anisole (165 uL, 1.5 mmol). The solution was heated at 70° C. for 5 h. The solvent was removed in vacuo and the residue was purified by silica gel chromatography (40:1 chloroform-methanol) to afford 27 mg (89% yield) of the title compound as a 94:6 mixture of cis—trans isomers. The product was dissolved in ethyl acetate and treated with hydrogen chloride in diethyl ether to afford the HCl salt.

1H NMR (400 MHz, CD3OD): δ 8.04 (d, J=8.3 Hz, 1H), 7.88 (d, J=7.5 Hz, 1H), 7.82 (d, J=7.9 Hz, 1H), 7.50 (m, 4H), 7.28 (m, 4H), 7.17 (m, 1H), 6.26 (s, 1H), 4.27 (s, 2H), 3.59 (m, 2H), 2.82 (m, 2H), 2.30 (m, 2H); MS (AP/Cl): 382.3 (M+H)+.

The title compounds of the following Examples 65-71 were synthesized as in Example 64, including synthesis of Preparations 7.1 and 7.2, using an analogous starting ketone:

### Example 65

### N-{5-[cis-3-(2-Methoxy-phenyl)-cyclobutyl]-2H-pyrazol-3-yl}-2-quinolin-6-yl-acetamide

1H NMR (400 MHz, CDCl₃): δ 9.23 (s, 1H), 8.80 (dd, J=1.7, 4.1 Hz, 1H), 7.93 (m, 2H), 7.54 (d, J=1.7 Hz, 1H), 7.47 (dd, J=2.1, 8.7 Hz, 1H), 7.27 (q, J=4.1 Hz, 1H), 7.15 (m, 1H), 7.08 (d, J=7.5 Hz, 1H), 6.87 (td, J=0.8, 7.5 Hz, 1H), 6.78 (d, J=7.9 Hz, 1H), 6.57 (s, 1H), 3.76 (s, 3H), 3.73 (s, 2H), 3.61 (m, 1H), 3.38 (m, 1H), 2.68 (m, 2H), 2.22 (m, 2H); MS (AP/Cl): 413.2 (M+H)+.

### Example 66

### N-{5-[cis-3-(2-Methoxy-phenyl)-cyclobutyl]-2H-pyrazol-3-yl}-2-pyridin-3-yl-acetamide

1H NMR (400 MHz, CD3OD): δ 8.95 (s, 1H), 8.82 (d, J=5.8 Hz, 1H), 8.67 (d, J=8.3 Hz, 1H), 8.11 (m, 1H), 7.17 (m, 2H), 6.89 (m, 2H), 6.32 (s, 1H), 4.21 (s, 2H), 3.81 (s, 3H), 3.76 (m, 1H), 3.62 (m, 1H), 2.82 (m, 2H), 2.34 (m, 2H); MS (AP/Cl): 363.2.

### Example 67

### N-{5-[cis-3-(2-Methoxy-phenyl)-cyclobutyl]-2H-pyrazol-3-yl}-2-naphthalen-1-yl-acetamide hydrochloride

1H NMR (400 MHz, CD3OD): δ 8.03 (d, J=7.9 Hz, 1H), 7.89 (d, J=7.5 Hz, 1H), 7.83 (d, J=7.5 Hz, 1H), 7.5 (m, 4H), 7.17 (m, 2H), 6.91 (m, 2H), 6.22 (s, 1H), 4.27 (s, 2H), 3.80 (s, 3H), 3.75 (m, 1H), 3.60 (m, 1H), 2.81 (m, 2H), 2.32 (m, 2H); MS (AP/Cl): 412.2.

### Example 68

### N-{5-[cis-3-(4-Methoxy-phenyl)-cyclobutyl]-2H-pyrazol-3-yl}-2-naphthalen-1-yl-acetamide

1H NMR (400 MHz, CDCl₃): δ 7.97 (m, 2H), 7.83 (d, J=7.5 Hz, 1H), 7.77 (m, 1H), 7.49 (m, 2H), 7.40 (m, 2H), 7.11 (d, J=8.3 Hz, 2H), 6.84 (d, J=8.7 Hz, 2H), 6.51 (s, 1H), 4.08 (s, 2H), 3.78 (s, 3H), 3.4 (m, 2H), 2.69 (m, 2H), 2.18 (m, 2H); MS (AP/Cl): 412.2; minor isomer, characteristic 1H NMR signals: 6.65 (s), 2.51 (m).

### Example 69

### N-{5-[cis-3-(4-Chloro-phenyl)-cyclobutyl]-2H-pyrazol-3-yl}-2-naphthalen-1-yl-acetamide

1H NMR (400 MHz, CDCl₃): d 7.96 (d, J=7.5 Hz, 1H), 7.85 (m, 2H), 7.74 (s, 1H), 7.51 (m, 2H), 7.42 (m, 2H), 7.26 (m, 1H), 7.12 (d, J=8.3 Hz, 2H), 6.49 (s, 1H), 4.12 (s, 2H), 3.40 (m, 2H), 2.72 (m, 2H), 2.20 (m, 2H); MS (AP/Cl): 416.1, 418.1 (M+H)+; minor isomer, characteristic 1H NMR signals: δ 6.65 (s), 2.55 (m).

### Example 70

### 2-Naphthalen-1-yl-N-[5-(cis-3-p-tolyl-cyclobutyl)-2H-pyrazol-3-yl]-acetamide

1H NMR (400 MHz, CDCl₃): d 8.04 (s, 1H), 7.95 (d, J=7.9 Hz, 1H), 7.81 (d, J=8.3 Hz, 1H), 7.75 (m, 1H), 7.47 (m, 2H), 7.36 (m, 2H), 7.10 (m, 3H), 6.52 (s, 1H), 4.06 (s, 2H), 3.38 (m, 2H), 2.70 (m, 2H), 2.32 (s, 3H), 2.20 (m, 2H); MS (AP/Cl): 396.2; minor isomer, characteristic 1H NMR signals: δ 6.65 (s), 3.65 (m), 2.51 (m).

### Example 71

### 2-(4-Methoxy-phenyl)-N-{5-[cis-3-(2-methoxy-phenyl)-cyclobutyl]-2H-pyrazol-3-yl}-acetamide

1H NMR (400 MHz, CD3OD): δ 7.23 (d, J=8.7 Hz, 2H), 7.16 (d, J=7.5 Hz, 2H), 6.89 (m, 4H), 6.21 (s, 1H), 3.80 (s, 3H), 3.76 (s, 3H), 3.7 (m, 1H), 3.67 (s, 2H), 3.59 (m, 1H), 2.79 (m, 2H), 2.29 (m, 2H); MS (AP/Cl): 392.2 (M+H)+.

The following additional Examples of compounds of the invention were synthesized as described herein:

N-{5-[cis-3-(4-Hydroxy-phenyl)-cyclobutyl]-1H-pyrazol-3-yl}-2-quinolin-6-yl-acetamide;

N-{5-[cis-3-(3-Hydroxy-phenyl)-cyclobutyl]-1H-pyrazol-3-yl}-2-quinolin-6-yl-acetamide;

2-Naphthalen-1-yl-N-[5-(cis-3-pyridin-3-yl-cyclobutyl)-2H-pyrazol-3-yl]-acetamide;

N-[5-(cis-3-Naphthalen-2-yl-cyclobutyl)-2H-pyrazol-3-yl]-2-pyridin-3-yl-acetamide;

N-(5-Indan-2-yl-1H-pyrazol-3-yl)-2-quinolin-6-yl-acetamide;

N-[5-(cis-3-Pyridin-2-yl-cyclobutyl)-2H-pyrazol-3-yl]-2-quinolin-6-yl-acetamide;

N-[5-(cis-3-Pyridin-2-yl-cyclobutyl)-2H-pyrazol-3-yl]-2-quinolin-6-yl-acetamide;

2-(4-Methoxy-phenyl)-N-[5-(cis-3-pyridin-4-yl-cyclobutyl)-2H-pyrazol-3-yl]-acetamide;

N-{5-[3-(cis-2-Dimethylaminomethyl-phenyl)-cyclobutyl]-2H-pyrazol-3-yl}-2-(4-methoxy-phenyl)-acetamide;

N-(5-{cis-3-[3-(2-Dimethylamino-ethoxy)-phenyl]-cyclobutyl}-2H-pyrazol-3-yl)-2-(4-methoxy-phenyl)-acetamide;

N-{5-[cis-3-(2-Hydroxy-phenyl)-cyclobutyl]-2H-pyrazol-3-yl}-2-(4-methoxy-phenyl)-acetamide;

N-(5-{cis-3-[2-(2-Dimethylamino-ethoxy)-phenyl]-cyclobutyl}-2H-pyrazol-3-yl)-2-(4-methoxy-phenyl)-acetamide;

2-(4-Methoxy-phenyl)-N-[5-(cis-3-phenyl-cyclobutyl)-2H-pyrazol-3-yl]-acetamide;

N-{5-[cis-3-(2-Fluoro-phenyl)-cyclobutyl]-2H-pyrazol-3-yl}-2-(4-methoxy-phenyl)-acetamide;

N-(5-{cis-3-[4-(Azetidin-3-yloxy)-phenyl]-cyclobutyl}-2H-pyrazol-3-yl)-2-(4-methoxy-phenyl)-acetamide;

N-(5-{cis-3-[2-(Azetidin-3-yloxy)-phenyl]-cyclobutyl}-2H-pyrazol-3-yl)-2-(4-methoxy-phenyl)-acetamide;

2-(4-Methoxy-phenyl)-N-{5-[cis-3-(2-methylsulfanyl-phenyl)-cyclobutyl]-2H-pyrazol-3-yl}-acetamide;

N-{5-[cis-3-(2-Amino-phenyl)-cyclobutyl]-2H-pyrazol-3-yl}-2-(4-methoxy-phenyl)-acetamide;

N-{5-[cis-3-(4-Cyano-phenyl)-cyclobutyl]-2 H-pyrazol-3-yl}-2-(4-methoxy-phenyl)-acetamide;

N-{5-[cis-3-(2-Cyano-phenyl)-3-hydroxy-cyclobutyl]-2H-pyrazol-3-yl}-2-(4-methoxy-phenyl)-acetamide;

N-{5-[cis-3-(2-Hydroxy-ethyl)-cyclobutyl]-1H-pyrazol-3-yl}-2-naphthalen-1-yl-acetamide;

N-{5-[cis-3-(3-Cyano-phenyl)-cyclobutyl]-2H-pyrazol-3-yl}-2-(4-methoxy-phenyl)-acetamide;

N-{5-[cis-3-(2-Cyano-phenyl)-cyclobutyl]-2H-pyrazol-3-yl}-2-(4-methoxy-phenyl)-acetamide;

N-{5-[cis-3-(3-Amino-phenyl)-cyclobutyl]-2H-pyrazol-3-yl}-2-(4-methoxy-phenyl)-acetamide;

4-(cis-3-{5-[2-(4-Methoxy-phenyl)-acetylamino]-1H-pyrazol-3-yl}-cyclobutyl)-benzoic acid methyl ester;

N-{5-[cis-3-(4-Hydroxymethyl-phenyl)-cyclobutyl]-2H-pyrazol-3-yl}-2-(4-methoxy-phenyl)-acetamide;

N-{5-[cis-3-(2-Hydroxy-phenyl)-cyclobutyl]-1H-pyrazol-3-yl}-2-phenyl-acetamide;

N-{5-[cis-3-(2-Hydroxy-phenyl)-cyclobutyl]-1H-pyrazol-3-yl}-2-quinolin-6-yl-acetamide;

N-{5-[cis-3-(2-Hydroxy-phenyl)-cyclobutyl]-1H-pyrazol-3-yl}-acetamide;

Cyclopropanecarboxylic acid {5-[cis-3-(2-hydroxy-phenyl)-cyclobutyl]-1H-pyrazol-3-yl}-amide;

N-{5-[cis-3-(2-Hydroxy-phenyl)-cyclobutyl]-1H-pyrazol-3-yl}-isobutyramide;

N-{5-[cis-3-(3-Aminomethyl-phenyl)-cyclobutyl]-2H-pyrazol-3-yl}-2-(4-methoxy-phenyl)-acetamide;

N-{5-[cis-3-(3-Dimethylaminomethyl-phenyl)-cyclobutyl]-2 H-pyrazol-3-yl}-2-(4-methoxy-phenyl)-acetamide;

3-(cis-3-{5-[2-(4-Methoxy-phenyl)-acetylamino]-1H-pyrazol-3-yl}-cyclobutyl)-benzoic acid methyl ester;

N-{5-[cis-3-(3-Hydroxymethyl-phenyl)-cyclobutyl]-2H-pyrazol-3-yl}-2-(4-methoxy-phenyl)-acetamide;

N-(5-{cis-3-[3-(1-Hydroxy-1-methyl-ethyl)-phenyl]-cyclobutyl}-2H-pyrazol-3-yl)-2-(4-methoxy-phenyl)-acetamide;

N-{5-[cis-3-(3-Ethylaminomethyl-phenyl)-cyclobutyl]-2H-pyrazol-3-yl}-2-(4-methoxy-phenyl)-acetamide;

N-{5-[cis-3-(3-Cyclobutylaminomethyl-phenyl)-cyclobutyl]-2H-pyrazol-3-yl}-2-(4-methoxy-phenyl)-acetamide;

2-(4-Methoxy-phenyl)-N-{5-[cis-3-(3-propylaminomethyl-phenyl)-cyclobutyl]-2H-pyrazol-3-yl}-acetamide;

N-{5-[cis-3-(3-Cyclopentylaminomethyl-phenyl)-cyclobutyl]-2H-pyrazol-3-yl}-2-(4-methoxy-phenyl)-acetamide;

N-(5-{cis-3-[3-(Benzylamino-methyl)-phenyl]-cyclobutyl}-2H-pyrazol-3-yl)-2-(4-methoxy-phenyl)-acetamide;

2-(4-Methoxy-phenyl)-N-{5-[3-(3-methylaminomethyl-phenyl)-cyclobutyl]-2H-pyrazol-3-yl}-acetamide;

N-{5-[cis-3-(3-Cyclopropylaminomethyl-phenyl)-cyclobutyl]-2H-pyrazol-3-yl}-2-(4-methoxy-phenyl)-acetamide;

2-(4-Methoxy-phenyl)-N-{5-[cis-3-(3-pyrrolidin-1-ylmethyl-phenyl)-cyclobutyl]-2H-pyrazol-3-yl}-acetamide;

N-{5-[cis-3-(3-Diethylaminomethyl-phenyl)-cyclobutyl]-2H-pyrazol-3-yl}-2-(4-methoxy-phenyl)-acetamide; and

N-{5-[cis-3-(3-Azetidin-1-ylmethyl-phenyl)-cyclobutyl]-2H-pyrazol-3-yl}-2-(4-methoxy-phenyl)-acetamide.

What is claimed is:

1. A compound of the formula 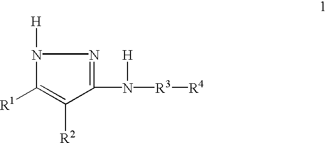
wherein R¹ is a straight chain or branched (C₁-C₈)alkyl, a straight chain or branched (C₂-C₈)alkenyl, a straight chain or branched (C₂-C₈)alkynyl, (C₃-C₈)cycloalkyl, (C₄-C₈)cycloalkenyl, (3-8 membered) heterocycloalkyl, (C₅-C₁₁)bicycloalkyl, (C₇-C₁₁)bicycloalkenyl, or (5-11 membered) heterobicycloalkyl, and wherein R¹ is optionally substituted with from one to six substituents R⁵ independently selected from F, Cl, Br, I, nitro, cyano, —CF₃, —NR⁷R⁸, —NR⁷C(═O)R⁸, —NR⁷C(═O)OR⁸, —NR⁷C(═O)NR⁸R⁹, —NR⁷S(═O)₂R⁸, —NR⁷S(═O)₂NR⁸R⁹, —OR⁷, —OC(═O)R⁷, —OC(═O)OR⁷, —C(═O)OR⁷, —C(═O)R⁷, —C(═O)NR⁷R⁸, —OC(═O)NR⁷R⁸, —OC(═O)SR⁷, —SR⁷, —S(═O)R⁷, —S(═O)₂R⁷, —S(═O)₂NR⁷R⁸, and R⁷;
R² is H, F, —CH₃, —CN, or —C(═O)OR⁷;
R³ is —C(═O)NR⁹—, —C(═O)O—, —C(═O)(CR¹⁰R¹¹)ₙ—, or —(CR¹⁰OR¹¹)ₙ—;
R⁴ is a straight chain or a branched (C₁-C₈)alkyl, a straight chain or a branched (C₂-C₈)alkenyl, a straight chain or branched (C₂-C₈ alkynyl), (C₃-C₈)cycloalkyl, (C₄-C₈)cycloalkenyl (3-8 membered) heterocycloalkyl, (C₅-C₁₁)bicycloalkyl, (C₇-C₁₁)bicycloalkenyl, (5-11 membered) heterobicycloalkyl, (C₆-C₁₄)aryl, or (5-14 membered) heteroaryl; and wherein R⁴ is optionally substituted with from one to three substitutents R⁶ independently selected from F, Cl, Br, I, nitro, cyano, —CF₃, —NR⁷R⁸, —NR⁷C(═O)R⁸, —NR⁷C(═O)OR⁸, —NR⁷C(═O)NR⁸R⁹, —NR⁷S(═O)₂R⁸, —NR⁷S(═O)₂NR⁸R⁹, —OR⁷, —OC(═O)R⁷, —OC(═O)OR⁷, —C(═O)OR⁷, —C(═O)R⁷, —C(═O)NR⁷R⁸, —OC(═O)NR⁷R⁸, —OC(═O)SR⁷, —SR⁷, —S(═O)R⁷, —S(═O)₂R⁷, —S(═O)₂NR⁷R⁸, or R⁷;
each R⁷, R⁸, and R⁹ is independently selected from H, straight chain or branched (C₁-C₈)alkyl, straight chain or branched (C₂-C₈)alkenyl, straight chain or branched (C₂-C₈ alkynyl), (C₃-C₈)cycloalkyl, (C₄-C₈)cycloalkenyl, (3-8 membered) heterocycloalkyl, (C₅-C₁₁)bicycloalkyl, (C₇-C₁₁)bicycloalkenyl, (5-11 membered) heterobicycloalkyl, (C₆-C₁₄)aryl, and (5-14 membered) heteroaryl, wherein R⁷, R⁸, and R⁹ are each independently optionally substituted with from one to six substituents independently selected from F, Cl, Br, I, —NO₂, —CN, —CF₃, —NR¹⁰R¹¹, —NR¹⁰C(═O)R¹¹, —NR¹⁰C(═O)OR¹¹, —NR¹⁰C(═O)NR¹¹R¹², —NR¹⁰S(═O)₂R¹¹, —NR¹⁰S(═O)₂NR¹¹R¹², —OR¹⁰, —OC(═O)R¹⁰, —OC(═O)OR¹¹, —OC(═O)NR¹⁰R¹¹, —OC(═O)SR¹⁰, —SR¹⁰, —S(═O)R¹⁰, —S(═O)₂R¹⁰, —S(═O)₂NR¹⁰R¹¹, —C(═O)R¹⁰, —C(═O)OR¹⁰, —C(═O)NR¹⁰R¹¹, and R¹⁰;
or, when R⁷ and R⁸ are as in NR⁷R⁸, they may instead optionally be connected to form with the nitrogen of NR⁷R⁸ to which they are attached a heterocycloalkyl moiety of from three to seven ring members, said heterocycloalkyl moiety optionally comprising one or two further heteroatoms independently selected from N, O, and S;
each R¹⁰, R¹¹, and R¹² is independently selected from H, straight chain or branched (C₁-C₈)alkyl, straight chain or branched (C₂-C₈)alkenyl, straight chain or branched (C₂-C₈ alkynyl), (C₃-C₈)cycloalkyl, (C₄-C₈)cycloalkenyl, (3-8 membered) heterocycloalkyl, (C₅-C₁₁)bicycloalkyl, (C₇-C₁₁)bicycloalkenyl, (5-11 membered) heterobicycloalkyl, (C₆-C₁₄)aryl, and (5-14 membered heteroaryl, wherein R¹⁰, R¹¹, and R¹² are each independently optionally substituted with from one to six substituents independently selected from F, Cl, Br, I, NO₂, —CN, —CF₃, —NR¹³R¹⁴, —NR¹³C(═O)R¹⁴, —NR¹³C(═O)OR¹⁴, —NR¹³C(═O)NR¹⁴R¹⁵, —NR¹³S(═O)₂R¹⁴, —NR¹³S(═O)₂NR¹⁴R¹⁵, —OR¹³, —OC(═O)R¹³, —OC(═O)OR¹³, —OC(═O)NR¹³R¹⁴, —OC(═O)SR¹³, —SR¹³, —S(═O)R¹³, —S(═O)₂R¹³, —S(═O)₂NR¹³R¹⁴, —C(═O)R¹³, —C(═O)OR¹³, —C(═O)NR¹³R¹⁴, and R¹³;
each R¹³, R¹⁴, and R¹⁵ is independently selected from H, straight chain or branched (C₁-C₈)alkyl, straight chain or branched (C₂-C₈)alkenyl, straight chain or branched (C₂-C₈ alkynyl), (C₃-C₈)cycloalkyl, (C₄-C₈)cycloalkenyl, (3-8 membered) heterocycloalkyl, (C₅-C₁₁)bicycloalkyl, (C₇-C₁₁)bicycloalkenyl, (5-11 membered) heterobicycloalkyl, (C₆-C₁₄)aryl, and (5-14 membered) heteroaryl, wherein R¹³, R¹⁴, and R¹⁵ are each independently optionally substituted with from one to six substituents independently selected from F, Cl, Br, I, NO₂, —CN, —CF₃, —NR¹⁶R¹⁷, —NR¹⁶C(═O)R¹⁷, —NR¹⁶C(═O)OR¹⁷, —NR¹⁶C(═O)NR¹⁷R¹⁸, —NR¹⁶S(═O)₂R¹⁷, —NR¹⁶S(═O)₂NR¹⁷R¹⁸ —OR¹⁶, —OC(═O)R¹⁶, —OC(═O)OR¹⁶, —OC(═O)NR¹⁶R¹⁷, —OC(═O)SR¹⁶, —SR¹⁶, —S(═O)R¹⁶, —S(═O)₂R¹⁶, —S(═O)₂NR¹⁶R¹⁷, —C(═O)R¹⁶, —C(═O)OR¹⁶, —C(═O)NR¹⁶R¹⁷, and R¹⁶
each R¹⁶, R¹⁷, and R¹⁸ is independently selected from H, straight chain or branched (C₁-C₈)alkyl, straight chain or branched (C₂-C₈)alkenyl, straight chain or branched (C₂-C₈ alkynyl), (C₃-C₈)cycloalkyl, (C₄-C₈)cycloalkenyl, (3-8 membered) heterocycloalkyl, (C₅-C₁₁)bicycloalkyl, (C₇-C₁₁)bicycloalkenyl, (5-11 membered) heterobicycloalkyl, (C₆-C₁₄)aryl, and (5-14 membered heteroaryl;
n is 0, 1, 2, or 3;
wherein R¹⁰ and R¹¹ in —C(═O)(CR¹⁰R¹¹)ₙ— and —(CR¹⁰R¹¹)ₙ— are for each iteration of n defined independently as recited above;
or a pharmaceutically acceptable salt thereof.

2. A compound according to claim 1, wherein R³ is —(CR¹⁰R¹¹)ₙ—, —C(═O)NH— or —C(═O)(CR¹⁰R¹¹)ₙ—.

3. A compound according to claim 2, wherein R³ is —(CR¹⁰OR¹¹)ₙ— and n is zero.

4. A compound according to claim 1, wherein R¹ is optionally substituted (C₃-C₈)cycloalkyl or optionally substituted (C₅-C₁₁) bicycloalkyl.

5. A compound according to claim 4, wherein R¹ is cyclopropyl, cyclobutyl, cyclopentyl, cyclohexyl, or norbornyl, each optionally substituted.

6. A compound according to claim 5, wherein R¹ is optionally substituted with from one to three substituents independently selected from F, Cl, Br, I, nitro, cyano, —CF₃, —NR⁷R⁸, —NR⁷C(═O)R⁸, —OR⁷, —C(═O)OR⁷, —C(═O)R⁷, and R⁷.

7. A compound according to claim 4, wherein R¹ is substituted with —NR⁷C(═O)R⁸, (C₆-C₁₄)aryl, (3-8 membered) heterocycloalkyl, or (5-14 membered) heteroaryl, and wherein said aryl, heterocycloalkyl, and heteroaryl are each optionally substituted with from one to six substituents independently selected from F, Cl, Br, I, —NO₂, —CN, —CF₃, —NR¹⁰R¹¹, —NR¹⁰C(═O)R¹¹, —NR¹⁰C(═O)OR¹¹, —NR¹⁰C(═O)NR¹¹R¹², —NR¹⁰S(═O)₂R¹¹, —NR¹⁰S(═O)₂NR¹¹R¹², —OR¹⁰, —OC(═O)R¹⁰, —OC(═O)OR¹⁰, —OC(═O)NR¹⁰R¹¹, —OC(═O)SR¹⁰, —SR¹⁰, —S(═O)R¹⁰, —S(═O)₂R¹⁰, —S(═O)₂NR¹⁰OR¹¹, —C(═O)R¹⁰, —C(═O)OR¹⁰, —C(═O)NR¹⁰R¹¹, and R¹⁰.

8. A compound according to claim 4, wherein R¹ is optionally substituted bicyclo-[3.1.0]-hexyl.

9. A compound according to claim 1, wherein R¹ is optionally substituted straight chain or branched (C₁-C₈)alkyl or optionally substituted straight chain or branched (C₂-C₈)alkenyl.

10. A compound according to claim 1, wherein R⁴ is (C₆-C₁₄)aryl or (5-14 membered) heteroaryl, each optionally substituted.

11. A compound according to claim 10, wherein R⁴ is optionally substituted phenyl or optionally substituted pyridyl.

12. A compound according to claim 10, wherein R⁴ is naphthyl, quinolyl, isoquinolyl, pyrimidinyl, pyrazinyl, or pyridazyl, each optionally substituted.

13. A compound according to claim 12, wherein R⁴ is unsubstituted.

14. A compound according to claim 1 wherein R² is hydrogen.

15. A compound of claim 1, selected from the group consisting of:
(5-ethyl-2H-pyrazol-3-yl)-(6-methoxy-pyridin-2-yl)-amine;
(5-cyclobutyl-2H-pyrazol-3-yl)-(6-methoxy-pyridin-2-yl)-amine
(5-cyclobutyl-2H-pyrazol-3-yl)-naphthalen-2-yl-amine;
(5-cyclobutyl-2H-pyrazol-3-yl)-naphthalen-1-yl-amine;
N-(5-cyclobutyl-2H-pyrazol-3-yl)-N′,N′-dimethyl-naphthalene-1,4-diamine;
N-(5-cyclobutyl-2H-pyrazol-3-yl)-N′,N′-dimethyl-pyridine-2,6-diamine;
(5-cyclobutyl-2H-pyrazol-3-yl)-(6-trifluoromethyl-pyridin-2-yl)-amine;
(3-benzyloxy-phenyl)-(5-cyclobutyl-2H-pyrazol-3-yl)-amine;
(5-cyclobutyl-2H-pyrazol-3-yl)-(3-trifluoromethyl-phenyl)-amine;
N-(5-cyclobutyl-2H-pyrazol-3-yl)-N′,N′-dimethyl-benzene-1,3-diamine;
(5-cyclobutyl-2H-pyrazol-3-yl)-(3-methoxy-phenyl)-amine;
(5-cyclobutyl-2H-pyrazol-3-yl)-(4-nitro-phenyl)-amine;
(4-chloro-benzyl)-(5-cyclobutyl-2H-pyrazol-3-yl)-amine;
(3-bromo-phenyl)-(5-cyclobutyl-2H-pyrazol-3-yl)-amine;
(5-cyclobutyl-2H-pyrazol-3-yl)-quinolin-2-yl-amine;
[5-(1,4-dioxa-spiro[4.4]non-7-yl)-1H-pyrazol-3-yl]-(3-trifluoromethyl-phenyl)-amine;
(6-chloro-pyridin-2-yl)-(5-cyclobutyl-2H-pyrazol-3-yl)-amine;
3-[5-(3-trifluoromethyl-phenylamino)-2H-pyrazol-3-yl]-cyclopentanone;
(5-cyclobutyl-2H-pyrazol-3-yl)-(6-methoxy-4-methyl-quinolin-2-yl)-amine;
(5-cyclobutyl-2H-pyrazol-3-yl)-(3-trifluoromethoxy-phenyl)-amine;
(2-chloro-4-nitro-phenyl)-(5-cyclobutyl-2H-pyrazol-3-yl)-amine;
3-trans-[5-(3-trifluoromethyl-phenylamino)-2H-pyrazol-3-yl]-cyclopentanone;
(3,5-bis-trifluoromethyl-phenyl)-(5-cyclobutyl-2H-pyrazol-3-yl)-amine;
[5-(3-cis-benzylamino-cyclopentyl)-1H-pyrazol-3-yl]-(3-trifluoromethyl-phenyl)-amine;
{5-[3-cis-(4-methoxy-benzylamino)-cyclopentyl]-1H-pyrazol-3-yl}-(3-trifluoromethyl-phenyl)-amine;
4-(5-cyclobutyl-2H-pyrazol-3-ylamino)-benzonitrile;
(5-cyclobutyl-2H-pyrazol-3-yl)-(3-fluoro-phenyl)-amine;
(5-cyclobutyl-2H-pyrazol-3-yl)-(3,5-dichloro-phenyl)-amine;
(2-bromo-phenyl)-(5-cyclobutyl-2H-pyrazol-3-yl)-amine;
N-{cis-3-[5-(3-trifluoromethyl-phenylamino)-2H-pyrazol-3-yl]-cyclopentyl}-acetamide;
pyridin-2-yl-{3-trans-[5-(3-trifluoromethyl-phenylamino)-2H-pyrazol-3-yl]-cyclopentyl}-amine;
(5-cyclobutyl-1H-pyrazol-3-yl)-(4-methoxy-phenyl)-amine;
pyridine-2-carboxylic acid {3-[5-(3-trifluoromethyl-phenylamino)-2H-pyrazol-3-yl]-cyclopentyl}-amide;
3-trifluoromethyl-N-{3-[5-(3-trifluoromethyl-phenylamino)-2H-pyrazol-3-yl]-cyclopentyl}-benzamide;
cyclobutanecarboxylic acid {3-[5-(3-trifluoromethyl-phenylamino)-2H-pyrazol-3-yl]-cyclopentyl}-amide;
2,2-dimethyl-N-{3-[5-(3-trifluoromethyl-phenylamino)-2H-pyrazol-3-yl]-cyclopentyl}-propionamide;
4-fluoro-N-{3-[5-(3-trifluoromethyl-phenylamino)-2H-pyrazol-3-yl]-cyclopentyl}-benzamide;
2,2,2-trifluoro-N-{3-[5-(3-trifluoromethyl-phenylamino)-2H-pyrazol-3-yl]-cyclopentyl}-acetamide;
cyclopropanecarboxylic acid {3-[5-(3-trifluoromethyl-phenylamino)-2H-pyrazol-3-yl]-cyclopentyl}-amide;
N-{3-[5-(3-trifluoromethyl-phenylamino)-2H-pyrazol-3-yl]-cyclopentyl}-propionamide;
cyclohexanecarboxylic acid {3-[5-(3-trifluoromethyl-phenylamino)-2H-pyrazol-3-yl]-cyclopentyl}-amide;
N-[5-(3-acetylamino-cyclopentyl)-2H-pyrazol-3-yl]-2-naphthalen-1-yl-acetamide;
cyclopropanecarboxylic acid {3-[5-(2-naphthalen-1-yl-acetylamino)-1H-pyrazol-3-yl]-cyclopentyl}-amide;
2-naphthalen-1-yl-N-{5-[3-(2,2,2-trifluoro-acetylamino)-cyclopentyl]-2H-pyrazol-3-yl}-acetamide;
N-{3-[5-(2-naphthalen-1-yl-acetylamino)-1H-pyrazol-3-yl]-cyclopentyl}-benzamide;
N-(5-hydroxymethyl-1H-pyrazol-3-yl)-2-naphthalen-1-yl-acetamide;
2-naphthalen-1-yl-N-[5-(thiazol-2-ylaminomethyl)-1H-pyrazol-3-yl]-acetamide;
N-[5-((1S)-hydroxy-ethyl)-2H-pyrazol-3-yl]-2-naphthalen-1-yl-acetamide;
N-{5-[(1S)-(benzooxazol-2-yloxy)-ethyl]-1H-pyrazol-3-yl}-2-naphthalen-1-yl-acetamide;
N-{5-[(1S)-(benzothiazol-2-yloxy)-ethyl]-1H-pyrazol-3-yl}-2-naphthalen-1-yl-acetamide;
N-[5-(3-hydroxy-1-methyl-propyl)-1H-pyrazol-3-yl]-2-naphthalen-1-yl-acetamide;
N-[5-(benzothiazol-2-yloxymethyl)-1H-pyrazol-3-yl]-2-naphthalen-1-yl-acetamide;
N-{5-[3-(benzothiazol-2-yloxy)-1-methyl-propyl]-1H-pyrazol-3-yl}-2-naphthalen-1-yl-acetamide;
N-[5-(2-hydroxy-(1S)-methyl-ethyl)-2H-pyrazol-3-yl]-2-naphthalen-1-yl-acetamide;
N-{5-[(1R)-(benzothiazol-2-yloxy)-ethyl]-1H-pyrazol-3-yl}-2-naphthalen-1-yl-acetamide;
N-[5-(3-acetylamino-1-methyl-propyl)-1H-pyrazol-3-yl]-2-naphthalen-1-yl-acetamide;
3-methoxy-N-{cis-3-[5-(2-naphthalen-1-yl-acetylamino)-2H-pyrazol-3-yl]-cyclobutyl}-benzamide;
N-[5-(cis-3-acetylamino-cyclobutyl)-1H-pyrazol-3-yl]-2-naphthalen-1-yl-acetamide;
N-{cis-3-[5-(2-naphthalen-1-yl-acetylamino)-2H-pyrazol-3-yl]-cyclobutyl}-benzamide;
2-cyclopropyl-N-{cis-3-[5-(2-naphthalen-1-yl-acetylamino)-2H-pyrazol-3-yl]-cyclobutyl}-acetamide;
6-chloro-pyridine-2-carboxylic acid {cis-3-[5-(2-naphthalen-1-yl-acetylamino)-2H-pyrazol-3-yl]-cyclobutyl}-amide;
quinoline-2-carboxylic acid {cis-3-[5-(2-naphthalen-1-yl-acetylamino)-2H-pyrazol-3-yl]-cyclobutyl}-amide;
pyrazine-2-carboxylic acid {cis-3-[5-(2-naphthalen-1-yl-acetylamino)-2H-pyrazol-3-yl]-cyclobutyl}-amide;
4-methoxy-N-{cis-3-[5-(2-naphthalen-1-yl-acetylamino)-2H-pyrazol-3-yl]-cyclobutyl}-benzamide;
N-{cis-3-[5-(2-naphthalen-1-yl-acetylamino)-2H-pyrazol-3-yl]-cyclobutyl}-3-nitro-benzamide;
N-{cis-3-[5-(2-naphthalen-1-yl-acetylamino)-2H-pyrazol-3-yl]-cyclobutyl}-3-trifluoromethyl-benzamide;
N-{cis-3-[5-(2-naphthalen-1-yl-acetylamino)-2H-pyrazol-3-yl]-cyclobutyl}-isobutyramide;
2-phenyl-cyclopropanecarboxylic acid {cis-3-[5-(2-naphthalen-1-yl-acetylamino)-2H-pyrazol-3-yl]-cyclobutyl}-amide;
N-{5-[cis-3-(benzooxazol-2-yloxy)-cyclobutyl]-1H-pyrazol-3-yl}-2-naphthalen-1-yl-acetamide;
4-dimethylamino-N-{cis-3-[5-(2-naphthalen-1-yl-acetylamino)-2H-pyrazol-3-yl]--cyclobutyl}-benzamide;
3,5-dimethoxy-N-{cis-3-[5-(2-naphthalen-1-yl-acetylamino)-2H-pyrazol-3-yl]-cyclobutyl}-benzamide;
2-naphthalen-1-yl-N-[5-(cis-3-phenyl-cyclobutyl)-2H-pyrazol-3-yl]-acetamide;
N-{5-[cis-3-(3-methoxy-phenyl)-cyclobutyl]-2H-pyrazol-3-yl}-2-naphthalen-1-yl-acetamide;
N-{5-[cis-3-(2-methoxy-phenyl)-cyclobutyl]-2H-pyrazol-3-yl}-2-naphthalen-1-yl-acetamide;
N-{5-[cis-3-(4-methoxy-phenyl)-cyclobutyl]-2H-pyrazol-3-yl}-2-naphthalen-1-yl-acetamide;
2-naphthalen-1-yl-N-[5-(cis-3-p-tolyl-cyclobutyl)-2H-pyrazol-3-yl]-acetamide;
N-{5-[cis-3-(4-chloro-phenyl)-cyclobutyl]-2H-pyrazol-3-yl}-2-naphthalen-1-yl-acetamide;
2-(4-methoxy-phenyl)-N-{5-[cis-3-(2-methoxy-phenyl)-cyclobutyl]-2H-pyrazol-3-yl}-acetamide;
N-{5-[cis-3-(2-methoxy-phenyl)-cyclobutyl]-2H-pyrazol-3-yl}-2-quinolin-6-yl-acetamide;
N-{5-[cis-3-(2-methoxy-phenyl)-cyclobutyl]-2H-pyrazol-3-yl}-2-phenyl-acetamide;
N-{5-[cis-3-(2-methoxy-phenyl)-cyclobutyl]-2H-pyrazol-3-yl}-2-pyridin-3-yl-acetamide;
N-{5-[cis-3-(4-methoxy-phenyl)-cyclobutyl]-1H-pyrazol-3-yl}-2-quinolin-6-yl-acetamide;
2-quinolin-6-yl-N-[5-(cis-3-p-tolyl-cyclobutyl)-1H-pyrazol-3-yl]-acetamide;
N-{5-[cis-3-(4-fluoro-phenyl)-cyclobutyl]-1H-pyrazol-3-yl}-2-quinolin-6-yl-acetamide;
N-{5-[cis-3-(4-chloro-phenyl)-cyclobutyl]-1H-pyrazol-3-yl}-2-quinolin-6-yl-acetamide;
2-quinolin-6-yl-N-[5-(cis-3-m-tolyl-cyclobutyl)-1H-pyrazol-3-yl]-acetamide;
4-dimethylamino-N-{cis-3-[5-(2-naphthalen-1-yl-acetylamino)-2H-pyrazol-3-yl]-cyclobutyl}-benzamide;
2-naphthalen-1-yl-N-{5-[cis-3-(pyridin-2-yloxy)-cyclobutyl]-1H-pyrazol-3-yl}-acetamide;
6-methyl-pyridine-2-carboxylic acid {cis-3-[5-(2-naphthalen-1-yl-acetylamino)-2H-pyrazol-3-yl]-cyclobutyl}-amide;
2-phenyl-cyclopropanecarboxylic acid methyl-{cis-3-[5-(2-naphthalen-1-yl-acetylamino)-2H-pyrazol-3-yl]-cyclobutyl}-amide;
N-{5-[cis-3-(3-methyl-pyrazin-2-yloxy)-cyclobutyl]-1H-pyrazol-3-yl}-2-naphthalen-1-yl-acetamide;
{5-[cis-3-(2-methoxy-phenyl)-cyclobutyl]-1H-pyrazol-3-yl}-(6-methoxy-pyridin-2-yl)-amine;
N-{5-[cis-3-(3,6-dimethyl-pyrazin-2-yloxy)-cyclobutyl]-1H-pyrazol-3-yl}-2-naphthalen-1-yl-acetamide;
N-{5-[cis-3-(3-methoxy-pyridin-2-yloxy)-cyclobutyl]-1H-pyrazol-3-yl}-2-naphthalen-1-yl-acetamide;
2-methyl-cyclopropanecarboxylic acid {cis-3-[5-(2-naphthalen-1-yl-acetylamino)-2H-pyrazol-3-yl]-cyclobutyl}-amide;
2-naphthalen-1-yl-N-{5-[cis-3-(3-trifluoromethyl-pyridin-2-yloxy)-cyclobutyl]-1H-pyrazol-3-yl}-acetamide;
2-naphthalen-1-yl-N-{5-[cis-3-(3-nitro-pyridin-2-yloxy)-cyclobutyl]-1H-pyrazol-3-yl}-acetamide;
N-{5-[cis-3-(benzothiazol-2-yloxy)-cyclobutyl]-1H-pyrazol-3-yl}-2-naphthalen-1-yl-acetamide;
2-naphthalen-1-yl-N-{5-[cis-3-(4-trifluoromethyl-pyrimidin-2-yloxy)-cyclobutyl]-1H-pyrazol-3-yl}-acetamide;
2-naphthalen-1-yl-N-{5-[3-(5-nitro-pyridin-2-yloxy)-cyclobutyl]-1H-pyrazol-3-yl}-acetamide;
2-naphthalen-1-yl-N-{5-[3-(pyrimidin-2-yloxy)-cyclobutyl]-1H-pyrazol-3-yl}-acetamide;
2-naphthalen-1-yl-N-{5-[3-(5-trifluoromethyl-pyridin-2-yloxy)-cyclobutyl]-1H-pyrazol-3-yl}-acetamide;
N-{5-[3-(6-methoxy-pyridazin-3-yloxy)-cyclobutyl]-1H-pyrazol-3-yl}-2-naphthalen-1-acetamide;
2-naphthalen-1-yl-N-{5-[3-(pyrazin-2-yloxy)-cyclobutyl]-1H-pyrazol-3-yl}-acetamide;
N-{5-[3-(6-methyl-pyridin-2-yloxy)-cyclobutyl]-1H-pyrazol-3-yl}-2-naphthalen-1-acetamide;
N-{5-[3-(6-chloro-benzothiazol-2-yloxy)-cyclobutyl]-1H-pyrazol-3-yl}-2-naphthalen-1-yl-acetamide;
N-{5-[3-(6-methoxy-benzothiazol-2-yloxy)-cyclobutyl]-1H-pyrazol-3-yl}-2-naphthalen-1-yl-acetamide;
N-{5-[cis-3-(4-Hydroxy-phenyl)-cyclobutyl]-1H-pyrazol-3-yl}-2-quinolin-6-yl-acetamide
N-{5-[cis-3-(3-Hydroxy-phenyl)-cyclobutyl]-1H-pyrazol-3-yl}-2-quinolin-6-yl-acetamide
2-Naphthalen-1-yl-N-[5-(cis-3-pyridin-3-yl-cyclobutyl)-2H-pyrazol-3-yl]-acetamide;
N-[5-(cis-3-Naphthalen-2-yl-cyclobutyl)-2H-pyrazol-3-yl]-2-pyridin-3-yl-acetamide;
N-(5-Indan-2-yl-1H-pyrazol-3-yl)-2-quinolin-6-yl-acetamide;
N-[5-(cis-3-Pyridin-2-yl-cyclobutyl)-2H-pyrazol-3-yl]-2-quinolin-6-yl-acetamide;
N-[5-(cis-3-Pyridin-2-yl-cyclobutyl)-2H-pyrazol-3-yl]-2-quinolin-6-yl-acetamide;
2-(4-Methoxy-phenyl)-N-[5-(cis-3-pyridin-4-yl-cyclobutyl)-2H-pyrazol-3-yl]-acetamide;
N-{5-[3-(cis-2-Dimethylaminomethyl-phenyl)-cyclobutyl]-2H-pyrazol-3-yl}-2-(4-methoxy-phenyl)-acetamide;
N-(5-{cis-3-[3-(2-Dimethylamino-ethoxy)-phenyl]-cyclobutyl}-2H-pyrazol-3-yl)-2-(4-methoxy-phenyl)-acetamide;
N-{5-[cis-3-(2-Hydroxy-phenyl)-cyclobutyl]-2H-pyrazol-3-yl}-2-(4-methoxy-phenyl)-acetamide;
N-(5-{cis-3-[2-(2-Dimethylamino-ethoxy)-phenyl]-cyclobutyl}-2H-pyrazol-3-yl)-2-(4-methoxy-phenyl)-acetamide;
2-(4-Methoxy-phenyl)-N-[5-(cis-3-phenyl-cyclobutyl)-2H-pyrazol-3-yl]-acetamide;
N-{5-[cis-3-(2-Fluoro-phenyl)-cyclobutyl]-2H-pyrazol-3-yl}-2-(4-methoxy-phenyl)-acetamide;
N-(5-{cis-3-[4-(Azetidin-3-yloxy)-phenyl]-cyclobutyl}-2H-pyrazol-3-yl)-2-(4-methoxy-phenyl)-acetamide;
N-(5-{cis-3-[2-(Azetidin-3-yloxy)-phenyl]-cyclobutyl}-2H-pyrazol-3-yl)-2-(4-methoxy-phenyl)-acetamide;
2-(4-Methoxy-phenyl)-N-{5-[cis-3-(2-methylsulfanyl-phenyl)-cyclobutyl]-2H-pyrazol-3-yl}-acetamide;
N-{5-[cis-3-(2-Amino-phenyl)-cyclobutyl]-2H-pyrazol-3-yl}-2-(4-methoxy-phenyl)-acetamide;
N-{5-[cis-3-(4-Cyano-phenyl)-cyclobutyl]-2H-pyrazol-3-yl}-2-(4-methoxy-phenyl)-acetamide;
N-{5-[cis-3-(2-Cyano-phenyl)-3-hydroxy-cyclobutyl]-2H-pyrazol-3-yl}-2-(4-methoxy-phenyl)-acetamide;
N-{5-[cis-3-(2-Hydroxy-ethyl)-cyclobutyl]-1H-pyrazol-3-yl}-2-naphthalen-1-yl-acetamide;
N-{5-[cis-3-(3-Cyano-phenyl)-cyclobutyl]-2H-pyrazol-3-yl}-2-(4-methoxy-phenyl)-acetamide;
N-{5-[cis-3-(2-Cyano-phenyl)-cyclobutyl]-2H-pyrazol-3-yl}-2-(4-methoxy-phenyl)-acetamide;
N-{5-[cis-3-(3-Amino-phenyl)-cyclobutyl]-2H-pyrazol-3-yl}-2-(4-methoxy-phenyl)-acetamide;
4-(cis-3-{5-[2-(4-Methoxy-phenyl)-acetylamino]-1H-pyrazol-3-yl}-cyclobutyl)-benzoic acid methyl ester;
N-{5-[cis-3-(4-Hydroxymethyl-phenyl)-cyclobutyl]-2H-pyrazol-3-yl}-2-(4-methoxy-phenyl)-acetamide;
N-{5-[cis-3-(2-Hydroxy-phenyl)-cyclobutyl]-1H-pyrazol-3-yl}-2-phenyl-acetamide;
N-{5-[cis-3-(2-Hydroxy-phenyl)-cyclobutyl]-1H-pyrazol-3-yl}-2-quinolin-6-yl-acetamide;
N-{5-[cis-3-(2-Hydroxy-phenyl)-cyclobutyl]-1H-pyrazol-3-yl}-acetamide;
Cyclopropanecarboxylic acid {5-[cis-3-(2-hydroxy-phenyl)-cyclobutyl]-1H-pyrazol-3-yl}-amide;
N-{5-[cis-3-(2-Hydroxy-phenyl)-cyclobutyl]-1H-pyrazol-3-yl}-isobutyramide;
N-{5-[cis-3-(3-Aminomethyl-phenyl)-cyclobutyl]-2H-pyrazol-3-yl}-2-(4-methoxy-phenyl)-acetamide;
N-{5-[cis-3-(3-Dimethylaminomethyl-phenyl)-cyclobutyl]-2H-pyrazol-3-yl}-2-(4-methoxy-phenyl)-acetamide;
3-(cis-3-{5-[2-(4-Methoxy-phenyl)-acetylamino]-1H-pyrazol-3-yl}-cyclobutyl)-benzoic acid methyl ester;
N-{5-[cis-3-(3-Hydroxymethyl-phenyl)-cyclobutyl]-2H-pyrazol-3-yl}-2-(4-methoxy-phenyl)-acetamide;
N-(5-{cis-3-[3-(1-Hydroxy-1-methyl-ethyl)-phenyl]-cyclobutyl}-2H-pyrazol-3-yl)-2-(4-methoxy-phenyl)-acetamide;
N-{5-[cis-3-(3-Ethylaminomethyl-phenyl)-cyclobutyl]-2H-pyrazol-3-yl}-2-(4-methoxy-phenyl)-acetamide;
N-{5-[cis-3-(3-Cyclobutylaminomethyl-phenyl)-cyclobutyl]-2H-pyrazol-3-yl}-2-(4-methoxy-phenyl)-acetamide;
2-(4-Methoxy-phenyl)-N-{5-[cis-3-(3-propylaminomethyl-phenyl)-cyclobutyl]-2H-pyrazol-3-yl}-acetamide;
N-{5-[cis-3-(3-Cyclopentylaminomethyl-phenyl)-cyclobutyl]-2H-pyrazol-3-yl}-2-(4-methoxy-phenyl)-acetamide;
N-(5-{cis-3-[3-(Benzylamino-methyl)-phenyl]-cyclobutyl}-2H-pyrazol-3-yl)-2-(4-methoxy-phenyl)-acetamide;
2-(4-Methoxy-phenyl)-N-{5-[3-(3-methylaminomethyl-phenyl)-cyclobutyl]-2H-pyrazol-3-yl}-acetamide;
N-{5-[cis-3-(3-Cyclopropylaminomethyl-phenyl)-cyclobutyl]-2H-pyrazol-3-yl}-2-(4-methoxy-phenyl)-acetamide;
2-(4-Methoxy-phenyl)-N-{5-[cis-3-(3-pyrrolidin-1-ylmethyl-phenyl)-cyclobutyl]-2H-pyrazol-3-yl}-acetamide;
N-{5-[cis-3-(3-Diethylaminomethyl-phenyl)-cyclobutyl]-2H-pyrazol-3-yl}-2-(4-methoxy-phenyl)-acetamide;
N-{5-[cis-3-(3-Azetidin-1-ylmethyl-phenyl)-cyclobutyl]-2H-pyrazol-3-yl}-2-(4-methoxy-phenyl)-acetamide; and
pharmaceutically acceptable salts of the foregoing compounds.

16. A pharmaceutical composition for treating a) a disease or condition comprising abnormal cell growth; b) a neurodegenerative disease or condition; or c) a disease or condition the treatment of which can be effected or facilitated by inhibiting GSK-3, in a mammal comprising a compound of claim 1 in an amount effective intreating said disease or condition, and a pharmaceutically acceptable carrier.

17. A pharmaceutical composition for treating a disease or condition the treatment of which can be effected or facilitated by altering dopamine mediated neurotransmission in a mammal comprising a compound according to claim 1 in an amount effective in treating said disease or condition and a pharmaceutically acceptable carrier.

18. A pharmaceutical composition according to claim 17 wherein the disease or condition is selected from Parkinson's disease, schizophrenia, schizophreniform disorder, schizoaffective disorder, delusional disorder, substance-induced psychotic disorder, personality disorder of the paranoid type, personality disorder of the schizoid type, drug addiction, drug withdrawal, obsessive compulsive disorder, Tourette's syndrome, depression, a mood episode, post-stroke depression, major depressive disorder, dysthymic disorder, minor depressive disorder, premenstrual dysphoric disorder, post-psychotic depressive disorder of schizophrenia, a major depressive disorder superimposed on a psychotic disorder such as delusional disorder or schizophrenia, a bipolar disorder, anxiety; attention deficit and hyperactivity disorder; and attention deficit disorder.

19. A method for treating a disease or condition comprising abnormal cell growth in a mammal comprising administering to the mammal a compound of claim 1 in an amount effective in inhibiting abnormal cell growth.

20. A method according to claim 19, wherein the disease or condition comprising abnormal cell growth is cancer.

21. A method according to claim 19, for treating a disease or condition comprising abnormal cell growth in a mammal, wherein the disease or condition is selected from benign prostate hyperplasia, familial adenomatosis polyposis, neuro-fibromatosis, atherosclerosis, pulmonary fibrosis, arthritis, psoriasis, glomerulonephritis, restenosis, hypertrophic scar formation, inflammatory bowel disease, transplantation rejection, fungal infection, and endotoxic shock.

22. A method for treating a diseases or condition comprising abnormal cell growth in a mammal comprising administering to the mammal a compound of claim 1 in an amount effective to inhibit cdk2 activity.

23. A method according to claim 22, wherein the disease or condition comprising abnormal cell growth is cancer.

24. A method according to claim 22, for treating a disease or condition comprising abnormal cell growth in a mammal, wherein the disease or condition is selected from benign prostate hyperplasia, familial adenomatosis polyposis, neuro-fibromatosis, atherosclerosis, pulmonary fibrosis, arthritis, psoriasis, glomerulonephritis, restenosis, hypertrophic scar formation, inflammatory bowel disease, transplantation rejection, fungal infection, and endotoxic shock.

25. A method for treating a neurodegenerative disease or condition in a mammal comprising administering to the mammal a compound of claim 1 in an amount effective in treating said disease or condition.

26. A method according to claim 25 wherein the neurodegenerative disease or condition is selected from Huntington's disease, stroke, spinal cord trauma, traumatic brain injury, multiinfarct dementia, epilepsy, amyotrophic lateral sclerosis, pain, viral induced dementia for example AIDS induced dementia, neurodegeneration associated with bacterial infection, migraine, hypoglycemia, urinary incontinece, brain ischemia, multiple sclerosis, Alzheimer's disease, senile dementia of the Alzheimer's type, mild cognitive impairment, age-related cognitive decline, emesis, corticobasal degeneration, dementia pugilistica, Down's syndrome, myotonic dystrophy, Niemann-Pick disease, Pick's disease, prion disease with tangles, progessive supranuclear palsy, lower lateral sclerosis, and subacute sclerosing panencephalistis.

27. A method for treating a disease or condition the treatment of which can be effected or facilitated by altering dopamine mediated neurotransmission in a mammal comprising administering to the mammal a compound according to claim 1 in an amount effective in treating said disease or condition.

28. A method according to claim 27 wherein the disease or condition is selected from Parkinson's disease, schizophrenia, schizophreniform disorder, schizoaffective disorder, delusional disorder, substance-induced psychotic disorder, personality disorder of the paranoid type, personality disorder of the schizoid type, drug addiction, drug withdrawal, obsessive compulsive disorder, Tourette's syndrome, depression, a mood episode, post-stroke depression, major depressive disorder, dysthymic disorder, minor depressive disorder, premenstrual dysphoric disorder, post-psychotic depressive disorder of schizophrenia, a major depressive disorder superimposed on a psychotic disorder such as delusional disorder or schizophrenia, a bipolar disorder, anxiety; attention deficit and hyperactivity disorder; and attention deficit disorder.

29. A method for treating in a mammal a disease or condition selected from male fertility and sperm motility; diabetes mellitus; impaired glucose tolerance; metabolic syndrome or syndrome X; polycystic ovary syndrome; adipogenesis and obesity; myogenesis and frailty, for example age-related decline in physical performance; acute sarcopenia, for example muscle atrophy and/or cachexia associated with burns, bed rest, limb immobilization, or major thoracic, abdominal, and/or orthopedic surgery; sepsis; hair loss, hair thinning, and balding; and immunodeficiency; which method comprises administering to said mammal an amount of a compound according to claim 1 effective in treating said disease or condition.

30. A method for inhibiting GSK-3 in a mammal, which method comprises administering to said mammal an amount of a compound according to claim 1 effective in inhibiting GSK-3.

31. A pharmaceutical composition for treating depression or anxiety in a mammal comprising a compound according to claim 1 and
a) an NK-1 receptor antagonist;
b) a 5HT_{1D} receptor antagonist; or
c) an SSRI;
wherein the compound according to claim 1 and a), b), or c) are together in an amount effective in treating depression or anxiety, and a pharmaceutically acceptable carrier.

32. A method for treating depression or anxiety in a mammal which method comprises administering to said mammal a compound according to claim 1 and
a) an NK-1 receptor antagonist;
b) a 5HT_{1D} receptor antagonist; or
c) an SSRI;
wherein the combined amounts of the compound of claim 1 and a), b), or c) are effective in treating depression or anxiety.

33. A pharmaceutical composition for treating schizophrenia in a mammal comprising a compound according to claim 1 and as antipsychotic selected from ziprasidone, olanzapine, risperidone, L-745870, sonepiprazole, RP 62203, NGD 941, balaperidone, flesinoxan, and gepirone, together in an amount effective in treating schizophrenia, and a pharmaceutically acceptable carrier.

34. A method of treating schizophrenia in a mammal which method comprises administering to said mammal a compound according to claim 1 and an antipsychotic selected from ziprasidone, olanzapine, risperidone, L-745870, sonepiprazole, RP 62203, NGD 941, balaperidone, flesinoxan, and gepirone, wherein the combined amounts of the compound of claim 1 and the antipsychotic are effective in treating schizophrenia.

35. A pharmaceutical composition for treating a disorder selected from Alzheimer's disease, mild cognitive impairment, and age-related cognitive decline in a mammal comprising a compound according to claim 1 and an acetylcholinesterase inhibitor together in an amount effective in treating said disorder, and a pharmaceutically acceptable carrier.

36. A method for treating in a mammal a disorder selected from Alzheimer's disease, mild cognitive impairment, and age-related cognitive decline, which method comprises administering to said mammal a compound according to claim 1 and an acetylcholinesterase inhibitor, wherein the combined amounts of the compound of claim 1 and the acetylcholinesterase inhibitor are effective in treating said disorder.

37. A pharmaceutical composition for treating a disease or condition selected from stroke, spinal cord trauma, traumatic brain injury, multiinfarct dementia, epilepsy, pain, Alzheimer's disease, and senile dementia in a mammal comprising a compound of claim 1 and
a) TPA; or
b) NIF;
wherein the compound of claim 1 and TPA or NIF are together in an amount effective in treating said disorder, and a pharmaceutically acceptable carrier.

38. A method for treating in a mammal a disease or condition selected from stroke, spinal cord trauma, traumatic brain injury, multiinfarct dementia, epilepsy, pain, Alzheimer's disease, and senile dementia, which method comprises administering to said mammal a compound of claim 1 and
a) TPA; or
b) NIF;
wherein the combined amounts of the compound of claim 1 and TPA or NIF are effective in treating said disease or condition.

39. A pharmaceutical composition for treating a disease or condition selected from Huntington's disease, stroke, spinal cord trauma, traumatic brain injury, multiinfarct dementia, epilepsy, amyotrophic lateral sclerosis, pain, viral induced dementia for example AIDS induced dementia, migraine, hypoglycemia, urinary incontinece, brain ischemia, multiple sclerosis, Alzheimer's disease, senile dementia of the Alzheimer's type, mild cognitive impairment, age-related cognitive decline, emesis, corticobasal degeneration, dementia pugilistica, Down's syndrome, myotonic dystrophy, Niemann-Pick disease, Pick's disease, prion disease with tangles, progessive supranuclear palsy, lower lateral sclerosis, and subacute sclerosing panencephalistis in a mammal comprising a compound of claim 1 and an NMDA receptor antagonist together in an amount effective in treating said disorder, and a pharmaceutically acceptable carrier.

40. A method for treating in a mammal a disease or condition selected from Huntington's disease, stroke, spinal cord trauma, traumatic brain injury, multiinfarct dementia, epilepsy, amyotrophic lateral sclerosis, pain, viral induced dementia for example AIDS induced dementia, migraine, hypoglycemia, urinary incontinece, brain ischemia, multiple sclerosis, Alzheimer's disease, senile dementia of the Alzheimer's type, mild cognitive impairment, age-related cognitive decline, emesis, corticobasal degeneration, dementia pugilistica, Down's syndrome, myotonic dystrophy, Niemann-Pick disease, Pick's disease, prion disease with tangles, progessive supranuclear palsy, lower lateral sclerosis, and subacute sclerosing panencephalistis, which method comprises administering to said mammal a compound of claim 1 and an NMDA receptor antagonist, wherein the combined amounts of the compound of claim 1 and the NMDA receptor antagonist are effective in treating said disease or condition.

41. A pharmaceutical composition for treating a disease or condition selected from stroke, spinal cord trauma, traumatic brain injury, multiinfarct dementia, epilepsy, pain, Alzheimer's disease, and senile dementia in a mammal comprising a compound of claim 1 and a potassium channel modulator together in an amount effective in treating said disorder, and a pharmaceutically acceptable carrier.

42. A method for treating in a mammal a disease or condition selected from stroke, spinal cord trauma, traumatic brain injury, multiinfarct dementia, epilepsy, pain, Alzheimer's disease, and senile dementia, which method comprises administering to said mammal a compound of claim 1 and a potassium channel modulator, wherein the combined amounts of the compound of claim 1 and the potassium channel modulator are effective in treating said disease or condition.

43. A compound of the formula 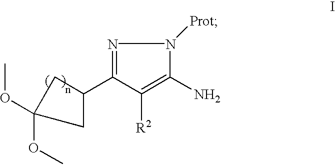
wherein Prot is a protecting group;
R² is H, F, —CH₃, —CN, or —C(═O)OR⁷;
wherein R⁷ is selected from H, straight chain or branched (C₁-C₈)alkyl, straight chain or branched (C₂-C₈)alkenyl, straight chain or branched (C₂-C₈ alkynyl), (C₃-C₈)cycloalkyl, ( C₄-C₈)cycloalkenyl, (3-8 membered) heterocycloalkyl, (C₅-C₁₁)bicycloalkyl, (C₇-C₁₁)bicycloalkenyl, (5-11 membered) heterobicycloalkyl, (C₆-C₁₄)aryl, and (5-14 membered) heteroaryl, wherein R⁷, is optionally substituted with from one to six substituents independently selected from F, Cl, Br, I, NO₂, —CN, —CF₃, —NR¹⁰R¹¹, —NR¹⁰ C(═O)R¹¹, —NR¹⁰C(═O)OR¹¹, —NR¹⁰C(═O)NR¹¹R¹², —NR¹⁰ S(═O)₂R¹¹, —NR¹⁰S(═O)₂NR¹¹R¹², —OR¹⁰, —OC(═O)R¹⁰, —OC(═O)OR¹⁰, —OC(═O)NR¹⁰R¹¹, —OC(═O)SR¹⁰, —SR¹⁰, —S(═O)R¹⁰, —S(═O)₂R¹⁰, —S(═O)₂NR¹⁰R¹¹, —C(═O)R¹⁰, —C(═O)OR¹⁰, —C(═O)NR¹⁰R¹¹, and R^{10 ;}
each R¹⁰, R¹¹, and R¹² is independently selected from H, straight chain or branched (C₁-C₈)alkyl, straight chain or branched (C₂-C₈)alkenyl, straight chain or branched (C₂-C₈ alkynyl), (C₃-C₈)cycloalkyl, (C₄-C₈)cycloalkenyl, (3-8 membered) heterocycloalkyl, (C₅-C₁₁)bicycloalkyl, (C₇-C₁₁)bicycloalkenyl, (5-11 membered) heterobicycloalkyl, (C₆-C₁₄)aryl, and (5-14 membered) heteroaryl, wherein R¹⁰, R¹¹, and R¹² are each independently optionally substituted with from one to six substituents independently selected from F, Cl, Br, I, NO₂, —CN, —CF₃, —NR¹³R¹⁴, —NR¹³C(═O)R¹⁴, —NR¹³C(═O)OR¹⁴, —NR¹³C(═O)NR¹⁴R¹⁵, —NR¹³S(═O)₂R¹⁴, —NR¹³S(═O)₂NR¹⁴R¹⁵, —OR¹³, —OC(═O)R¹³, —OC(═O)OR¹³, —OC(═O)NR¹³R¹⁴, —OC(═O)SR¹³, —SR¹³, —S(═O)R¹³, —S(═O)₂R¹³, —S(═O)₂NR¹³R¹⁴, —C(═O)R¹³, —C(═O)OR¹³, —C(═O)NR¹³R¹⁴, and R¹³;
each R¹³, R¹⁴, and R¹⁵ is independently selected from H, straight chain or branched (C₁-C₈)alkyl, straight chain or branched (C₂-C₈)alkenyl, straight chain or branched (C₂-C₈ alkynyl), (C₃-C₈)cycloalkyl, (C₄-C₈)cycloalkenyl, (3-8 membered) heterocycloalkyl, (C₅-C₁₁)bicycloalkyl, (C₇-C₁₁)bicycloalkenyl, (5-11 membered) heterobicycloalkyl, (C₆-C₁₄)aryl, and (5-14 membered) heteroaryl, wherein R¹³, R¹⁴, and R¹⁵ are each independently optionally substituted with from one to six substituents independently selected from F, Cl, Br, I, NO₂, —CN, —CF₃, —NR¹⁶R¹⁷, —NR¹⁶(═O)R¹⁷, —NR¹⁶C(═O)OR¹⁷, —NR¹⁶C(═O)NR¹⁷R¹⁸, —NR¹⁶S(═O)₂R¹⁷, —NR¹⁶S(═O)₂NR¹⁷R¹⁸ —OR¹⁶, —OC(═O)R¹⁶, —OC(═O)OR¹⁶, —OC(═O)NR¹⁶R¹⁷, —OC(═O)SR¹⁶, —SR¹⁶, —S(═O)R¹⁶, —S(═O)₂R¹⁶, —S(═O)₂NR¹⁶R¹⁷, —C(═O)R¹⁶, —C(═O)OR¹⁶, —C(═O)NR¹⁶R¹⁷, and R¹⁶. each R¹⁶, R¹⁷, and R¹⁸ is independently selected from H, straight chain or branched (C₁-C₈)alkyl, straight chain or branched (C₂-C₈)alkenyl, straight chain or branched (C₂-C₈ alkynyl), (C₃-C₈)cycloalkyl, (C₄-C₈)cycloalkenyl, (3-8 membered) heterocycloalkyl, (C₅-C₁₁)bicycloalkyl, (C₇-C₁₁)bicycloalkenyl, (5-11 membered) heterobicycloalkyl, (C₆-C₁₄)aryl, and (5-14 membered) heteroaryl;
and n is an integer selected from 1, 2, 3, and 4.
